(19) United States
(12) Patent Application Publication
(10) Pub. No.: US 2009/0202475 A1
(43) Pub. Date: Aug. 13, 2009

(54) Compositions and methods for treatment of microbial disorders

(75) Inventors: Alexander R. Abbas, San Carlos, CA (US); Nico P. Ghilardi, Millbrae, CA (US); Zora Modrusan, Fremont, CA (US); Dimitry M. Danilenko, Millbrae, CA (US); Frederic J. De Sauvage, Foster City, CA (US); Wenjun Ouyang, Foster City, CA (US); Patricia A. Valdez, Kensington, MD (US); Yan Zheng, Fremont, CA (US)

Correspondence Address: Goodwin Procter LLP;Attn: Patent Administrator, 135 Commonwealth Drive, Menlo Park, CA, 94025-1105, US

(73) Assignee: Genentech, Inc., South San Francisco, CA (US)

(21) Appl. No.: 12/291,380

(22) Filed: Nov. 7, 2008

Related U.S. Application Data
(60) Provisional application No. 60/986,170, filed on Nov. 7, 2007.
(60) Provisional application No. 61/013,620, filed on Dec. 13, 2007.
(60) Provisional application No. 61/015,620, filed on Dec. 20, 2007.

(51) Int. Cl. A61K 38/20; A61K 38/16; C12N 5/00; A61K 38/02; A61K 31/7052; A61K 39/395; A61P 31/00; C07K 14/54; C07K 14/00

(52) U.S. Cl. 424/85.2; 514/12; 435/375; 514/2; 424/134.1; 424/158.1; 424/145.1; 424/133.1; 530/351; 530/350

(57) ABSTRACT

The present invention relates to compositions and methods for treatment of microbial disorder by modulation of the host immune response. More particularly, the present invention relates to compositions that mediate an anti-microbial immune response, and methods of treating a microbial infection using such compositions.

### CROSS-REFERENCE TO RELATED APPLICATIONS

This application claims priority under Section §119(e) and the benefit of U.S. Provisional Application Ser. No. 60/986,170 filed Nov. 7, 2007, U.S. Provisional Application Ser. No. 61/013,620 filed Dec. 13, 2007, U.S. Provisional Application Ser. No. 61/015,620 filed Dec. 20, 2007, the entire disclosures of which are incorporated herein by reference in their entirety.

### FIELD OF THE INVENTION

The present invention relates generally to the treatment of microbial disorders by modulation of the host immune response.

### BACKGROUND

Infection by microbial pathogens represents a major cause of death worldwide and continues to pose a serious threat to global health (WHO, The World Health Report (2004)). For example, Attaching and effacing (A/E) bacterial pathogens, such as enterohemorrhagic Escherichia coli (EHEC) and enteropathogenic E. coli (EPEC) are among the bacteria that cause diarrhea, morbidity and mortality, especially among infants and children in the developing world (2). E. coli O157:H7, one of the EHEC strains, caused many people to be hospitalized and 3 mortalities last year in the United States (MMWR Morb Mortal Wkly Rep 55, 1045 (Sep. 29, 2006)). It is also believed that more than 90% of all cases of post-diarrhea hemolytic uremic syndrome (HUS) in industrialized countries were caused by E. coli O157:H7 infection (R. L. Siegler, Pediatr Clin North Am 42, 1505 (December, 1995)). Other EPEC strains such as E. coli 055:H7 also cause intestinal illness among infants world wide (T. S. Whittam et al., Infect Immun 61, 1619 (May, 1993)). Much of our knowledge on how hosts control the infection of A/E pathogens comes from the study of infection by Citrobacter rodentium, a natural pathogen in mice (L. Eckmann, Ann NY Acad Sci 1072, 28 (Aug. 1, 2006)). Similar to the pathogenesis of EHEC or EHPC in human, intimately attaching of C. rodentium to murine colonic epithelial cells results in effacement of brush border microvilli, termed as attaching and effacing (A/E) lesion, and colonic hyperplasia (D. B. Schauer, S. Falkow, Infect Immun 61, 2486 (June, 1993)).

Both intestinal epithelial and immune cells play critical roles in host defense against A/E-pathogens. The tight junctions of intestinal epithelial cells present the first barrier to prevent microbes leaving the intestinal lumen (T. T. MacDonald, G. Monteleone, Science 307, 1920 (Mar. 25, 2005)). Additionally, epithelial cells secrete anti-microbial peptides to control pathogens in the gastrointestinal (GI) tract (A. Takahashi et al., FEBS Lett 508, 484 (Nov. 23, 2001)). Studies with immune deficient mouse strains during C. rodentium infection established that CD4⁺ T cells, B cells, and anti-C. rodentium specific antibody responses are all essential components of the adaptive immunity to contain and eradicate infection (L. Bry, M. B. Brenner, J Immunol 172, 433 (Jan. 1, 2004)). Many cytokines produced by lymphocytes during infection can enhance the innate immune responses of epithelial cells. The specific functions of these cytokines, however, remain unclear during A/E pathogen infection.

IL-22, an IL-10 family cytokine, is produced by lymphocytes, particularly Th17 cells (Y. Zheng et al., Nature 445, 648 (Feb. 8, 2007)). Th17 cells belong to a recently discovered CD4⁺ T helper subset that also produces IL-17. IL-17 has important functions in the control of extracellular bacterial infections (K. I. Happel et al., J. Exp. Med. 202, 761 (Sep. 19, 2005)). The role of IL-22, however, in host defense is still largely unknown. Tumor Necrosis Factor (TNF)-related proteins are recognized in the art as a large family of proteins having a variety of activities ranging from host defense to immune regulation to apoptosis. TNF was first identified as a serum-derived factor that was cytotoxic for several transformed cell lines in vitro and caused necrosis of certain tumors in vivo. A similar factor in the superfamily was identified and referred to as lymphotoxin (“LT”). Due to observed similarities between TNF and LT in the early 1980's, it was proposed that TNF and LT be referred to as TNF-α and TNF-β, respectively. Scientific literature thus makes reference to both nomenclatures. As used in the present application, the term “INF” refers to TNF-α. Later research revealed two forms of lymphotoxin, referred to as LTα and LTβ. US 2005-0129614 describes another polypeptide member of the TNF ligand super-family based on structural and biological similarities, designated TL-5. Members of the TNF family of proteins exist in membrane-bound forms that act locally through cell-cell contact, or as secreted proteins. A family of TNF-related receptors react with these proteins and trigger a variety of signalling pathways including cell death or apoptosis, cell proliferation, tissue differentiation, and proinflammatory responses. TNF-α by itself has been implicated in inflammatory diseases, autoimmune diseases, viral, bacterial, and parasitic infections, malignancies, and/or neurodegenerative diseases and is a useful target for specific biological therapy in diseases such as RA and Crohn's disease.

### SUMMARY OF THE INVENTION

The present invention provides compositions and methods for treatment of microbial disorders by modulation of the host immune response. For example, an anti-microbial immune response in a host can be enhanced or inhibited by increasing or decreasing an activity of one or more anti-microbial polypeptides (AMPs) that mediate the anti-microbial immune response.

More particularly, the present invention provides AMPs, modulators thereof, and methods of using such compositions for treatment of microbial disorders. Such microbial disorders include, but are not limited to, infectious diseases, for example, EHEC- and EPEC-caused diarrhea, Inflammatory Bowel Disease (IBD) and, more particularly, Ulcerative Colitis (UC) and Crohn's Disease (CD).

AMPs of the present invention are polypeptides that mediate an anti-microbial immune response, and include, but are not limited to, LT, IL-6, IL-18, IL-22, IL-23 (including e.g., IL-23 p19 or IL-23 p40), and Reg or Reg-related proteins encoded by the genes of the Reg super family. The Reg super family includes Reg and Reg-related genes from human, rat, and mouse and are grouped into four subclasses, types I, II, III, and IV. For example, type I includes human REG Iα, human REG Iβ, rat RegI, and mouse RegI; type II includes mouse RegII; type III includes human REG III, human HIP/PAP (gene expressed in hepatocellular carcinoma-intestine-pancreas/gene encoding pancreatitis-associated protein), rat PAP/Peptide23, rat RegIII/PAPII, rat PAP III, mouse RegIIIα, RegIIIβ, RegIIIγ, mouse RegIIIδ, and hamster INGAP (islet neogenesis-associated protein). Type IV contains human REG IV. In one aspect, the REG protein is encoded by a member of the human REG gene family which includes, but is not limited to, REG Iα, REG Iβ, HIP/PAP, REG III, REG IV, and Reg-related sequence (RS).

In some aspects, the amino acid sequence of an AMP of the present invention comprises an amino acid sequence selected from the following group: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, and SEQ ID NO: 56.

In other aspects, the nucleic acid sequence encoding an AMP of the present invention comprises a nucleic acid sequence selected from the following group: SEQ ID NO: 1, SEQ ID NO: 3, SEQ ID NO: 5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, and SEQ ID NO: 55.

An activity of an AMP of the present invention can be increased or decreased and/or differentially regulated relative to the activity of another AMP or the same AMP. Examples of an activity of an AMP of the present invention, includes, but is not limited to, AMP expression, binding to a binding partner, signal transduction, anti-microbial activity, or other biological or immunological activity thereof.

In one aspect, an increase in the activity of one or more AMPs of the present invention results in an enhanced anti-microbial immune response in a subject.

In one aspect, AMPs of the present invention include, but are not limited to, polypeptides that directly or indirectly interact with IL-22, e.g., polypeptides that are upstream or downstream of an IL-22 signal transduction pathway that mediates host resistance to infection by a microbial pathogen (e.g., a bacteria or virus). Examples of such AMPs include, but are not limited to, LT, L-6, L-18, and IL-23 (including e.g., IL-23 p19 or IL-23 p40).

Modulators of the present invention include, but are not limited to, polypeptides and nucleic acid molecules (e.g., a DNA molecule or RNA molecule) that directly or indirectly modulate an activity of an AMP. Examples of such modulation include, but are not limited to, an increase, decrease, induction or activation, inhibition, or regulation (e.g., up or down regulation) of an activity of an AMP of the present invention.

In one aspect, the modulator indirectly modulates IL-22 activity by decreasing or inhibiting IL-22 Binding Protein (BP) activity and thereby, increasing IL-22 activity. In a particular aspect, the modulator decreases or inhibits binding of IL-22 BP to IL-22 and thereby, increases IL-22 activity.

In some aspects, the modulator is a polypeptide e.g., a polypeptide that binds to or otherwise interacts with an AMP to increase, induce, or regulate an activity of an AMP. In one aspect, the modulator is a fusion polypeptide that modulates an activity of an AMP.

In one aspect, the modulator is an antibody that binds to an AMP. In a particular aspect, the antibody is a monoclonal antibody. In another aspect, the antibody is an antibody fragment selected from a Fab, Fab′-SH, Fv, scFv, or (Fab′)₂ fragment. In another aspect, the antibody is a fusion polypeptide (e.g., an Fc fusion polypeptide). In another aspect, the antibody is a chimeric antibody. In a particular aspect, the antibody is humanized. In another aspect, the antibody is a human antibody. In another aspect, the antibody binds to the same epitope as an antibody selected from a human, non-human primate, or other mammal (e.g., pig, sheep, rabbit, marmot, rat, or mouse). In a particular aspect, the antibody is an AMP agonist.

In another particular aspect, the modulator is a recombinant AMP or nucleic acid molecule encoding an AMP (e.g., a DNA or RNA molecule).

The present invention further provides methods of treating a microbial disorder by modulating an anti-microbial immune response. In one aspect, the present invention provides a method of treating a microbial disorder, in a subject, comprising administering to the subject an effective amount of pharmaceutical composition comprising an AMP or modulator of the AMP, wherein the AMP is selected from a group consisting of: LT, IL-6, IL-18, IL-22, IL-23, REG Iα, REG Iβ, HIP/PAP, REG III, REG IV and Reg-related sequence (RS). In one aspect the disorder is an infectious disease, for example, EHEC- or EPEC-caused diarrhea, Inflammatory Bowel Disease (IBD) or, more particularly, Ulcerative Colitis (UC) or Crohn's Disease (CD).

In particular aspects, the present invention provides methods of modulating an anti-microbial immune response by stimulating or inhibiting an AMP-mediated signaling pathway and/or Th_{IL-17} cell function. Such methods are useful for treatment of microbial disorders. For example, in one aspect, the present invention provides a method of enhancing an anti-microbial immune response by stimulating an AMP-mediated signaling pathway, e.g., and IL-22 and/or IL-23 mediated signaling pathway. In another aspect, the present invention provides methods of modulating an anti-microbial immune response by stimulating or inhibiting a cytokine-mediated signaling pathway. For example, in one aspect, the present invention provides methods of enhancing an anti-microbial immune response by stimulating a cytokine-mediated signaling pathway, e.g., an IL-22 and/or IL-23 signaling pathway. Moreover, the present invention provides methods of modulating an anti-microbial immune response by stimulating or inhibiting a Th_{IL-17} cell function.

In one aspect, the present invention provides a method of stimulating an AMP-mediated signaling pathway in a biological system, the method comprising providing an AMP agonist to the biological system. Examples of such a biological system include, but are not limited to, mammalian cells in an in vitro cell culture system or in an organism in vivo. In another aspect, the present invention provides a method of inhibiting an AMP-mediated signaling pathway in a biological system, the method comprising providing an AMP antagonist to the biological system.

In a particular aspect, the present invention provides a method of enhancing an anti-microbial immune response in a biological system by stimulating an IL-23 and/or IL-22 mediated signaling pathway in a biological system, the method comprising providing an IL-22 or IL-22 agonist to the biological system. In one aspect, an IL-22 agonist is IL-22. In another aspect, the IL-22 agonist is an antibody that binds to IL-22.

In another aspect, a method of inhibiting an IL-23-mediated signaling pathway in a biological system is provided, the method comprising providing an IL-22 antagonist to the biological system. In one aspect, the antagonist of IL-22 is an antibody, e.g., a neutralizing anti-IL-22 antibody and/or a neutralizing anti-IL-22R antibody.

In another aspect, the present invention provides a method of stimulating a Th_{IL-17} cell function, the method comprising exposing a Th_{IL-17} cell to an agonist of an AMP that mediates the IL-23 mediated signaling pathway (e.g., IL-23, IL-6, or IL-22). Such methods are useful for treating a microbial disorder.

In one aspect, an IL-22 agonist is IL-22. In another aspect, the IL-22 agonist is an antibody that binds to IL-22.

In another aspect, a method of inhibiting a Th_{IL-17} cell function is provided, the method comprising exposing a Th_{IL-17} cell to an antagonist of an AMP that mediates the IL-23 mediated signaling pathway (e.g., IL-23, IL-6, or IL-22). In one aspect the antagonist is an anti-IL-22 antibody, e.g., a neutralizing anti-IL-22 antibody.

Exemplary Th_{IL-17} cell functions include, but are not limited to, stimulation of cell-mediated immunity (delayed-type hypersensitivity); recruitment of innate immune cells, such as myeloid cells (e.g., monocytes and neutrophils) to sites of inflammation; and stimulation of inflammatory cell infiltration into tissues. In one aspect, a Th_{IL-17} cell function is mediated by IL-23 and/or IL-22.

In a further aspect, the present invention provides a method of treating an infection by a microbial pathogen (e.g., a bacteria or virus), in a subject, comprising administering to the subject an effective amount of pharmaceutical composition comprising an AMP or modulator of the AMP, wherein the AMP is selected from a group consisting of: LT, IL-6, IL-18, IL-22, IL-23, REG Iα, REG Iβ, HIP/PAP, REG III, REG IV and Reg-related sequence (RS).

In another aspect, the present invention provides a method of treating a microbial disorder, in a subject, comprising contacting cells of the subject with a nucleic acid molecule (e.g., a DNA or RNA molecule) encoding an AMP or modulator of the AMP, wherein the AMP is selected from a group consisting of: LT, IL-6, IL-18, IL-22, L-23, REG Iα, REG Iβ, HIP/PAP, REG III, REG IV and Reg-related sequence (RS). In one aspect the disorder is an infectious disease, for example, EHEC- or EPEC-caused diarrhea, Inflammatory Bowel Disease (IBD) or, more particularly, Ulcerative Colitis (UC) or Crohn's Disease (CD).

In another aspect, the present invention provides a method of modulating the activity of an AMP in cells of a subject infected with a microbial pathogen (e.g., a bacteria or virus), comprising contacting the cells with a nucleic acid molecule (e.g., a DNA or RNA molecule) encoding an AMP or modulator of the AMP, wherein the AMP is selected from a group consisting of: LT, IL-6, IL-18, IL-22, IL-23, REG Iα, REG Iβ, HIP/PAP, REG III (e.g., REG IIIβ or REGIIIγ), REG IV, and Reg-related sequence (RS).

Examples of a microbial pathogen include, but are not limited to, a bacteria or virus. In one aspect, the microbial pathogen is a bacteria e.g., a gram-negative or gram-positive bacteria. In a particular aspect, the bacteria is a gram-negative bacteria. In another aspect, the bacteria is an attaching or effacing (A/E) bacteria and, more particularly, an enterohemorrhagic Escherichia coli (EHEC) or enteropathogenic 1. Coli (EPEC). In one aspect, the bacteria is enteropathogenic E. coli (EHEC) is E. coli 0157:H7 or E. coli 055:H7.

In another aspect, the present invention provides polynucleotides encoding an AMP of the present invention, or modulator thereof. In another aspect, the invention provides a vector comprising the polynucleotide. In another aspect, the invention provides a host cell comprising the vector. In one aspect, the host cell is a eukaryotic cell. In another aspect, the host cell is a CHO cell, yeast cell, or bacterial cell (e.g., E. coli).

In one aspect, the present invention provides a method of making an antibody that binds to an AMP of the present invention, wherein the method comprises culturing the host cell under conditions suitable for expression of the polynucleotide encoding the antibody, and isolating the antibody. In a particular aspect, the invention provides a method of making an antibody that is an agonist of an AMP of the present invention.

In one aspect, the present invention provides a method of detecting the presence of an AMP in a biological sample, comprising contacting the biological sample with an antibody to the AMP, under conditions permissive for binding of the antibody to the AMP, and detecting whether a complex is formed between the antibody and AMP.

In another aspect, the present invention provides a kit comprising one or more AMPs of the present invention and/or modulators thereof. In another aspect, the present invention provides a kit comprising one or more one or more pharmaceutical compositions each comprising an AMP of the present invention or modulator thereof.

### BRIEF DESCRIPTION OF THE DRAWINGS

FIG. 1 depicts data demonstrating host defense against C. rodentium infection. FIG. 1(A) depicts the results of a real-time RT-PCR analysis on receptor subunits for IL-22 in uninfected wildtype mouse G1 track; FIG. 1(B-F) depicts a real-time RT-PCR analysis on various cytokine expressions in wildtype mouse colons upon C. rodentium infection; FIG. 1(G) depicts survival of C57Bl/6 (n=5), IL-23p40^{-/-} (n=5), and IL-6^{-/-} (n=5) mice after C. rodentium infection; and FIG. 1(H) depicts a time course real-time RT-PCR analysis on IL-22 and IL-17 expressions in C57Bl/6, IL-23p40^{-/-}, and IL-6^{-/-} mouse colons upon C. rodentium infection. For C. rodentium infection, the mice were orally inoculated with 2×10⁹ CFU of bacteria. All of the above data are representative of two independent experiments.

FIG. 2 depicts data demonstrating that IL-22 deficiency renders mice susceptible to C. rodentium infection. 6-7 weeks old IL-22^{-/-} (FIG. 2(A-C)), IL-17RC^{-/-} (D), IL-20Rβ^{-/-} mice (FIG. 2(F)) or wildtype mice (FIG. 2(A-C, E)) were orally inoculated with 2×10⁹ CFU of C. rodentium and weighed at indicated time points. Histologic analysis of colons from IL-22^{-/-} and wildtype mice 8 days post inoculation using hematoxylin-and-eosin (H&E) staining (FIGS. 2(B and C)). Arrows indicate submucosal inflammation (FIG. 2(B)), and bacterial invasion into mucosal glands (FIG. 2(C)). Representative data are shown (bars=100 μm for FIG. 2(B) and bars=25 μm for FIG. 2(C)). Wildtype C57Bl/6 mice received 150 μg of anti-IL-22 mAb or isotype control IgG1 mAb intraperitoneally, every other day, starting on day 0 or day 8 post inoculation (FIG. 2(E)). *p<0.05, **p<0.01, ***p<0.001. All data are representative of two independent experiments.

FIG. 3 depicts data demonstrating the effect of IL-22 deficiency in mice during C. rodentium infection. C57Bl/6 mice (FIGS. 3(A, B, and F)), IL-22^{-/-} and wildtype mice (FIGS. 3(C-E, and G)) were orally inoculated with 2×10⁹ CFU of C. rodentium. Mice also received 150 μg of anti-IL-22 mAb or isotype control IgG1 mAb intraperitoneally every other day starting from the same day as C. rodentium inoculation (FIGS. 3(A and B)). On day 10, colons were photographed and individual colon length was measured (FIG. 3(A)). Histologic analysis of colons was performed using hematoxylin-and-eosin (H&E) staining (FIG. 3(B)). Histologic analysis of colons and livers (day 8) from infected IL-22^{-/-} and wildtype mice was performed using H&E staining (FIGS. 3(C and E)). Arrows in FIG. 3(C) indicate colonic transmural inflammation and ulceration. FIG. 3(E) depicts a hepatic septic microabscess in the IL-22^{-/-} mouse. Representative data are shown in FIG. 3(C), where the bars=500 μm for the upper panels and bars=100 μm for the lower panels. In FIG. 3(E), the bars=25 μm. FIG. 3(D) depicts the log₁₀ CFU of C. rodentium in colon, liver, spleen, and mesenteric lymph node. FIG. 3(F-G) depicts the serum anti-C. rodentium IgG levels by ELISA. * p<0.05. All of the above data are representative of two independent experiments.

FIG. 4 depicts data demonstrating that IL-22 induces anti-microbial RegIII family protein expression upon C. rodentium infection. In vitro culture of C57Bl/6 mouse colons were treated with 10 μg of IL-22 for 24 hours, RNA were isolated and used for microarray analysis (FIG. 4(A)) and real-time RT-PCR analysis (FIG. 4(B)). In FIG. 4(C), IL-22^{-/-} mice and wildtype littermates were orally inoculated with 2×10⁹ CFU of C. rodentium, and real-time RT-PCR was performed on RNA isolated from individual mouse colon collected on indicated time points. All data are representative of two independent experiments.

FIG. 5 depicts data demonstrating the targeted disruption of the murine IL-17RC gene. FIG. 5(A) depicts the strategy for generation of IL-17RC knockout mice. Exons 1-5 (open boxes) encompassing the IL-17RC coding sequence was replaced with a neomycin resistance cassette. FIG. 5(B) depicts the genotyping of offspring from wildtype (WT) and knockout (KO) mice using the indicated primer sets (P1, P2 and P3). Tail tip fibroblasts from WT and KO mice were generated and stimulated with various concentrations of IL-17A and IL-17F in vitro for 24 hours, and culture supernatant were collected for IL-6 ELISA (FIG. 5(C)).

FIG. 6 depicts data of a real-time RT-PCR analysis on IL-19, IL-20 and IL-24 expression in wildtype mouse colons upon C. rodentium infection, over time. C57Bl/6 mice were orally inoculated with 2×10⁹ CFU of C. rodentium. Colons were collected at indicated time points and isolated RNAs were used for real-time RT-PCR analysis.

FIG. 7 depicts data demonstrating IL-20Rα and IL-20Rβ expression in the GI tract. Real-time RT-PCR analysis on receptor subunits for IL-19, IL-20 and IL-24 in uninfected wildtype mouse GI tract.

FIG. 8 depicts data demonstrating targeted disruption of the murine IL-20Rβ gene. FIG. 8(A) depicts the strategy for generation of IL-20Rβ knockout mice. Exon 1 (open boxes) was replaced with a neomycin resistance cassette. FIG. 8(B) depicts the phenotyping of offspring from wildtype (WT), heterozygous (HET) and knockout (KO) mice using the indicated primer sets (p1, p2 and p3). FIG. 8(C) WT and KO mouse ears were injected intradermally with 500 ng recombinant IL-20 in 20 μl PBS or with 20 μl PBS alone. 24 hours later, mouse ears were collected for RNA isolation. Isolated RNAs were used for real-time RT-PCR analysis for genes known to be upregulated upon IL-20 signaling.

FIG. 9 depicts data of a histologic analysis of mouse colons from anti-IL-22 mAb treated wildtype mice inoculated with C. rodenlium. C57Bl/6 mice were orally inoculated with 2×10⁹ CFU of C. rodentium. Mice also received 150 μg of anti-IL-22 mAb or isotype control IgG1 mAb intraperitoneally every other day starting from the same day as C. rodentium inoculation. On day 10, routine histologic analysis of colons was performed using hematoxylin-and-eosin (H&E) staining. Arrows indicate mucosal ulceration with transmural inflammation. Representative images are shown, bars=500 μm for the upper panels and bars=250 μm for the lower panels.

FIG. 10 depicts data demonstrating serum Ig levels in IL-22^{-/-} mice and wildtype littermates during C. rodentium infection. IL-22^{-/-} and wild type littermates mice were orally inoculated with 2×10⁹ CFU of C. rodentium. On indicated time points, mouse blood were collected. Levels of total serum IgM and IgG (FIG. 10(A)) and serum anti-C. rodentium IgG2a, IgG2b, IgG2c and IgG3 (FIG. 10(B)) were determined by ELISA. All data are representative of two independent experiments.

FIG. 11 depicts data demonstrating an ex vivo colon culture ELISA of IL-22 (FIG. 11(A)) and IL-17 (FIG. 11(B)) expression in C57Bl/6, IL-23p19^{-/-}, and IL-6^{-/-} mouse colons after C. rodentium infection. For C. rodentium infection, mice were orally inoculated with 2×10⁹ CFU of bacteria. All data are representative of at least two independent experiments.

FIG. 12 depicts a FACS analysis of IL-22R expression on isolated mouse IEL, LPMCs and colonic epithelial cells (FIG. 12 (A)), and a FACS analysis of IL-22R expression on primary human colonic epithelial cells (FIG. 12 (B)). All data are representative of at least two independent experiments.

FIG. 13 depicts data demonstrating that IL-22, produced by dendritic cells (DCs), is critical for innate immune responses against C. rodentium infection. In FIG. 13(A), Rag2^{-/-} and wildtype Balb/c mice were orally inoculated with 2×10⁹ CFU of C. rodentium. In FIGS. 13(B and C), the mice also received 150 μg of isotype control IgG1 mAb or anti-IL-22 mAb intraperitoneally every other day starting at the same day as bacteria inoculation and were weighed at the indicated time points. FIG. 13(B) depicts a time course real-time RT-PCR analysis, and FIG. (13(C) depicts an ex vivo colon culture ELISA of IL-22 and IL-17 expression in colons of wildtype Balb/c and Rag2^{-/-} mice following C. rodentium infection. FIG. 13(D) depicts the immunohistochemical staining for IL-22, CD11c, and DAPI in day 4 colons from C. rodentium infected Rag2^{-/-} mic eMagnification: 400×. FIG. 13(E) depicts data demonstrating that IL-23 directly induces IL-22 production, as measured by ELISA, from isolated murine CD11c⁺ DCs in vitro. All data are representative of two independent experiments.

FIG. 14 depicts data demonstrating that IL-22 can induce STAT3 activation in human colon cells lines. In FIG. 14(A), IL-22^{-/-} mice and wildtype littermates were orally inoculated with 2×10⁹ CFU of C. rodentium. One group of IL-22^{-/-} mice also received mRegIIIγ-Ig fusion protein. Animals were weighed and monitored at the indicated time points. * p<0.05, ** p<0.01. In FIG. 14(B), IL-23 directly induces IL-22 production from isolated human DCs, measured by ELISA. FIG. 14(C) depicts IL-22R expression by FACS on human colon cell lines. FIG. 14(D) depicts a Western blotting showing that IL-22 can induce STAT3 activation in human colon cell lines. FIG. 14(E) depicts a real-time RT-PCR analysis for RegIIIβ and RegIIIγ expression in human colonic epithelial cell lines treated with IL-22. All data are representative of two independent experiments.

FIG. 15 depicts the characterization of anti-IL-22 mAb for immunohistochemistry. FIG. 15(A) depicts colon sections from day 4 C. rodentium infected IL-22^{-/-} and wildtype mice or uninfected wildtype mice, stained with Alexa555 conjugated anti-IL-22 mAb (8E11) or isotype control. FIG. 15(B) depicts cell pellets of IL-22-expressing 293 cells stained with Alexa555 conjugated anti-IL-22 mAb (8E11) or isotype control. The magnification is at 200×.

FIG. 16 depicts a time-course analysis on RegIIIγ and RegIIIβ expression in C57Bl/6 and IL-23 μl 9−/− mouse colons following C. rodentium infection. C57Bl/6 and IL-23p19^{-/-} mice were orally inoculated with 2×10⁹ CFU of C. rodentium. At the indicated time points, mouse colons were collected for RNA extraction and subsequently real-time RT-PCR analysis on mouse RegIIIγ and RegIIIβ expression.

FIG. 17 depicts a time-course analysis on other Reg family members expressions in IL-22−/− and wildtype mouse colons following C. rodentium infection. IL-22−/− and wild type littermates mice were orally inoculated with 2×109 CFU of C. rodentium. At the indicated time points, mouse colons were collected for RNA extraction and subsequently real-time RT-PCR analysis.

FIG. 18 depicts data demonstrating that recombinant human RegIII_fusion protein can partially protect IL-22−/− following C. rodentium infection. IL-22−/− mice and wildtype littermates were orally inoculated with 2×109 CFU of C. rodentium. One group of IL-22−/− mice also received human RegIII_-cFlag fusion proteins. Animals were weighed and monitored at the indicated time points. * p<0.05.

FIG. 19 A-C depicts 161 genes differentially expressed in colon, from IL-22 treatment.

FIG. 20 depicts the 2D hierarchical clustering of 161 genes differentially expressed in colon from IL-22 treatment, where selected genes were clustered by iterative agglomeration of vectors most highly linked by Pearson correlation coefficient, with data for agglomerated vectors summarized by average linkage.

FIG. 21 depicts data demonstrating LTbRFc and anti-IL-22 mAb both lead to mortality after C. rodentium infection.

FIG. 22 depicts data demonstrating LT pathway regulation of multiple upstream aspects involved in IL-22 production.

FIG. 23 depicts data demonstrating IL-22 partially rescues the defects seen in LTbR treated mice.

FIG. 24 depicts data demonstrating anti-IL-22 mAb treatment leads to reduced colon follicles, compromised B/T organization, and reduced DC, T cell and B cell numbers in the colon.

### DETAILED DESCRIPTION OF THE INVENTION

The present invention provides compositions and methods for treatment of microbial disorders by modulation of the host immune response.

The present inventors discovered a novel cytokine pathway that mediates immune response and resistance of mammals to infectious microbial pathogens. In particular, the present inventors discovered that IL-22 is one of the key cytokines that bridges adaptive immune response and innate epithelial defense during early infection of an attaching or effacing (A/E) bacterial pathogen.

As shown herein, cytokines such as IL-22 that are produced by immune cells during the early stages of infection are necessary for intestinal epithelial cells to elicit a full-anti-microbial response and wound-healing response in order to prevent systemic invasion of pathogenic microbes into the host. The studies herein show that IL-22 protects the integrity of the intestinal epithelial barrier and prevents bacterial invasion with systemic spread. Further, the studies herein indicate that IL-22 is involved in the elicitation of the early anti-bacterial IgG responses, and is indespensable for the induction of anti-microbial lectins, such as RegIIIβ and RegIIIγ, from colonic epithelial cells during bacterial infection. The lack of either or both of these mechanisms may contribute to the compromised host defense response with increased systemic spread and mortality in IL-22^{-/-} mice during C. rodentium infection.

As shown herein, the induction of RegIIIβ and RegIIIγ indicates that IL-22 may have broader functions in controlling various bacterial infections. The studies herein further support the role of Th_{IL-17} cells and their effector cytokines in infectious disorders and autoimmune disorders. Further, the studies herein indicate that IL-22 and its downstream products, such as RegIIIβ and RegIIIγ, may be beneficial for the treatment of infectious disorders.

Therefore, the present invention provides methods of treating such microbial disorders by modulation of the host immune response. For example, an anti-microbial immune response in a subject can be enhanced or inhibited by increasing or decreasing an activity of one or more anti-microbial polypeptides (AMPs) that mediate the anti-microbial immune response.

More particularly, the present invention provides AMPs, modulators thereof, and methods of using such compositions for treatment of microbial disorders. Such microbial disorders include, but are not limited to, infectious diseases, for example, EHEC- and EPEC-caused diarrhea, Inflammatory Bowel Disease (IBD) and, more particularly, Ulcerative Colitis (UC) and Crohn's Disease (CD).

All references, including patents, applications, and scientific literature, cited herein are hereby incorporated by reference, in their entirety.

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2. A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology. A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture. Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies. A Practical Approach (D. Catty., ed., IRL Press, 1988-1989); Monoclonal Antibodies. A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### 1. Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

An “anti-microbial polypeptide” or “AMP” is a polypeptide that mediates, or otherwise effects, an anti-microbial immune response to a microbial pathogen, and encompasses encompasses a fragment, variant, analog, derivative or mimetic thereof that retains an AMP activity, e.g., an anti-microbial activity, or activity for modulating an anti-microbial immune response. These methods can be used to treat subjects that are infected with or at risk for infection with an infectious microbial pathogen, e.g., a virus or bacterium. The activity of the AMP can be modulated or differentially regulated (e.g., up or down regulated) relative to another AMP or the same AMP.

An AMP of the present invention encompasses a native AMP and variant forms thereof (which are further defined herein), and may be isolated from a variety of sources, such as from human tissue or from another source, or prepared by recombinant or synthetic methods. A native AMP may be from any species, e.g., murine or human. AMPs of the present invention include, but are not limited to, LT, IL-6, IL-18, IL-22, IL-23 (including e.g., IL-23 p19 or IL-23 p40), and Reg or Reg-related proteins encoded by the genes of the Reg super family. The Reg super family includes Reg and Reg-related genes from human, rat, and mouse and are grouped into four subclasses, types I, II, III, and IV. For example, type I includes human REG Iα, human REG Iβ, rat RegI, and mouse RegI; type II includes mouse RegII; type III includes human REG III, human HIP/PAP (gene expressed in hepatocellular carcinoma-intestine-pancreas/gene encoding pancreatitis-associated protein), rat PAP/Peptide23, rat RegIII/PAPII, rat PAP III, mouse RegIIIα, RegIIIβ, RegIIIγ, mouse RegIIIδ, and hamster INGAP (islet neogenesis-associated protein). Type IV contains human REG IV. Additionally, human Reg-related Sequence (RS) is reportedly a pseudogene. In one embodiment, the REG protein is encoded by a member of the human REG gene family which includes, but is not limited to, REG Iα, REG Iβ, HIP/PAP, REG III, REG IV, and Reg-related sequence (RS).

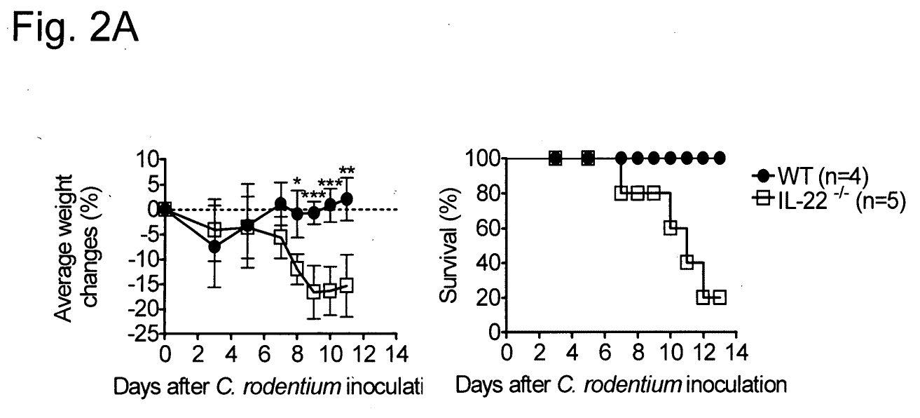

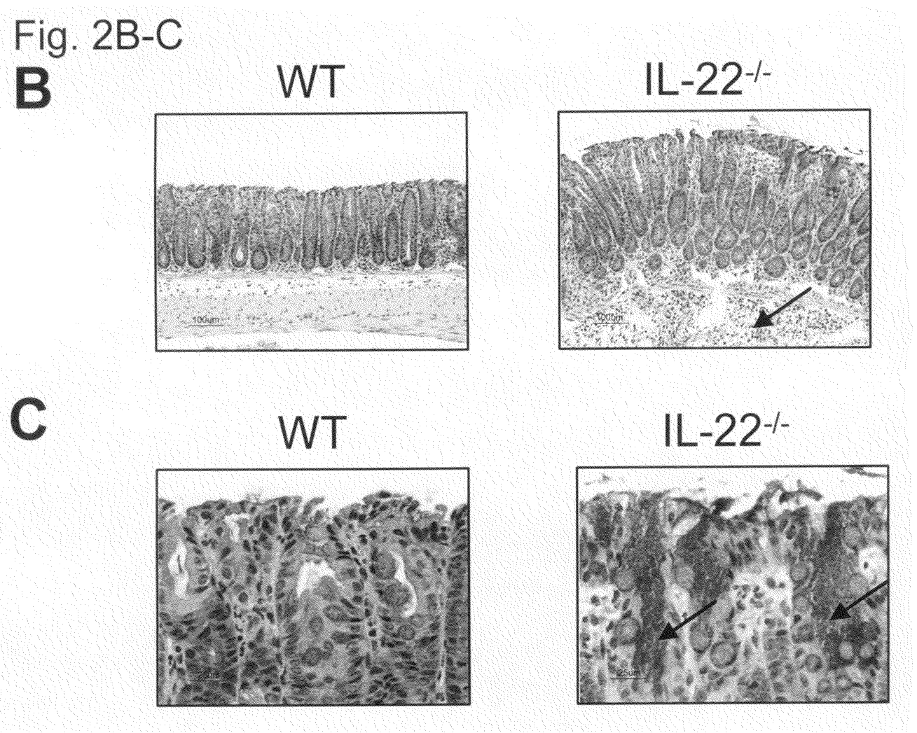

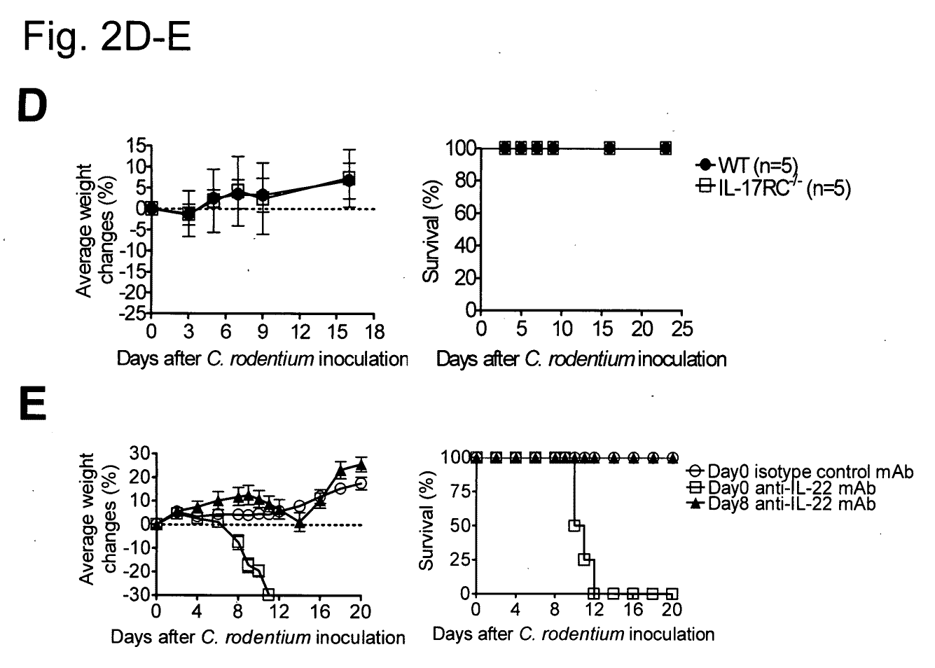

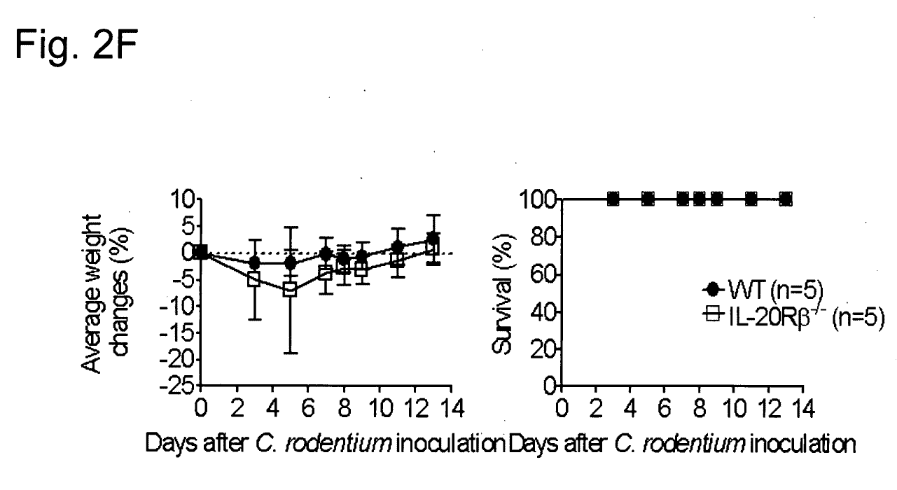

Lymphotoxin (LT) is a trimeric cytokine in the tumor necrosis family; expressed by activated T, B, and NK cells; and involved in inflammatory response signaling and secondary lymphoid organ architecture. “Lymphotoxin-” or “LT” is defined herein as a biologically active polypeptide having the amino acid sequence shown in FIG. 2A of U.S. Pat. No. 5,824,509. “LT” is defined to specifically exclude human TNFα or its natural animal analogues (Pennica et al., Nature 312:20/27 : 724-729 (1984) and Aggarwal et al., J. Biol. Chem. 260: 2345-2354 (1985)). As used herein, “LT” refers to one or more LT subunits as described herein.

“Lymphotoxin-α” or “LTα” is defined to specifically exclude human LTP as defined, for example, in U.S. Pat. No. 5,661,004. “Lymphotoxina-3 trimer” or “LTα3” refers to a homotrimer of LTα monomers. This homotrimer is anchored to the cell surface by the LTβ, transmembrane and cytoplasmic domains.

“Lymphotoxin-αβ” or “LTαβ” or “LTαβ complex” refers to a heterotrimer of LTα with LTβ. These heterotrimers contain either two subunits of LTα and one subunit of LTβ (LTα2β1), or one subunit of LTα and two of LTβ (LTα1β2). The term “LTαβ” or “LTab” as used herein refers to a heterotrimer made up of one subunit of LTα and two of LTβ (LTα1β2).

“Tumor necrosis factor receptor-I” or “TNFR1” and “tumor necrosis factor receptor-II” or “TNFRII” refer to cell-surface TNF receptors for the LTα3 homotrimer, also known as p55 and p75, respectively. “Lymphotoxin-βreceptor” or “LTβ-R” refers to the receptor to which the LTαβ heterotrimers bind.

In some embodiments, the amino acid sequence of an AMP of the present invention comprises an amino acid sequence selected from the following group: SEQ ID NO: 2 (human IL-6), SEQ ID NO: 4 (human IL-12B), SEQ ID NO: 6 (human IL-18), SEQ ID NO: 8 (human IL-22), SEQ ID NO: 10 (human IL-23 p19 or IL-23A), SEQ ID NO: 12 (human REG1A), SEQ ID NO: 14 (human REG1B), SEQ ID NO: 16 (human REG3A, variant 1), SEQ ID NO: 18 (human REG3A, variant 2), SEQ ID NO: 20 (human REG3A, variant 3), SEQ ID NO: 22 (human REG3G, variant 2), SEQ ID NO: 24 (human REG3G, variant 1), SEQ ID NO: 26 (human REG4), SEQ ID NO: 28 (murine IL-6), SEQ ID NO: 30 (murine IL-12B), SEQ ID NO: 32 (murine IL-18), SEQ ID NO: 34 (murine IL-22), SEQ ID NO: 36 (murine IL-23 μl 9 or IL-23A), SEQ ID NO: 38 (murine PAP), SEQ ID NO: 40 (murine REG1), SEQ ID NO: 42 (murine REG2), SEQ ID NO: 44 (murine REG3A), SEQ ID NO: 46 (murine REG3D), SEQ ID NO: 48 (murine REG4), SEQ ID NO: 50 (human LTα), SEQ ID NO: 52 (human LTβ), SEQ ID NO: 54 (murine LTα), and SEQ ID NO: 56 (murine LTβ).

In other embodiments, the nucleic acid sequence encoding an AMP of the present invention comprises a nucleic acid sequence selected from the following group: SEQ ID NO: 1 (human IL-6), SEQ ID NO: 3 (human IL-12B), SEQ ID NO: 5 (human IL-18), SEQ ID NO: 7 (human IL-22), SEQ ID NO: 9 (human IL-23 p19 or IL-23A), SEQ ID NO: 11 (human REG1A), SEQ ID NO: 13 (human REG1B), SEQ ID NO: 15 (human REG3A, variant 1), SEQ ID NO: 17 (human REG3A, variant 2), SEQ ID NO: 19 (human REG3A, variant 3), SEQ ID NO: 21 (human REG3G, variant 2), SEQ ID NO: 23 (human REG3G, variant 1), SEQ ID NO: 25 (human REG4), SEQ ID NO: 27 (murine IL-6), SEQ ID NO: 29 (murine IL-12B), SEQ ID NO: 31 (murine IL-18), SEQ ID NO: 33 (murine IL-22), SEQ ID NO: 35 (murine IL-23 p19 or IL-23A), SEQ ID NO: 37 (murine PAP), SEQ ID NO: 39 (murine REG1), SEQ ID NO: 41 (murine REG2), SEQ ID NO: 43 (murine REG3A), SEQ ID NO: 45 (murine REG3D), SEQ ID NO: 47 (murine REG4), SEQ ID NO: 49 (human LTα), SEQ ID NO: 51 (human LTβ), SEQ ID NO: 53 (murine LTα), and SEQ ID NO: 55 (murine LTβ).

A “native sequence AMP polypeptide” or a “native sequence AMP polypeptide” refers to a polypeptide comprising the same amino acid sequence as a corresponding AMP polypeptide derived from nature. Such native sequence AMP polypeptides can be isolated from nature or can be produced by recombinant or synthetic means. The terms specifically encompass naturally-occurring truncated or secreted forms of the specific AMP polypeptide (e.g., an IL-22 lacking its associated signal peptide), naturally-occurring variant forms (e.g., alternatively spliced forms), and naturally-occurring allelic variants of the polypeptide. In various embodiments of the invention, the native sequence AMP polypeptides disclosed herein are mature or full-length native sequence polypeptides.

A “variant” polypeptide, refers to an active polypeptide having at least about 80% amino acid sequence identity with a full-length native polypeptide sequence. Ordinarily, a variant polypeptide will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity, and alternatively at least about 99% amino acid sequence identity to a full-length or mature native polypeptide sequence.

“Percent (%) amino acid sequence identity,” is defined as the percentage of amino acid residues in a candidate sequence that are identical with the amino acid residues in a specific or reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 1 and 2 below demonstrate how to calculate the % amino acid sequence identity of the amino acid sequence designated “Reference Protein” to the amino acid sequence designated “IL-22”, wherein “IL-22” represents the amino acid sequence of an IL-22 polypeptide of interest, “Reference Protein” represents the amino acid sequence of a polypeptide against which the “IL-22” polypeptide of interest is being compared, and “X, “Y” and “Z” each represent different amino acid residues.

TABLE 1

| | | |
|---|---|---|
| IL-22 | XXXXXXXXXXXXXXX | (Length = 15 amino acids) |
| Reference | XXXXXYYYYYYY | (Length = 12 amino acids) |
| Protein | | |

% amino acid sequence identity = (the number of identically matching amino acid residues between the two polypeptide sequences) divided by (the total number of amino acid residues of the IL-22 polypeptide) = 5 divided by 15 = 33.3%

TABLE 2

| | | |
|---|---|---|
| IL-22 | XXXXXXXXXX | (Length = 10 amino acids) |
| Reference | XXXXXYYYYYYZZYZ | (Length = 15 amino acids) |
| Protein | | |

% amino acid sequence identity = (the number of identically matching amino acid residues between the two polypeptide sequences) divided by (the total number of amino acid residues of the IL-22 polypeptide) = 5 divided by 10 = 50%

An “isolated” biological molecule, such as the various polypeptides, polynucleotides, and antibodies disclosed herein, refers to a biological molecule that has been identified and separated and/or recovered from at least one component of its natural environment.

“Active” or “activity,” with reference to a polypeptide, refers to a biological and/or an immunological activity of a native polypeptide, wherein “biological” activity refers to a biological function of a native polypeptide other than the ability to induce the production of an antibody against an antigenic epitope possessed by the native polypeptide. An “immunological” activity refers to the ability to induce the production of an antibody against an antigenic epitope possessed by a native polypeptide.

The term “antagonist” is used in the broadest sense, and includes any molecule that partially or fully blocks, inhibits, or neutralizes a biological activity of a polypeptide. Also encompassed by “antagonist” are molecules that fully or partially inhibit the transcription or translation of mRNA encoding the polypeptide. Suitable antagonist molecules include, e.g., antagonist antibodies or antibody fragments; fragments or amino acid sequence variants of a native polypeptide; peptides; antisense oligonucleotides; small organic molecules; and nucleic acids that encode polypeptide antagonists or antagonist antibodies. Reference to “an” antagonist encompasses a single antagonist or a combination of two or more different antagonists.

The term “agonist” is used in the broadest sense and includes any molecule that partially or fully mimics a biological activity of a polypeptide, e.g., a native AMP. Also encompassed by “agonist” are molecules that stimulate the transcription or translation of mRNA encoding the polypeptide. Suitable agonist molecules include, e.g., agonist antibodies or antibody fragments; a native polypeptide; fragments or amino acid sequence variants of a native polypeptide; peptides; antisense oligonucleotides; small organic molecules; and nucleic acids that encode polypeptides agonists or antibodies. Reference to “an” agonist encompasses a single agonist or a combination of two or more different agonists.

An “anti-microbial immune response” includes, but is not limited to, resistance or defense to infection by a microbial pathogen. Such resistance or defense can result in an inhibition or decrease in microbial infectivity, replication, proliferation or other activity of a microbial pathogen. In particular, treatment resulting in an anti-microbial immune response can result in the alleviation of a microbial disorder or symptom of a microbial disorder.

“Alleviation”, “alleviating” or equivalents thereof, refers to both therapeutic treatment and prophylactic or preventative measures, wherein the object is to ameliorate, prevent, slow down (lessen), decrease or inhibit the targeted microbial disorder or symptom thereof. Those in need of treatment include those already with the disorder as well as those prone to having the disorder or those in whom the disorder is to be prevented.

With reference to treating a microbial disorder, “treatment”, “treating”, or equivalents thereof, refers to alleviating a microbial disorder or a symptom of a microbial disorder, in a subject having the disorder.

“Chronic” administration refers to administration of an agent(s) in a continuous mode as opposed to an acute mode, so as to maintain the initial therapeutic effect for an extended period of time. “Intermittent” administration is treatment that is not consecutively done without interruption, but rather is cyclic in nature.

“Mammal” for purposes of treatment refers to any animal classified as a mammal, including humans, rodents (e.g., mice and rats), and monkeys; domestic and farm animals; and zoo, sports, laboratory, or pet animals, such as dogs, cats, cattle, horses, sheep, pigs, goats, rabbits, etc. In some embodiments, the mammal is selected from a human, rodent, or monkey. Similarly, “subject” for the purposes of treatment, refers to a mammalian subject, and includes both human and veterinary subjects.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibodies” (Abs) and “immunoglobulins” (Igs) are glycoproteins having similar structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

An antibody that specifically binds to a particular antigen refers to an antibody that is capable of binding the antigen with sufficient affinity such that the antibody is useful as a diagnostic and/or therapeutic agent in targeting the antigen. Preferably, the extent of binding of such an antibody to a non-target polypeptide is less than about 10% of the binding of the antibody to the target antigen as measured, e.g., by a radioimmunoassay (RIA). In certain embodiments, an antibody that binds to a target antigen has a dissociation constant (Kd) of ≦1 μM, ≦100 nM, ≦10 nM, ≦1 nM, or ≦0.1 nM.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region.

“Antibody fragments” comprise only a portion of an intact antibody, wherein the portion retains at least one, and as many as most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO93/1161; Hudson et al. (2003) Nat. Med. 9:129-134; and Hollinger et al., Proc. Natl. Acad. Sci. USA 90: 6444-6448 (1993). Triabodies and tetrabodies are also described in Hudson et al. (2003) Nat. Med. 9:129-134.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256: 495 (1975); Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

An “affinity matured” antibody is one with one or more alterations in one or more HVRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies may be produced by procedures known in the art. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “blocking” antibody, “neutralizing” antibody, or “antagonist” antibody is one which inhibits or reduces a biological activity of the antigen it binds. Such antibodies may substantially or completely inhibit the biological activity of the antigen.

An “agonist antibody,” as used herein, is an antibody which partially or fully mimics a biological activity of a polypeptide of interest.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

In one embodiment, the “Kd” or “Kd value” according to this invention is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (Chen, et al., (1999) J. Mol. Biol. 293:865-881). To establish conditions for the assay, microtiter plates (Dynex) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., (1997) Cancer Res. 57:4593-4599). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% Tween-20 in PBS. When the plates have dried, 150 μl/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays.

According to another embodiment, the Kd or Kd value is measured by surface plasmon resonance assays using a BIAcore™-2000 or a BIAcore™-3000 (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneously fitting the association and dissociation sensorgrams. The equilibrium dissociation constant (Kd) is calculated as the ratio k_{off}/kₒₙ. See, e.g., Chen, Y., et al., (1999) J. Mol. Biol. 293:865-881. If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 mm; emission=340 mm, 16 nm band-pass) at 25° C. of a 20 nM anti-antigen antibody (Fab form) in PBS, pH17.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stirred cuvette.

An “on-rate,” “rate of association,” “association rate,” or “kₒₙ” according to this invention can also be determined as described above using a BLAcore™-2000 or a BIAcore™-3000 system (BIAcore, Inc., Piscataway, N.J.).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to a molecule (such as a nucleic acid, polypeptide, or antibody) so as to generate a “labeled” molecule. The label may be detectable by itself (e.g. radioisotope labels or fluorescent labels) or, in the case of an enzymatic label, may catalyze chemical alteration of a substrate compound or composition, resulting in a detectable product.

By “solid phase” is meant a non-aqueous matrix to which a molecule (such as a nucleic acid, polypeptide, or antibody) can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a nucleic acid, polypeptide, antibody, agonist or antagonist) to a mammal. The components of the liposome are commonly arranged in a bilayer formation, similar to the lipid arrangement of biological membranes.

A “small molecule” or “small organic molecule” is defined herein as an organic molecule having a molecular weight below about 500 Daltons.

An “oligopeptide” that binds to a target polypeptide is an oligopeptide that is capable of binding the target polypeptide with sufficient affinity such that the oligopeptide is useful as a diagnostic and/or therapeutic agent in targeting the polypeptide. In certain embodiments, the extent of binding of an oligopeptide to an unrelated, non-target polypeptide is less than about 10% of the binding of the oligopeptide to the target polypeptide as measured, e.g., by a surface plasmon resonance assay. In certain embodiments, an oligopeptide binds to a target polypeptide with a dissociation constant (Kd) of ≦1 μM, ≦100 mM, ≦10 nM, ≦1 nM, or ≦0.1 nM.

An “organic molecule” that binds to a target polypeptide is an organic molecule other than an oligopeptide or antibody as defined herein that is capable of binding a target polypeptide with sufficient affinity such that the organic molecule is useful as a diagnostic and/or therapeutic agent in targeting the polypeptide. In certain embodiments, the extent of binding of an organic molecule to an unrelated, non-target polypeptide is less than about 10% of the binding of the organic molecule to the target polypeptide as measured, e.g., by a surface plasmon resonance assay. In certain embodiments, an organic molecule binds to a target polypeptide with a dissociation constant (Kd) of ≦1 M, ≦100 nM, ≦10 nM, ≦1 nM, or ≦0.1 nM.

A “biological system” is an in vitro, ex vivo, or in vivo system comprising mammalian cells that share a common signaling pathway.

“Microbial disorder” refers to a disease or condition wherein a microbial pathogen causes, mediates, or otherwise contributes to a morbidity of the disease or condition. Also included are diseases in which stimulation or intervention of an anti-microbial response has an ameliorative effect on progression of the disease. Included within this term are infectious diseases or conditions, and opportunistic diseases resulting from primary infection by a microbial pathogen. Examples of such infectious disease, include, but are not limited to, EHEC- and EPEC-caused diarrhea, Inflammatory Bowel Disease (IBD) and, more particularly, Ulcerative Colitis (UC) and Crohn's Disease (CD).

The term “T cell mediated disease” means a disease in which T cells directly or indirectly mediate or otherwise contribute to a morbidity in a mammal. The T cell mediated disease may be associated with cell mediated effects, lymphokine mediated effects, etc., and even effects associated with B cells if the B cells are stimulated, for example, by the lymphokines secreted by T cells.

An “autoimmune disorder” or “autoimmunity” refers to any condition in which a humoral or cell-mediated immune response is mounted against a body's own tissue. An “IL-23 mediated autoimmune disorder” is any autoimmune disorder that is caused by, maintained, or exacerbated by IL-23 activity.

“Inflammation” refers to the accumulation of leukocytes and the dilation of blood vessels at a site of injury or infection, typically causing pain, swelling, and redness,

“Chronic inflammation” refers to inflammation in which the cause of the inflammation persists and is difficult or impossible to remove.

“Autoimmune inflammation” refers to inflammation associated with an autoimmune disorder.

“Arthritic inflammation” refers to inflammation associated with arthritis.

“Inflammatory bowel disease” or “IBD” refers to a chronic disorder characterized by inflammation of the gastrointestinal tract. IBD encompasses ulcerative colitis, which affects the large intestine and/or rectum, and Crohn's disease, which may affect the entire gastrointestinal system but more commonly affects the small intestine (ileum) and possibly the large intestine.

The term “effective amount” is a concentration or amount of a molecule (e.g., a nucleic acid, polypeptide, agonist, or antagonist) that results in achieving a particular stated purpose. An “effective amount” may be determined empirically. A “therapeutically effective amount” is a concentration or amount of a molecule which is effective for achieving a stated therapeutic effect. This amount may also be determined empirically.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “growth inhibitory agent” when used herein refers to a compound or composition which inhibits growth of a cell, especially a cell overexpressing any of a gene, either in vitro or in vivo. Thus, a growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995), especially p. 13.

The term “cytokine” is a generic term for proteins released by one cell population which act on another cell population as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; lymphotoxin-α and -β, mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12, IL-6, IL-17, IL-18, IL-22, IL-23; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

As used herein, the term “inflammatory cells” designates cells that enhance the inflammatory response such as mononuclear cells, eosinophils, macrophages, and polymorphonuclear neutrophils (PMN).

### II. Compositions and Methods of the Invention

A. Anti-Microbial Polypeptides (AMP) and Modulators Thereof

Anti-microbial polypeptides (AMPs) of the present invention are polypeptides that mediate, or otherwise effect, an anti-microbial immune response to a microbial pathogen. AMPs of the present invention include, but are not limited to, LT, IL-6, IL-22, IL-23 (including e.g., IL-23 p19 or IL-23 p40), and Reg or Reg-related proteins encoded by the genes of the Reg super family. The Reg super family includes Reg and Reg-related genes from human, rat, and mouse and are grouped into four subclasses, types I, II, III, and IV. For example, type I includes human REG Iα, human REG Iβ, rat RegI, and mouse RegI; type II includes mouse RegII; type III includes human REG III, human HIP/PAP (gene expressed in hepatocellular carcinoma-intestine-pancreas/gene encoding pancreatitis-associated protein), rat PAP/Pepticle23, rat RegIII/PAPII, rat PAP III, mouse RegIIIα, RegIIIβ, RegIIIγ, mouse RegIIδ, and hamster INGAP (islet neogenesis-associated protein). Type IV contains human REG IV. Additionally, human Reg-related Sequence (RS) is reportedly a pseudogene. In one embodiment, the REG protein is encoded by a member of the human REG gene family which includes, but is not limited to, REG Iα, REG Iβ, HIP/PAP, REG III, REG IV, and Reg-related sequence (RS).

In some aspects, the amino acid sequence of an AMP of the present invention comprises an amino acid sequence selected from the following group: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, and SEQ ID NO: 56.

In other aspects, the nucleic acid sequence encoding an AMP of the present invention comprises a nucleic acid sequence selected from the following group: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, and SEQ ID NO: 55.

An activity of an AMP of the present invention can be increased or decreased and/or differentially regulated relative to the activity of another AMP or the same AMP. Examples of an activity of an AMP of the present invention, includes, but is not limited to, AMP expression, signal transduction, binding to a binding partner, anti-microbial response, or other biological or immunological activity thereof.

In one embodiment, an increase in the activity of one or more AMPs of the present invention results in an enhanced or induced anti-microbial immune response in a subject.

In one embodiment, AMPs of the present invention include, but are not limited to, polypeptides that directly or indirectly interact with IL-22, e.g., polypeptides that are upstream or downstream of an IL-22 signal transduction pathway that mediates host resistance to infection by a microbial pathogen (e.g., a bacteria or virus). Examples of such AMPs include, but are not limited to, LT, IL-6, IL-18, and IL-23 (including e.g., IL-23 p19 or IL-23 p40).

Modulators of the present invention include, but are not limited to, polypeptides and nucleic acid molecules (e.g., a DNA molecule or RNA molecule) that directly or indirectly modulate an activity of an AMP. Examples of such modulation include, but are not limited to, an increase, decrease, induction or activation, inhibition, or regulation (e.g., up or down regulation) of an activity of an AMP of the present invention.

In a particular embodiment, the modulator indirectly modulates IL-22 activity by decreasing or inhibiting IL-22 Binding Protein (BP) activity and thereby, increasing IL-22 activity. In a further embodiment, the modulator decreases or inhibits binding of IL-22 BP to IL-22 and thereby, increases IL-22 activity.

In some embodiments, the modulator is a polypeptide e.g., a polypeptide that binds to or otherwise interacts with an AMP to increase, induce, or regulate an activity of an AMP. In one embodiment, the modulator is a fusion polypeptide that modulates an activity of an AMP.

In one embodiment, the modulator is an antibody that binds to an AMP. In a particular embodiment, the antibody is a monoclonal antibody. In another embodiment, the antibody is an antibody fragment selected from a Fab, Fab′-SH, Fv, scFv, or (Fab′)₂ fragment. In another embodiment, the antibody is a fusion polypeptide (e.g., an Fc fusion polypeptide). In another embodiment, the antibody is a chimeric antibody. In a particular embodiment, the antibody is humanized. In another embodiment, the antibody is a human antibody. In another embodiment, the antibody binds to the same epitope as an antibody selected from a human, non-human primate, or other mammal (e.g., pig, sheep, rabbit, marmot, rat, or mouse). In a particular embodiment, the antibody is an AMP agonist.

In a particular embodiment, the modulator is a recombinant AMP or nucleic acid molecule encoding an AMP (e.g., a DNA or RNA molecule).

In another particular embodiment, the modulator is a recombinant AMP or nucleic acid molecule encoding an AMP (e.g., a DNA or RNA molecule) that can be expressed in a cell.

AMPs of the present invention encompass native full-length or mature AMPs as well as variants thereof. AMP variants can be prepared by introducing appropriate nucleotide changes into the DNA encoding an AMP, and/or by synthesis of the desired anti-microbial polypeptide. Those skilled in the art will appreciate that amino acid changes may alter post-translational processing of a polypeptide of the present invention, such as changing the number or position of glycosylation sites or altering the membrane anchoring characteristics.

Variations in native AMP or in various domains of the AMP, as described herein, can be made, for example, using any of the techniques and guidelines for conservative and non-conservative mutations set forth, for instance, in U.S. Pat. No. 5,364,934. Variations may be a substitution, deletion or insertion of one or more codons encoding the AMP that results in a change in the amino acid sequence of the AMP as compared with a native sequence AMP. Optionally, the variation is by substitution of at least one amino acid with any other amino acid in one or more domains of the AMP. Guidance in determining which amino acid residue may be inserted, substituted or deleted without adversely affecting the desired activity may be found by comparing the sequence of the AMP with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

In particular embodiments, conservative substitutions of interest are shown in Table 3 under the heading of preferred substitutions. If such substitutions result in a change in biological activity, then more substantial changes, denominated exemplary substitutions in Table 6, or as further described below in reference to amino acid classes, are introduced and the products screened.

TABLE 3

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the AMP polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on cloned DNA to produce a DNA encoding a variant AMP.

Fragments of an AMP or other polypeptides of the present invention are also provided herein. Such fragments may be truncated at the N-terminus or C-terminus, or may lack internal residues, for example, when compared with a full length native protein. Certain fragments lack amino acid residues that are not essential for a desired biological activity of an AMP or polypeptide of the present invention. Accordingly, in certain embodiments, a fragment of an AMP or other polypeptide of the present invention, is biologically active. In certain embodiments, a fragment of full length AMP lacks the N-terminal signal peptide sequence. In certain embodiments, a fragment of full-length AMP is a soluble form of a membrane-bound AMP. For example, a soluble form of AMP may lack all or a substantial portion of the transmembrane domain.

Covalent modifications of AMPs or other polypeptides of the present invention are included within the scope of this invention. One type of covalent modification includes reacting targeted amino acid residues of a polypeptide of the present invention with an organic derivatizing agent that is capable of reacting with selected side chains or the N- or C-terminal residues of the polypeptide. Derivatization with bifunctional agents is useful, for instance, for crosslinking the polypeptide to a water-insoluble support matrix or surface for use in the method for purifying antibodies to the polypeptide, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of a polypeptide of the present invention included within the scope of this invention comprises altering the native glycosylation pattern of the polypeptide. “Altering the native glycosylation pattern” is intended for purposes herein into mean deleting one or more carbohydrate moieties found in the native sequence of a polypeptide of the present invention (either by removing the underlying glycosylation site or by deleting the glycosylation by chemical and/or enzymatic means), and/or adding one or more glycosylation sites that are not present in the native sequence of the polypeptide. In addition, the phrase includes qualitative changes in the glycosylation of the native proteins, involving a change in the nature and proportions of the various carbohydrate moieties present.

A polypeptide of the present invention may also be modified in a way to form a chimeric molecule comprising the polypeptide fused to another, heterologous polypeptide or amino acid sequence. In one embodiment, a chimeric molecule comprises a fusion of the polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tagged polypeptide. Also, provision of the epitope tag enables the AMP to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In another embodiment, a chimeric molecule may comprise a fusion of a polypeptide of the present invention with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble form of a polypeptide of the present invention (e.g., an AMP or polypeptide modulator thereof) in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

1. Preparation of Polypeptides

Polypeptides of the present invention may be prepared by routine recombinant methods, e.g., culturing cells transformed or transfected with a vector containing a nucleic acid encoding an AMP or polypeptide modulator thereof. Host cells comprising any such vector are also provided. By way of example, host cells may be CHO cells, E. coli, or yeast. A process for producing any of the herein described polypeptides is further provided and comprises culturing host cells under conditions suitable for expression of the desired polypeptide and recovering the desired polypeptide from the cell culture.

In other embodiments, the invention provides chimeric molecules comprising any of the herein described polypeptides fused to a heterologous polypeptide or amino acid sequence. Examples of such chimeric molecules include, but are not limited to, any of the herein described polypeptides fused to an epitope tag sequence or an Fc region of an immunoglobulin.

Alternative methods, which are well known in the art, may be employed to prepare a polypeptide of the present invention. For example, a sequence encoding a polypeptide or portion thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of a polypeptide of the present invention or portion thereof may be chemically synthesized separately and combined using chemical or enzymatic methods to produce the full-length polypeptide or portion thereof.

Recombinantly expressed polypeptides of the present invention may be recovered from culture medium or from host cell lysates. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metalchelating columns to bind epitope-tagged forms of a polypeptide of the present invention. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular polypeptide produced. LT polypeptides may be purified by expressing a tagged LT polypeptide such as, for example, an LTα-tagged polypeptide (SEQ ID NO:61).

2. Detection of Gene Expression

Expression of a gene encoding a polypeptide of the present invention can be detected by various methods in the art, e.g., by detecting expression of mRNA encoding the polypeptide. As used herein, the term “detecting” encompasses quantitative or qualitative detection. By detecting gene expression of a polypeptide of the present invention, one can identify, e.g., those tissues that express this gene. Gene expression may be measured using certain methods known to those skilled in the art, e.g., Northern blotting, (Thomas, Proc. Natl. Acad. Sci. USA, 77:5201-5205 [1980]); quantitative PCR; or in situ hybridization, using an appropriately labeled probe, based on the sequences provided herein. Alternatively, gene expression may be measured by immunological methods, such as immunohistochemical staining of tissue sections and assay of cell culture or body fluids, to quantitate directly the expression of gene product. Antibodies useful for immunohistochemical staining and/or assay of sample fluids encompass any of the antibodies provided herein. Conveniently, the antibodies may be prepared against a native sequence encoding e.g., an AMP of the present invention; against a synthetic peptide comprising a fragment of the AMP sequence; or against an exogenous sequence fused to AMP polypeptide or fragment thereof (including a synthetic peptide).

B. Antibodies

Antibodies that bind to any of the above- or below-described polypeptides are provided. In one embodiment, an isolated antibody that binds to an AMP of the present invention and thereby modulates AMP activity, e.g., increasing an activity of the AMP. Exemplary antibodies include polyclonal, monoclonal, humanized, human, bispecific, and heteroconjugate antibodies. An antibody may be an antibody fragment, e.g., a Fab, Fab′-SH, Fv, scFv, or (Fab′)₂ fragment. In one embodiment, an isolated antibody that binds to an IL-22 is provided. In one such embodiment, an antibody partially or completely increases the activity of an AMP of the present invention.

Exemplary monoclonal antibodies that bind an AMP of the present invention are described herein. These antibodies include the anti-IL-22 antibodies designated 3F11.3 (“3F11”), 11H4.4 (“11H4”), and 8E11.9 (“8E11”), and the anti-IL-22R antibodies designated 7E9.10.8 (“7E9”), 8A12.32 (“8A12”), 8H11.32.28 (“8H11”), and 12H5. In one embodiment, a hybridoma that produces any of those antibodies is provided. In one embodiment, monoclonal antibodies that compete with 3F11.3, 11H4.4, or 8E11.9 for binding to IL-22 are provided. In another embodiment, monoclonal antibodies that bind to the same epitope as 3F11.3, 11H4.4, or 8E11.9 are provided. In another embodiment, monoclonal antibodies that compete with 7E9, 8A12, 8H11, or 12H5 for binding to IL-22R are provided. In one embodiment, monoclonal antibodies that bind to the same epitope as 7E9, 8A12, 8H11, or 12H5 are provided. Various embodiments of antibodies are provided below:

1. Polyclonal Antibodies

Antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the polypeptide of interest or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

Antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the polypeptide of interest or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies that bind to the polypeptide of interest. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. (2001) in Methods in Molecular Biology 178:1-37 (O'Brien et al., ed., Human Press, Totowa, N.J.), and in certain embodiments, in Lee et al. (2004) J. Mol. Biol. 340:1073-1093.

In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the antibodies of the invention can be obtained by designing a suitable antigen screening procedure to select for the phage clone of interest followed by construction of a full length antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

3. Monovalent Antibodies

Monovalent antibodies are also provided. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

4. Antibody Fragments

Antibody fragments are also provided. Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to solid tumors. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody. phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

5. Humanized Antibodies

Humanized antibodies are also provided. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

6. Human Antibodies

Human antibodies are also provided. Human antibodies can be constructed by combining Fv clone variable domain sequence(s) selected from human-derived phage display libraries with known human constant domain sequences(s) as described above. Alternatively, human monoclonal antibodies of the invention can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is now possible to produce transgenic animals (e.g. mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255 (1993); Bruggermann et al., Year in Immunol., 7: 33 (1993).

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

7. Bispecific Antibodies

Bispecific antibodies are also provided. Bispecific antibodies are monoclonal antibodies that have binding specificities for at least two different antigens. In certain embodiments, bispecific antibodies are human or humanized antibodies. In certain embodiments, one of the binding specificities is for a polypeptide of interest and the other is for any other antigen. In certain embodiments, bispecific antibodies may bind to two different epitopes of a polypeptide of interest. Bispecific antibodies may also be used to localize cytotoxic agents to cells which express a polypeptide of interest, such a cell surface polypeptide. These antibodies possess a TAT226-binding arm and an arm which binds a cytotoxic agent, such as, e.g., saporin, anti-interferon-α, vinca alkaloid, ricin A chain, methotrexate or radioactive isotope hapten. Bispecific antibodies can be prepared as full length antibodies or antibody fragments (e.g. F(ab′)₂ bispecific antibodies).

Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature, 305: 537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J, 10: 3655 (1991).

According to a different approach, antibody variable domains with the desired binding specificities (antibody-antigen combining sites) are fused to immunoglobulin constant domain sequences. The fusion, for example, is with an immunoglobulin heavy chain constant domain, comprising at least part of the hinge, CH2, and CH3 regions. In certain embodiments, the first heavy-chain constant region (CH1), containing the site necessary for light chain binding, is present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach, the interface between a pair of antibody molecules can be engineered to maximize the percentage of heterodimers which are recovered from recombinant cell culture. The interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/00373, and EP 03089). Heteroconjugate antibodies may be made using any convenient cross-linking method. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the HER2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

8. Multivalent Antibodies

Multivalent antibodies are also provided. A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino-terminal to the Fc region. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (for example, two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (for example, four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

9. Single-Domain Antibodies

Single-domain antibodies are also provided. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). In one embodiment, a single-domain antibody consists of all or a portion of the heavy chain variable domain of an antibody.

10. Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, an antibody of the invention is altered to increase or decrease the extent to which the antibody is glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that one or more of the above-described tripeptide sequences (for N-linked glycosylation sites) is created or removed. The alteration may also be made by the addition, deletion, or substitution of one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

In certain embodiments, a glycosylation variant comprises an Fe region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 1), and knockout cell lines, such as alpha-1,6-fucosyltransferase gene, FU78, knockout CHO cells (Yamane-Ohnuki et al. Biotech. Bioeng. 87: 614 (2004)).

Another type of variant is an amino acid substitution variant. These variants have at least one amino acid residue in the antibody molecule replaced by a different residue. Sites of interest for substitutional mutagenesis include the hypervariable regions, but FR alterations are also contemplated. Conservative substitutions are shown in Table 3 above under the heading of “preferred substitutions.” If such substitutions result in a desirable change in biological activity, then more substantial changes, denominated “exemplary substitutions” in Table 3, or as further described above in reference to amino acid classes, may be introduced and the resulting antibodies screened for the desired binding properties.

One type of substitutional variant involves substituting one or more hypervariable region residues of a parent antibody (e.g. a humanized or human antibody). Generally, the resulting variant(s) selected for further development will have modified (e.g., improved) biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g. binding affinity). In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such variants are generated, the panel of variants is subjected to screening using techniques known in the art, including those described herein, and antibodies with superior properties in one or more relevant assays may be selected, for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of antibodies of the invention, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In accordance with this description and the teachings of the art, it is contemplated that in some embodiments, an antibody of the invention may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter Nature 322:738-40 (1988); U.S. Pat. No. 5,648,260; U.S. Pat. No. 5,624,821; and WO94/29351 concerning other examples of Fc region variants. WO00/42072 (Presta) and WO 2004/056312 (Lowman) describe antibody variants with improved or diminished binding to FcRs. The content of these patent publications are specifically incorporated herein by reference. See, also, Shields et al. J. Biol. Chem. 9(2): 6591-6604 (2001). Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). These antibodies comprise an Fc region with one or more substitutions therein which improve binding of the Fc region to FcRn. Polypeptide variants with altered Fc region amino acid sequences and increased or decreased C1q binding capability are described in U.S. Pat. No. 6,194,551B1, WO99/51642. The contents of those patent publications are specifically incorporated herein by reference. See, also, Idusogie et al. J. Immunol. 164: 4178-4184 (2000).

In one embodiment, the invention provides antibodies comprising modifications in the interface of Fc polypeptides comprising the Fc region, wherein the modifications facilitate and/or promote heterodimerization. These modifications comprise introduction of a protuberance into a first Fc polypeptide and a cavity into a second Fc polypeptide, wherein the protuberance is positionable in the cavity so as to promote complexing of the first and second Fc polypeptides. Methods of generating antibodies with these modifications are known in the art, e.g., as described in U.S. Pat. No. 5,731,168.

11. Antibody Derivatives

Antibodies can be further modified to contain additional nonproteinaceous moieties that are known in the art and readily available. Preferably, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

In certain embodiments, an antibody may be labeled and/or may be immobilized on a solid support. In a further embodiment, an antibody is an anti-idiotypic antibody.

12. Heteroconjugate Antibodies

Heteroconjugate antibodies are also provided. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

13. Effector Function Engineering

It may be desirable to modify an antibody with respect to effector function, so as to enhance, e.g., the effectiveness of the antibody in treating a microbial disorder. For example, cysteine residue(s) may be introduced into the Fc region, thereby allowing interchain disulfide bond formation in this region. Homodimeric antibodies with enhanced anti-anti-microbial activity may also be prepared using heterobifunctional cross-linkers. Alternatively, an antibody can be engineered that has dual Fc regions and may thereby have enhanced activity.

14. Vectors, Host Cells, and Recombinant Methods

For recombinant production of an antibody, in one embodiment, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

a) Generating Antibodies Using Prokaryotic Host Cells:

(1) Vector Construction

Polynucleotide sequences encoding polypeptide components of an antibody can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell may be used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

An expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one embodiment of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another embodiment, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun Gene, 159:203 (1995).

Antibodies of the invention can also be produced by using an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence. In certain embodiments, changes in the nucleotide sequence are silent. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

In one embodiment, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the invention.

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-121.9; AICC Deposit No. 27,325) and derivatives thereof, including strain 33D3 having genotype W3110 ΔfhuA (ΔtonA) ptr3 lac lq lacL8 ΔompTΔ(nmpc-fepE) degP41 kanR (U.S. Pat. No. 5,639,635). Other strains and derivatives thereof, such as E. coli 294 (ATCC 31,446), E. coli B, E. coliλ 1776 (ATCC 31,537) and E. coli RV308(ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

(2) Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. In certain embodiments, for E. coli growth, growth temperatures range from about 20° C. to about 39° C.; from about 25° C. to about 37° C.; or about 30° C. The pH of the medium may be any pH ranging from about 5 to about 9, depending mainly on the host organism. In certain embodiments, for E. coli, the pH is from about 6.8 to about 7.4, or about 7.0.

If an inducible promoter is used in the expression vector of the invention, protein expression is induced under conditions suitable for the activation of the promoter. In one embodiment of the invention, PhoA promoters are used for controlling transcription of the polypeptides. Accordingly, the transformed host cells are cultured in a phosphate-limiting medium for induction. In certain embodiments, the phosphate-limiting medium is the C.R.A.P. medium (see, e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one embodiment of the invention, antibody production is conducted in large quantity by a fermentation process. Various large-scale fed-batch fermentation procedures are available for production of recombinant proteins. Large-scale fermentations have at least 1000 liters of capacity, and in certain embodiments, about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J. Biol. Chem. 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

(3) Antibody Purification

In one embodiment, an antibody produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one embodiment, Protein A immobilized on a solid phase is used for immunoaffinity purification of the antibody products of the invention. Protein A is a 41 kD cell wall protein from Staphylococcus aureas which binds with a high affinity to the Fc region of antibodies. Lindmark et al (1983) J. Immunol. Meth. 62:1-13. The solid phase to which Protein A is immobilized can be a column comprising a glass or silica surface, or a controlled pore glass column or a silicic acid column. In some applications, the column is coated with a reagent, such as glycerol, to possibly prevent nonspecific adherence of contaminants.

As the first step of purification, a preparation derived from the cell culture as described above can be applied onto a Protein A immobilized solid phase to allow specific binding of the antibody of interest to Protein A. The solid phase would then be washed to remove contaminants non-specifically bound to the solid phase. Finally the antibody of interest is recovered from the solid phase by elution.

b) Generating Antibodies Using Eukaryotic Host Cells:

A vector for use in a eukaryotic host cell generally includes one or more of the following non-limiting components: a signal sequence, an origin of replication, one or more marker genes, an enhancer element, a promoter, and a transcription termination sequence.

(1) Signal Sequence Component

A vector for use in a eukaryotic host cell may also contain a signal sequence or other polypeptide having a specific cleavage site at the N-terminus of the mature protein or polypeptide of interest. The heterologous signal sequence selected may be one that is recognized and processed (i.e., cleaved by a signal peptidase) by the host cell. In mammalian cell expression, mammalian signal sequences as well as viral secretory leaders, for example, the herpes simplex gD signal, are available. The DNA for such a precursor region is ligated in reading frame to DNA encoding the antibody.

(2) Origin of Replication

Generally, an origin of replication component is not needed for mammalian expression vectors. For example, the SV40 origin may typically be used only because it contains the early promoter.

(3) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

For example, in some embodiments, cells transformed with the DHFR selection gene are first identified by culturing all of the transformants in a culture medium that contains methotrexate (Mtx), a competitive antagonist of DHFR. In some embodiments, an appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

(4) Promoter Component

Expression and cloning vectors usually contain a promoter that is recognized by the host organism and is operably linked to nucleic acid encoding a polypeptide of interest (e.g., an antibody). Promoter sequences are known for eukaryotes. For example, virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. In certain embodiments, any or all of these sequences may be suitably inserted into eukaryotic expression vectors.

Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982), describing expression of human β-interferon cDNA in mouse cells under the control of a thymidine kinase promoter from herpes simplex virus. Alternatively, the Rous Sarcoma Virus long terminal repeat can be used as the promoter.

(5) Enhancer Element Component

Transcription of DNA encoding an antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) describing enhancer elements for activation of eukaryotic promoters. The enhancer may be spliced into the vector at a position 5′ or 3′ to the antibody polypeptide-encoding sequence, but is generally located at a site 5′ from the promoter.

(6) Transcription Termination Component

Expression vectors used in eukaryotic host cells may also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

(7) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI 38, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(8) Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCN™ drug), trace elements (defined as inorganic compounds usually present at final concentrations in the micromolar range), and glucose or an equivalent energy source. Any other supplements may also be included at appropriate concentrations that would be known to those skilled in the art. The culture conditions, such as temperature, pH, and the like, are those previously used with the host cell selected for expression, and will be apparent to the ordinarily skilled artisan.

(9) Purification of Antibody

When using recombinant techniques, the antibody can be produced intracellularly, or directly secreted into the medium. If the antibody is produced intracellularly, as a first step, the particulate debris, either host cells or lysed fragments, may be removed, for example, by centrifugation or ultrafiltration. Where the antibody is secreted into the medium, supernatants from such expression systems may be first concentrated using a commercially available protein concentration filter, for example, an Amicon or Millipore Pellicon ultrafiltration unit. A protease inhibitor such as PMSF may be included in any of the foregoing steps to inhibit proteolysis, and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a convenient technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Methods 62:1-13 (1983)). Protein G is recommended for all mouse isotypes and for human γ3 (Guss et al., EMBO J. 5:15671575 (1986)). The matrix to which the affinity ligand is attached may be agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to further purification, for example, by low pH hydrophobic interaction chromatography using an elution buffer at a pH between about 2.5-4.5, preferably performed at low salt concentrations (e.g., from about 0-0.25M salt).

In general, various methodologies for preparing antibodies for use in research, testing, and clinical use are well-established in the art, consistent with the above-described methodologies and/or as deemed appropriate by one skilled in the art for a particular antibody of interest.

C. Agonists and Antagonists

Agonists and antagonists of an AMP of the present inventions are provided. Such AMP modulators are encompassed in the present invention and useful for treating a microbial disorder as provided herein.

In one embodiment, an agonist or antagonist of an AMP of the present invention is an antibody, e.g., and IL-22 antibody or an anti-IL-22R antibody. In certain embodiments, an anti-IL-22 antibody is an agonistic antibody that promotes the interaction of IL-22 with IL-22R. In another embodiment, an anti-IL-22 antibody is an antagonistic antibody that fully or partially blocks the interaction of IL-22 with IL-22R. In certain embodiments, an anti-IL-22R antibody binds to the extracellular ligand binding domain of an IL-22R. For example, an anti-IL-22R antibody may bind to the extracellular ligand binding domain of human IL-22R, which is found in SEQ ID NO:3 from about amino acids 18-228.

In a particular embodiment, an IL-22 agonist is an antibody that binds IL-22BP and blocks or inhibits binding of IL-22BP to IL-22, and thereby induces or increases an IL-22 activity (e.g., binding to IL-22R).

In another embodiment, an agonist or antagonist of an AMP of the present invention is an oligopeptide that binds to the AMP. In one embodiment, an oligopeptide binds to the extracellular ligand binding domain of IL-22R. Oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length. Such oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. USA, 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In yet another embodiment, an agonist or antagonist of an AMP of the present invention is an organic molecule that binds to the AMP, other than an oligopeptide or antibody as described herein. An organic molecule may be, for example, a small molecule. In one embodiment, an organic molecule binds to the extracellular domain of an IL-22R. An organic molecule that binds to an AMP of the present invention may be identified and chemically synthesized using known methodology (see, e.g., PCT Publication Nos. WO00/00823 and WO00/39585). Such organic molecules are usually less than about 2000 daltons in size, alternatively less than about 1500, 750, 500, 250 or 200 daltons in size, wherein such organic molecules that are capable of binding to an AMP of the present invention may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585).

In a particular embodiment, an IL-22 agonist is an organic molecule that binds IL-22BP and blocks or inhibits binding of IL-22BP to IL-22, and thereby induces or increases an IL-22 activity (e.g., binding to IL-22R).

In a particular embodiment, an IL-22 antagonist is a soluble IL-22 receptor, e.g., a form of IL-22R that is not membrane bound. Such soluble forms of IL-22R may compete with membrane-bound IL-22R for binding to IL-22. In certain embodiments, a soluble form of IL-22R may comprise all or a ligand-binding portion of an extracellular domain of IL-22R, e.g., all or a ligand-binding portion of a polypeptide comprising amino acids 18-228 of SEQ ID NO:3. In certain embodiments, a soluble form of IL-22R lacks a transmembrane domain. For example, a soluble form of human IL-22R may lack all or a substantial portion of the transmembrane domain from about amino acids 229-251 of SEQ ID NO:3.

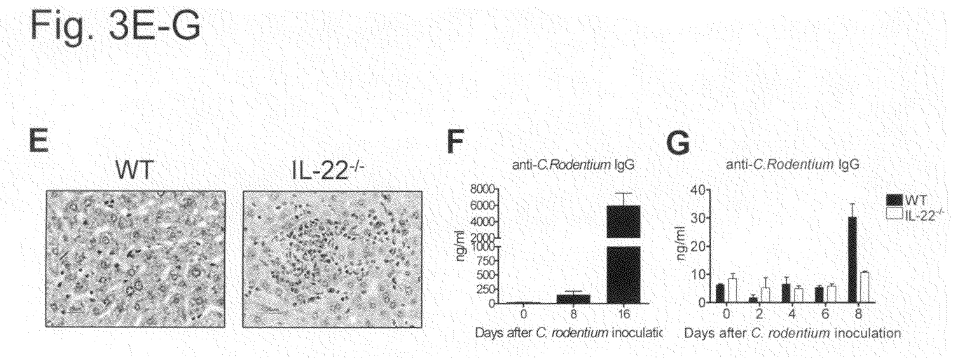

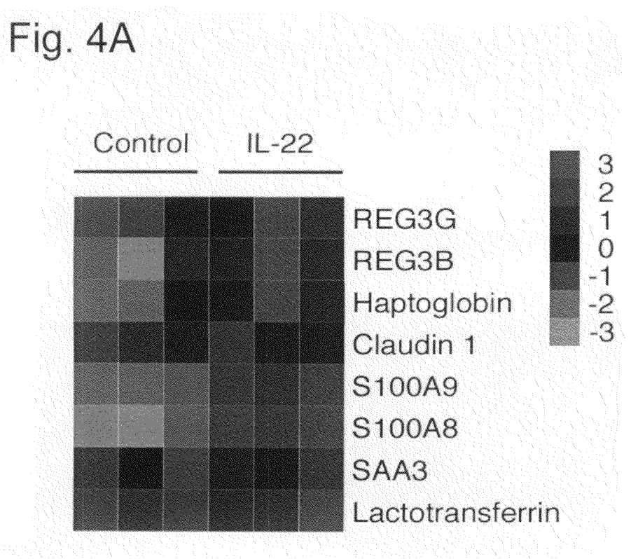

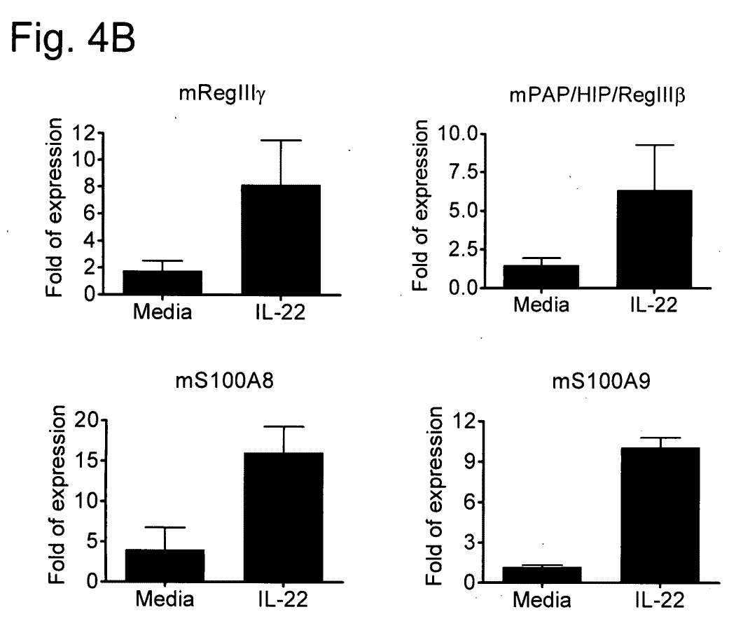

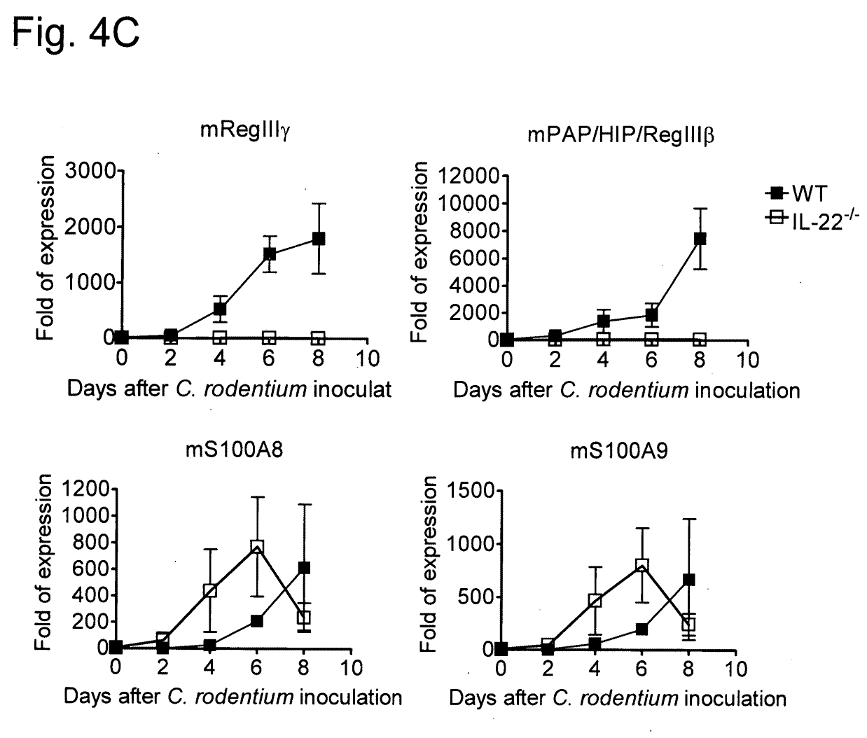

A naturally occurring, soluble receptor for IL-22 has been reported. See Dumoutier L. et al., “Cloning and characterization of IL-22 binding protein, a natural antagonist of IL-10-related T cell-derived inducible factor/IL-22,” J. Immunol. 166:7090-7095 (2001); and Xu W. et al., “A soluble class II cytokine receptor, IL-22RA2, is a naturally occurring IL-22 antagonist,” Proc. Natl. Acad. Sci. U.S.A. 98:9511-9516 (2001). That receptor is variously designated “WL-22BP” or “IL-22RA2” in the art. The sequence of a human IL-22BP is shown in FIG. 4. The term “IL-22BP” or “IL-22 binding protein” as used herein refers to any native IL-22BP from any vertebrate source, including mammals such as primates (e.g. humans and monkeys) and rodents (e.g., mice and rats), unless otherwise indicated.

In yet another embodiment, an antagonist of IL-22 is an antisense nucleic acid that decreases expression of the IL-22 or IL-22R gene (i.e., that decreases transcription of the IL-22 or IL-22R gene and/or translation of IL-22 or IL-22R mRNA). In certain embodiments, an antisense nucleic acid binds to a nucleic acid (DNA or RNA) encoding IL-22 or IL-22R. In certain embodiments, an antisense nucleic acid is an oligonucleotide of about 10-30 nucleotides in length (including all points between those endpoints). In certain embodiments, an antisense oligonucleotide comprises a modified sugar-phosphodiester backbones (or other sugar linkages, including phosphorothioate linkages and linkages as described in WO 91/06629), wherein such modified sugar-phosphodiester backbones are resistant to endogenous nucleases. In one embodiment, an antisense nucleic acid is an oligodeoxyribonucleotide, which results in the degradation and/or reduced transcription or translation of mRNA encoding IL-22 or IL-22R. In certain embodiments, an antisense nucleic acid is an RNA that reduces expression of a target nucleic acid by “RNA interference” (“RNAi”). For review of RNAi, see, e.g., Novina et al. (2004) Nature 430:161-164. Such RNAs are derived from, for example, short interfering RNAs (siRNAs) and microRNAs. siRNAs, e.g., may be synthesized as double stranded oligoribonucleotides of about 18-26 nucleotides in length. Id.

In yet another embodiment, agonists of IL-22 are provided. Exemplary agonists include, but are not limited to, native IL-22 or IL-22R; fragments, variants, or modified forms of IL-22 or IL-22R that retain at least one activity of the native polypeptide; agents that are able to bind to and activate IL-22R; and agents that induce overexpression of IL-22 or IL-22R or nucleic acids encoding IL-22 or IL-22R.

D. Pharmaceutical Formulations

The invention provides pharmaceutical formulations. In one embodiment, a pharmaceutical formulation comprises 1) an active agent, e.g., any of the above-described polypeptides, antibodies, agonists, or antagonists; and 2) a pharmaceutically acceptable carrier. In a further embodiment, a pharmaceutical formulation further comprises at least one additional therapeutic agent.

Pharmaceutical formulations are prepared for storage by mixing an agent having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Lipofections or liposomes can also be used to deliver an agent into a cell. Where the agent is an antibody fragment, the smallest inhibitory fragment which specifically binds to the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Natl. Acad. Sci. USA 90, 7889-7893 [1993]). Antibodies disclosed herein may also be formulated as immunoliposomes. Liposomes containing an antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of an antibody of the present invention can be conjugated to liposomes as described in Martin et al., J. Biol. Chem., 257: 286-288 (1982) via a disulfide-interchange reaction. A chemotherapeutic agent (such as doxorubicin) is optionally contained within the liposome. See Gabizon et al., J. National Cancer Inst., 81(19): 1484 (1989).

An agent may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of an agent may be prepared. Suitable examples of sustained-release preparations include semipermeable matrices of solid hydrophobic polymers containing the agent, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

A pharmaceutical formulation herein may also contain more than one active compound as necessary for the particular indication being treated. For example, in one embodiment, a pharmaceutical formulation containing more than one active compound comprises 1) at least one agonist of IL-22, e.g., antibody that binds to IL-22 and/or an antibody that binds to IL-22R; and 2) at least one antibody that binds to IL-6 or IL-23 (wherein any number of the antibodies listed in 2) may be selected in any combination). In another embodiment, a pharmaceutical formulation contains two or more active compounds having complementary activities.

E. Methods of Treatment

The present invention further provides methods of treating a microbial disorder. In another embodiment, the present invention provides a method of treating a microbial disorder, in a subject, comprising administering to the subject an effective amount of pharmaceutical composition comprising an AMP or modulator of the AMP, wherein the AMP is selected from a group consisting of: LT, IL-6, IL-18, IL-22, IL-23, REG Iα, REG Iβ, HIP/PAP, REG III, REG IV and Reg-related sequence (RS). In one embodiment the disorder is EHEC- or EPEC-caused diarrhea, Inflammatory Bowel Disease (IBD) or, more particularly, Ulcerative Colitis (UC) and Crohn's Disease (CD).

In one embodiment, the present invention provides a method of treating an infection by a microbial pathogen (e.g., a bacteria or virus), in a subject, comprising administering to the subject an effective amount of pharmaceutical composition comprising an AMP or modulator of the AMP, wherein the AMP is selected from a group consisting of: LT, IL-6, IL-18, IL-22, IL-23, REG Iα, REG Iβ, HIP/PAP, REG III, REG IV and Reg-related sequence (RS).

In another embodiment, the present invention provides a method of modulating the activity of an AMP in cells of a subject infected with a microbial pathogen (e.g., a bacteria or virus), comprising contacting the cells with an AMP or modulator of the AMP, wherein the AMP is selected from a group consisting of: LT, IL-6, IL-18, IL-22, IL-23, REG Iα, REG Iβ, HIP/PAP, REG III (e.g., REG IIIβ or REGIIIγ), REG IV, and Reg-related sequence (RS).

In another embodiment, the present invention provides a method of treating a microbial disorder, in a subject, comprising contacting cells of the subject with a nucleic acid molecule (e.g., a DNA or RNA molecule) encoding an AMP or modulator of the AMP, wherein the AMP is selected from a group consisting of: LT, IL-6, IL-18, IL-22, L-23, REG Iα, REG Iβ, HIP/PAP, REG III, REG IV and Reg-related sequence (RS). In one embodiment the disorder is EHEC- or EPEC-caused diarrhea, Inflammatory Bowel Disease (IBD) or, more particularly, Ulcerative Colitis (UC) or Crohn's Disease (CD).

In another embodiment, the present invention provides a method of modulating the activity of an AMP in cells of a subject infected with a microbial pathogen (e.g., a bacteria or virus), comprising contacting the cells with a nucleic acid molecule (e.g., a DNA or RNA molecule) encoding an AMP or modulator of the AMP, wherein the AMP is selected from a group consisting of: LT, IL-6, IL-18, IL-22, IL-23, REG Iα, REG Iβ, HIP/PAP, REG III (e.g., REG IIIβ or REGIIIγ), REG IV, and Reg-related sequence (RS).

Examples of a microbial pathogen include, but are not limited to, a bacteria or virus. In one embodiment, the microbial pathogen is a bacteria e.g., a gram-negative or gram-positive bacteria. In a particular embodiment, the bacteria is a gram-negative bacteria. In another embodiment, the bacteria is an attaching or effacing (A/E) bacteria and, more particularly, an enterohemorrhagic Escherichia coli (EHEC) or enteropathogenic E. Coli (EPEC). In one embodiment, the bacteria is enteropathogenic E. coli (EHEC) is E. coli O₁₅₇:H7 or E. coli 055:H7.

The therapeutic methods of the present invention comprise one or more compositions or pharmaceutical formulations of the present invention. Such methods include in vitro, ex vivo, and in vivo therapeutic methods, unless otherwise indicated.

In various embodiments, the present invention provides methods of modulating an anti-microbial immune response by stimulating or inhibiting an AMP-mediated signaling pathway and/or Th_{IL-17} cell function. Such methods are useful for treatment of microbial disorders. For example, in one embodiment, the present invention provides a method of enhancing an anti-microbial immune response by stimulating an AMP-mediated signaling pathway, e.g., and IL-22 and/or IL-23 mediated signaling pathway. In another embodiment, the present invention provides methods of modulating an anti-microbial immune response by stimulating or inhibiting a cytokine-mediated signaling pathway. For example, in one embodiment, the present invention provides methods of enhancing an anti-microbial immune response by stimulating a cytokine-mediated signaling pathway, e.g., an IL-22 and/or IL-23 signaling pathway. Moreover, the present invention provides methods of modulating an anti-microbial immune response by stimulating or inhibiting a Th_{IL-17} cell function.

In one embodiment, the present invention provides a method of stimulating an AMP-mediated signaling pathway in a biological system, the method comprising providing an AMP agonist to the biological system. Examples of such a biological system include, but are not limited to, mammalian cells in an in vitro cell culture system or in an organism in vivo. In another embodiment, the present invention provides a method of inhibiting an AMP-mediated signaling pathway in a biological system, the method comprising providing an AMP antagonist to the biological system.

In a particular embodiment, the present invention provides a method of enhancing an anti-microbial immune response in a biological system by stimulating an IL-23 and/or IL-22 mediated signaling pathway in a biological system, the method comprising providing an IL-22 or IL-22 agonist to the biological system. In one embodiment, an IL-22 agonist is IL-22. In another embodiment, the IL-22 agonist is an antibody that binds to IL-22.

In another embodiment, a method of inhibiting an IL-23-mediated signaling pathway in a biological system is provided, the method comprising providing an IL-22 antagonist to the biological system. In one embodiment, the antagonist of IL-22 is an antibody, e.g., a neutralizing anti-IL-22 antibody and/or a neutralizing anti-IL-22R antibody.

In another embodiment, the present invention provides a method of stimulating a Th_{IL-17} cell, function, the method comprising exposing a Th_{IL-17} cell to an agonist of an AMP that mediates the IL-23 mediated signaling pathway (e.g., IL-23, IL-6, or IL-22). Such methods are useful for treating a microbial disorder. In one embodiment, an IL-22 agonist is IL-22. In another embodiment, the IL-22 agonist is an antibody that binds to IL-22.

In another embodiment, a method of inhibiting a Th_{IL-17} cell function is provided, the method comprising exposing a Th_{IL-17} cell to an antagonist of an AMP that mediates the IL-23 mediated signaling pathway (e.g., 1′-23, IL-6, or IL-22). In one embodiment the antagonist is an anti-IL-22 antibody, e.g., a neutralizing anti-IL-22 antibody.

Exemplary Th_{IL-17} cell functions include, but are not limited to, stimulation of cell-mediated immunity (delayed-type hypersensitivity); recruitment of innate immune cells, such as myeloid cells (e.g., monocytes and neutrophils) to sites of inflammation; and stimulation of inflammatory cell infiltration into tissues. In one embodiment, a Th_{IL-17} cell function is mediated by IL-23 and/or IL-22.

Compositions of the present invention are administered to a mammal, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation (intranasal, intrapulmonary) routes. Intravenous or inhaled administration of polypeptides and antibodies is preferred.

For the treatment or reduction in the severity of a microbial disorder, the appropriate dosage of a composition of the invention will depend on the type of disorder to be treated, as defined above, the severity and course of the disorder, whether the agent is administered for preventive or therapeutic purposes, previous therapy, the patient's clinical history and response to the compound, and the discretion of the attending physician. The compound is suitably administered to the patient at one time or over a series of treatments.

For example, depending on the type and severity of a disorder, about 1 μg/kg to 15 mg/kg (e.g., 0.1-20 mg/kg) of a polypeptide or antibody is an initial candidate dosage for administration to a patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

F. Diagnostic Methods and Methods of Detection

In one embodiment, the present invention provides a method of detecting the presence of an AMP in a biological sample, comprising contacting the biological sample with an antibody to the AMP, under conditions permissive for binding of the antibody to the AMP, and detecting whether a complex is formed between the antibody and AMP.

In one embodiment, the present invention provides a method of monitoring treatment of a microbial disorder in a subject, wherein the method comprises detecting the level of expression of a gene encoding an AMP in a test sample of tissue cells obtained from the subject in need of treatment, and the expression level in the test sample is detected. The detection may be qualitative or quantitative. In one embodiment, the test sample comprises blood or serum. In one embodiment, detecting the level of expression of a gene encoding an AMP comprises (a) contacting an anti-AMP antibody with a test sample obtained from the mammal, and (b) detecting the formation of a complex between the antibody and an AMP in the test sample. The antibody may be linked to a detectable label. Complex formation can be monitored, for example, by light microscopy, flow cytometry, fluorimetry, or other techniques known in the art. The test sample may be obtained from an individual suspected of having a microbial disorder.

In one embodiment, detecting the level of expression of a gene encoding an AMP polypeptide comprises detecting the level of mRNA transcription from the gene. Levels of mRNA transcription may be detected, either quantitatively or qualitatively, by various methods known to those skilled in the art. Levels of mRNA transcription may also be detected directly or indirectly by detecting levels of cDNA generated from the mRNA. Exemplary methods for detecting levels of mRNA transcription include, but are not limited to, real-time quantitative RT-PCR and hybridization-based assays, including microarray-based assays and filter-based assays such as Northern blots.

In another embodiment, the present invention provides a method of detecting the presence of an AMP in a biological sample, comprising contacting the biological sample with an antibody to the AMP, under conditions permissive for binding of the antibody to the AMP, and detecting whether a complex is formed between the antibody and AMP.

In another embodiment, the present invention concerns a diagnostic kit containing an anti-AMP in suitable packaging. The kit preferably contains instructions for using the antibody to detect an AMP. In one embodiment, the diagnostic kit is for diagnosing a microbial disorder. In one embodiment, the diagnostic kit is for diagnosing a microbial infection.

In another embodiment, the present invention provides a kit comprising one or more AMPs of the present invention and/or modulators thereof. In another embodiment, the present invention provides a kit comprising one or more one or more pharmaceutical compositions each comprising an AMP of the present invention or modulator thereof.

G. Assays

1. Cell-Based Assays and Animal Models

Cell-based assays and animal models for immune diseases are useful in practicing certain embodiments of the invention. Certain cell-based assays provided in the Examples below are useful, e.g., for testing the efficacy of IL-22 antagonists or agonists.

In vivo animal models are also useful in practicing certain embodiments of the invention. Exemplary animal models are also described in the Examples below. The in vivo nature of such models makes them predictive of responses in human patients. Animal models of immune related diseases include both non-recombinant and recombinant (transgenic) animals. Non-recombinant animal models include, for example, rodent, e.g., murine models. Such models can be generated by introducing cells into syngeneic mice using standard techniques, e.g., subcutaneous injection, tail vein injection, spleen implantation, intraperitoneal implantation, implantation under the renal capsule, etc.

Graft-versus-host disease models provide a means of assessing T cell reactivity against MHC antigens and minor transplant antigens. Graft-versus-host disease occurs when immunocompetent cells are transplanted into immunosuppressed or tolerant patients. The donor cells recognize and respond to host antigens. The response can vary from life threatening severe inflammation to mild cases of diarrhea and weight loss. A suitable procedure for assessing graft-versus-host disease is described in detail in Current Protocols in Immunology, above, unit 4.3.

An animal model for skin allograft rejection is a means of testing the ability of T cells to mediate in vivo tissue destruction and a measure of their role in transplant rejection. The most common and accepted models use murine tail-skin grafts. Repeated experiments have shown that skin allograft rejection is mediated by T cells, helper T cells and killer-effector T cells, and not antibodies. Auchincloss, H. Jr. and Sachs, D. H., Fundamental Immunology, 2nd ed., W. E. Paul ed., Raven Press, NY, 1989, 889-992. A suitable procedure is described in detail in Current Protocols in Immunology, above, unit 4.4. Other transplant rejection models which can be used to test the compounds of the invention are the allogeneic heart transplant models described by Tanabe, M. et al, Transplantation (1994) 58:23 and Tinubu, S. A. et al, J. Immunol. (1994) 4330-4338.

Contact hypersensitivity is a simple in vivo assay for cell mediated immune function (delayed type hypersensitivity). In this procedure, cutaneous exposure to exogenous haptens which gives rise to a delayed type hypersensitivity reaction which is measured and quantitated. Contact sensitivity involves an initial sensitizing phase followed by an elicitation phase. The elicitation phase occurs when the T lymphocytes encounter an antigen to which they have had previous contact. Swelling and inflammation occur, making this an excellent model of human allergic contact dermatitis. A suitable procedure is described in detail in Current Protocols in Immunology, Eds. J. E. Cologan, A. M. Kruisbeek, D. H. Margulies, E. M. Shevach and W. Strober, John Wiley & Sons, Inc., 1994, unit 4.2. See also Grabbe, S, and Schwarz, T, Immun. Today 19 (1): 37-44 (1998).

Additionally, the compositions of the invention can be tested on animal models for psoriasis-like diseases. For example, compositions of the invention can be tested in the scid/scid mouse model described by Schon, M. P. et al, Nat. Med. (1997) 3:183, in which the mice demonstrate histopathologic skin lesions resembling psoriasis. Another suitable model is the human skin/scid mouse chimera prepared as described by Nickoloff, B. J. et al, Am. J. Path. (1995) 146:580. Another suitable model is described in Boyman et al., J Exp Med. (2004) 199(5):731-6, in which human prepsoriatic skin is grafted onto AGR129 mice, leading to the development of psoriatic skin lesions.

Knock out animals can be constructed which have a defective or altered gene encoding a polypeptide identified herein, as a result of homologous recombination between the endogenous gene encoding the polypeptide and a DNA molecule in which that gene has been altered. For example, cDNA encoding a particular polypeptide can be used to clone genomic DNA encoding that polypeptide in accordance with established techniques. A portion of the genomic DNA encoding a particular polypeptide can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells. A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the polypeptide.

2. Screening Assays for Drug Candidates

Screening assays for drug candidates are designed to identify compounds that bind to or complex with a polypeptide identified herein or a biologically active fragment thereof, or otherwise interfere with the interaction of a polypeptide with other cellular proteins. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art. All assays are common in that they call for contacting a test compound with a polypeptide identified herein under conditions and for a time sufficient to allow the polypeptide to interact with the test compound.

In binding assays, the interaction is binding and the complex formed can be isolated or detected in the reaction mixture. In a particular embodiment, a polypeptide or the test compound is immobilized on a solid phase, e.g., on a microtiter plate, by covalent or non-covalent attachments. Non-covalent attachment generally is accomplished by coating the solid surface with a solution of the polypeptide or test compound and drying. Alternatively, an immobilized antibody, e.g., a monoclonal antibody specific for a polypeptide to be immobilized, can be used to anchor the polypeptide to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labelled antibody specifically binding the immobilized complex.

If the test compound interacts with but does not bind to a particular polypeptide identified herein, its interaction with that protein can be assayed by methods well known for detecting protein-protein interactions. Such assays include traditional approaches, such as, cross-linking, co-immunoprecipitation, and co-purification through gradients or chromatographic columns. In addition, protein-protein interactions can be monitored by using a yeast-based genetic system described by Fields and co-workers [Fields and Song, Nature (London) 340, 245-246 (1989); Chien et al., Proc. Natl. Acad. Sci. USA 88, 9578-9582 (1991)] as disclosed by Chevray and Nathans, Proc. Natl. Acad. Sci. USA 89, 5789-5793 (1991). Many transcriptional activators, such as yeast GAL4, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

To identify compounds that interfere with the interaction of a polypeptide identified herein and other intra- or extracellular component(s), a reaction mixture may be prepared containing the polypeptide and the component under conditions allowing for the interaction of the polypeptide with the component. To test the ability of a test compound to inhibit the interaction, the reaction mixture is prepared in the absence and in the presence of the test compound. If there is a decrease in the interaction of the polypeptide with the component in the presence of the test compound, then the test compound is said to inhibit the interaction of the polypeptide with the component.

In certain embodiments, methods for identifying agonists or antagonists of an AMP comprise contacting an AMP with a candidate agonist or antagonist molecule and measuring a detectable change in one or more biological activities normally associated with the AMP. Such activities include, but are not limited to, those described in the Examples below.

In one embodiment, the present invention provides methods for identifying agonists of an IL-22 polypeptide comprise contacting an IL-22 polypeptide with a candidate agonist molecule and measuring a detectable change in one or more biological activities normally associated with the IL-22 polypeptide. Such activities include, but are not limited to, those described in the Examples below.

3. Antibody Binding Assays

Antibody binding studies may be carried out in any known assay method, such as competitive binding assays, direct and indirect sandwich assays, and immunoprecipitation assays. Zola, Monoclonal Antibodies. A Manual of Techniques, pp. 147-158 (CRC Press, Inc., 1987).

Competitive binding assays rely on the ability of a labeled standard to compete with the test sample analyte for binding with a limited amount of antibody. The amount of target protein in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

Immunohistochemistry may also be used to determine the cellular location of an antigen to which an antibody binds. For immunohistochemistry, the tissue sample may be fresh or frozen or may be embedded in paraffin and fixed with a preservative such as formalin, for example. Articles of Manufacture

In another embodiment, the present invention provides an article of manufacture comprising compositions useful for the diagnosis or treatment of the microbial disorders described herein. The article of manufacture comprises a container and an instruction. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is usually a polypeptide, an antibody, an agonist, or an antagonist of the invention. An instruction or label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

In one embodiment, the invention provides an article of manufacture, comprising:
(a) a composition of matter comprising an AMP or modulator thereof (e.g., an IL-22 agonist);
(b) a container containing said composition; and
(c) a label affixed to said container, or a package insert included in said container, referring to the use of said agonist in the treatment of an microbial disorder. The composition may comprise an effective amount of the agonist.

### EXAMPLES

The following are examples of methods and compositions of the invention, and are provided herein for illustrative purposes, and are not intended to limit the scope of the present invention. It is understood that various other embodiments may be practiced, given the general description provided herein.

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

The data presented herein demonstrate for the first time that IL-22 is one of the key cytokines that bridges adaptive immune response and innate epithelial defense during early infection of an A/E bacterial pathogen. As shown herein, the induction of RegIIIβ and RegIIIγ also indicates that IL-22 may have broader functions in controlling various bacterial infections. The data further supports the role of Th17 cells and their effector cytokines in infectious diseases and autoimmune diseases. Finally, the present studies indicate that IL-22 and its downstream products, such as RegIIIβ and RegIIIγ, may be beneficial for the treatment of certain infectious diseases.

### Example 1

### IL-23 is Essential for IL-22 Regulation During an Infectious Disease Process

The data herein demonstrate that IL-23 is essential for IL-22 regulation during an infectious disease process.

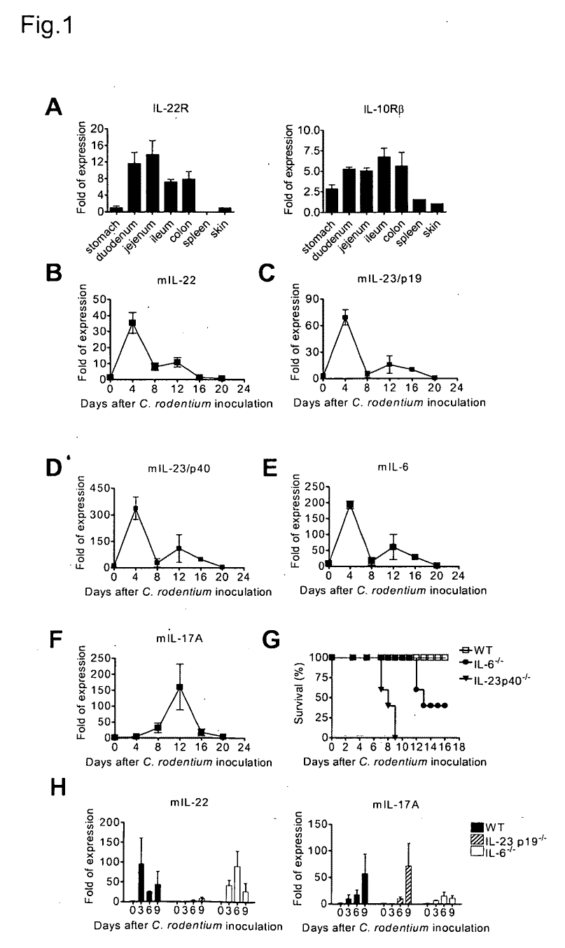

Both IL-22 receptor pairs, IL-22R and IL-10Rβ chains, are expressed in the GI tract of wildtype C57Bl/6 mice (FIG. 1A). Their expression in the duodenum, jejunum, ileum, and colon are higher than they are in the skin, a tissue where IL-22 has been shown to induce hyperplasia. Consistently both colonic epithelial cells and subepithelial myofibroblasts have been reported to respond to IL-22. During C. rodentium infection, IL-22 was induced in the colon of wildtype mice (FIG. 1B), as were cytokines that promote Th17 cell differentiation, including the p19 and p40 subunit of IL-23 (FIG. 1C-D), and IL-6 (FIG. 1E). All of these cytokines were rapidly induced, with peak expression around day 4 post inoculation. In contrast, IL-17 induction had slower kinetics and reached its maximum-level at day 12 post inoculation (FIG. 1F).

Since either IL-23 or IL-6 promotes IL-22 production from T cells in vitro, the present inventors sought to first define their role in regulation of IL-22 production during C. rodentium infection. When comparing the survival rate of wildtype, p19^{-/-}, p40^{-/-}, and IL-6^{-/-} mice after C. rodentium infection, we consistently found all the mice from either the p40^{-/-} group (FIG. 1G) or the p19^{-/-} group (data not shown) died 10 days post inoculation. Interestingly, 60% mortality was also observed in the IL-6^{-/-} group around day 12 (FIG. 1G), indicating that IL-6 is required, to a certain extent, for a total control of C. rodentium infection. Next we examined IL-22 and IL-17 expression in both p19^{-/-} and IL-6^{-/-} mice (FIG. 1H). While, IL-17 expression was not altered in p19^{-/-} mice (15), induction of IL-22 was diminished in p19^{-/-} mice compared to wildtype mice. In IL-6^{-/-} mice, however, while the peak level of IL-22 was comparable to that of wildtype mice, its induction was significantly delayed (FIG. 1H). Furthermore, in IL-6^{-/-} mice, the induction of IL-17 was significantly reduced, consistent with an essential role of IL-6 for IL-17 production.

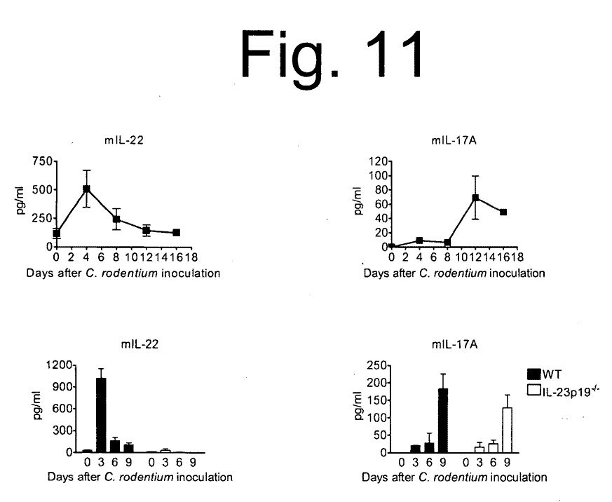

Directly measuring IL-22/IL-17 proteins by ELISA in ex vivo culture of colons from infected mice confirmed the kinetics of IL-22/IL-17 production and the absence of IL-22 induction from p19^{-/-} mice during C. rodentium infection (FIG. 11). These data for the first time demonstrate that IL-23 is essential for IL-22 regulation during an infectious disease process.

These data for the first time demonstrate that IL-23 is essential for IL-22 regulation during an infectious disease process.

### Example 2

### IL-22 is a Key Downstream Effector Cytokine that Contributes to the Biology of IL-23 in Controlling Microbial Infection

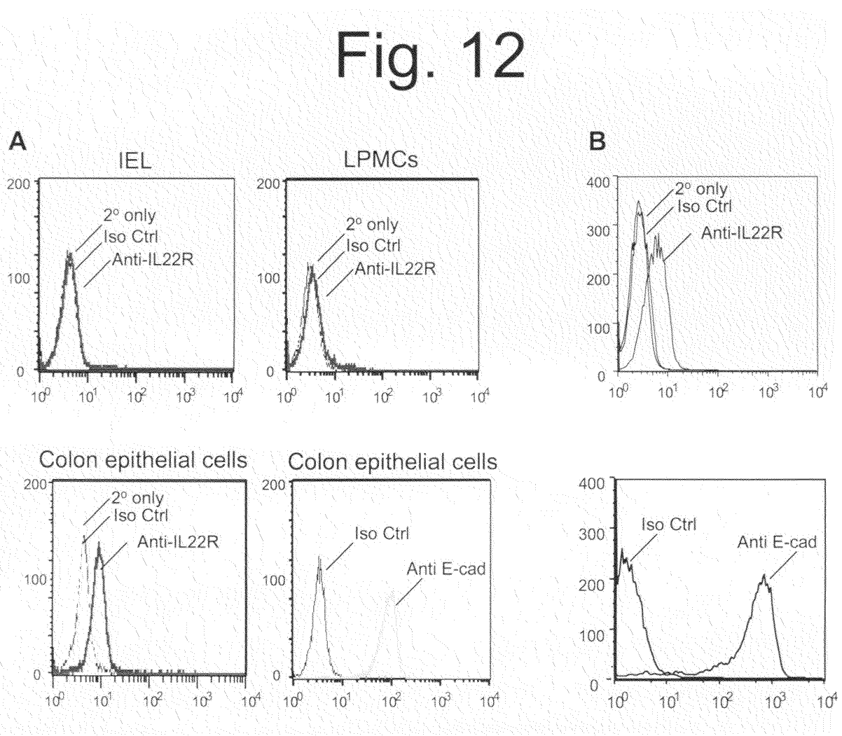

The altered regulation of IL-22 in both IL-23 deficient and IL-6^{-/-} mice indicated that IL-22 may play a critical role in the host defense against C. rodentium infection. To further examine the role of IL-22, IL-22^{-/-} mice were inoculated with C. rodentium. While wildtype littermates transiently lost weight but were able to fully recover after day 6, IL-22^{-/-} mice continued losing weight following C. rodentium infection (FIG. 2A). About 80% of IL-22^{-/-} mice became moribund or died 12 days post C. rodentium inoculation (FIG. 2A). Histologic analysis of the colons from day 8 infected IL-22^{-/-}mice demonstrated increased mucosal thickness when compared with that of WT mice (FIG. 2B). Coincidentally, there was also increased submucosal inflammation (Arrow, FIG. 2B). Furthermore, while in control mice, C. rodentium infection was predominantly superficial, large numbers of bacteria penetrated deeply into colonic crypts in IL-22^{-/-} mice (Arrows, FIG. 2C). FACS analysis with an anti-IL-22R antibody (FIG. 12) revealed that IL-22R was expressed by E-cadherin positive primary murine colonic epithelial cells, but not by CD45⁺ intra-epithelial lymphocytes (IEL) or lamina propria mononuclear cells (LPMCs) (FIG. 12A). Similarly, primary human colonic epithelial cells also expressed IL-22R (FIG. 12B). These data suggest that colonic epithelial cells were directly targeted by IL-22.

These data support the importance of IL-22 in host defense against C. rodentium infection, and indicate that IL-22 may be one of the key downstream effector cytokines that contribute to the biology of IL-23 in controlling microbial infections.

### Example 3

### IL-17A and IL-17F Pathways are not Required for Host Defense Against C. rodentium Infection

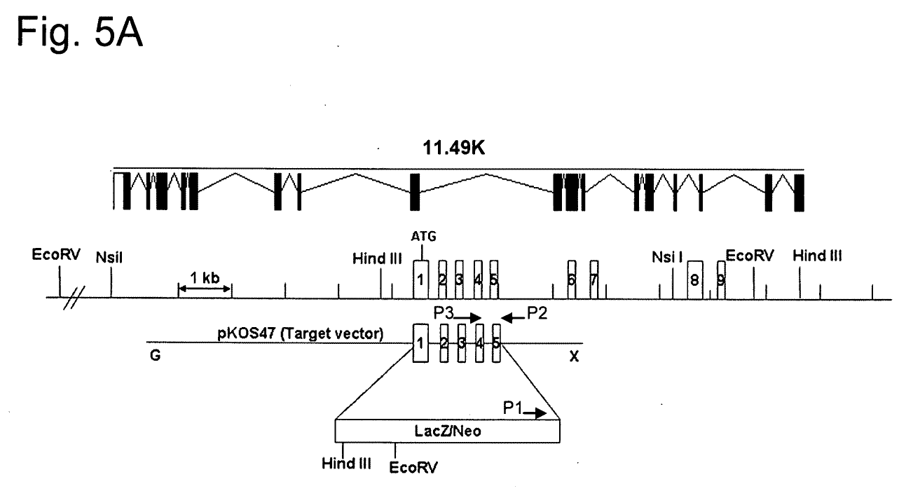

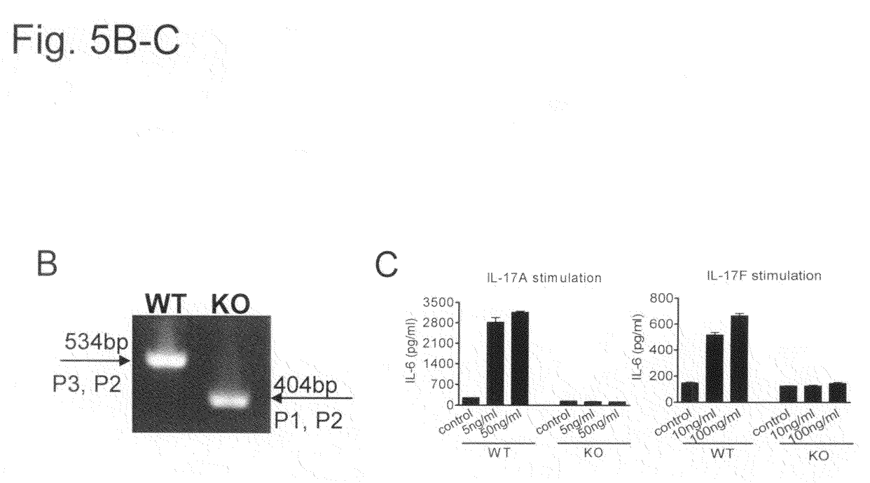

The partial impairment of host defense in IL-6^{-/-} mice against C. rodentium could also be explained by the delayed induction of IL-22 in these mice (FIG. 1H, left panel). However, it is also possible that lethality in C. rodentium infected IL-6^{-/-} mice may have been due to their inability to upregulate IL-17 (FIG. 1H, right panel). The IL-17 pathway is crucial for the control of many extracellular bacterial infections, such as Klebsiella pneumoniae. IL-17 signals through IL-17R and IL-17RC (D. Toy et al., J Immunol 177, 36 (Jul. 1, 2006)), and induces proinflammatory responses from many cell types, including epithelial cells (J. Witowski, K. Ksiazek, A. Jorres, Cell Mol Life Sci 61, 567 (March, 2004)). To analyze the role of the IL-17 pathway during C. rodentium infection, IL-17RC^{-/-} mice were generated (FIG. 5). Compared to wildtype littermates, there was no obvious defect in IL-17RC^{-/-} mice in terms of development or composition of T cells, B cells and other immune cells (data not shown). However, fibroblasts generated from the tail tip (FIG. 5C) or lung tissue (data not shown) of IL-17RC^{-/-} mice were completely incapable of producing IL-6 when stimulated with either IL-17A or IL-17F, indicating that IL-17RC is an essential receptor for both IL-17A and IL-17F mediated functions. Following C. rodentium inoculation, both IL-17RC^{-/-} mice and wildtype littermates survived the course of infection without any significant loss of weight (FIG. 2D) or any histologic differences in the colon (data not shown).

These results indicate that IL-17A and IL-17F pathways were not required for host defense against C. rodentium infection, directly excluding the possibility that defective IL-17 production is the major cause of observed mortality in IL-6^{-/-} mice. Thus, the delayed induction of IL-22 observed in IL-6−/− mice might be the reason that these mice were incapable of surviving the infection. Other factors downstream of IL-6, however, may also be important. The results from IL-6^{-/-} mice imply that the early induction of IL-22 might be critical for the host to mount a sufficient response against C. rodentium infection in order to prevent lethality.

### Example 3

### IL-22 Plays a Critical Role in the Early Stage of Bacterial Infection

To determine whether early induction of IL-22 is critical for the host to mount a sufficient response against C. rodentium infection in order to prevent lethality, anti-IL-22 neutralizing antibody was administrated every other day starting either at day 0 or at day 8 post inoculation of C. rodentium. As expected, mice that received anti-IL-22 mAb at the same time as the bacterial inoculation continued to lose weight, and all became moribund or died 12 days post inoculation. In contrast, all isotype control antibody treated animals survived (FIG. 2E). Mice that received anti-IL-22 mAb starting 8 days post inoculation had a similar outcome as did isotype mAb treated mice, with full recovery from infection.

Therefore, these data indicate that IL-22 plays a critical role in the early stage of C. rodentium infection, but plays no role during the later phase of host defense when bacteria are being eradicated.

### Example 4

### IL-19, IL-20, and IL-24 are Dispensable for Host Defense Against Bacterial Infection

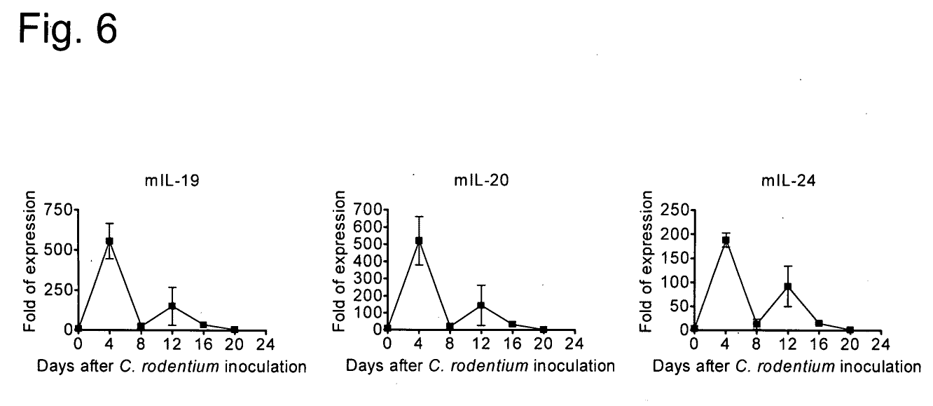

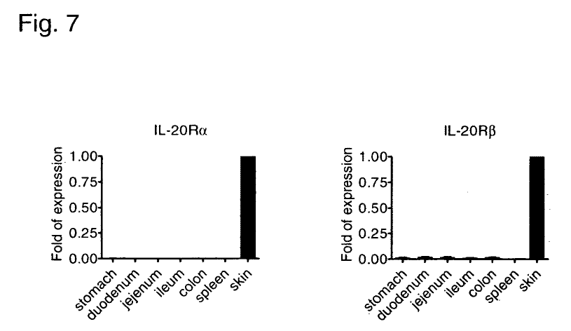

Other IL-10 family cytokines, IL-19, IL-20, and IL-24, all induce similar biological functions as those induced by IL-22 in human epidermal keratinocytes (S. M. Sa et al., J Immunol 178, 2229 (Feb. 15, 2007).). IL-19, IL-20, and IL-24 were all upregulated in wildtype mouse colon during C. rodentium infection (FIG. 6). They may, therefore, play similar role as does IL-22 during C. rodentium infection. IL-19 signals through IL-20Rα and IL-20Rβ chains. IL-20 and IL-24 can signal through two different receptor pairs, IL-20Rα/IL-20Rβ and IL-22R/IL-20Rβ (J.-C. Renauld, Nature Reviews Immunology 3, 667 (2003)). Therefore, IL-20Rβ is the common receptor chain for these three cytokines. In the GI tract, expression of IL-20Rα and IL-20Rβ chains was significantly lower than the expression of these chains in skin (FIG. 7).

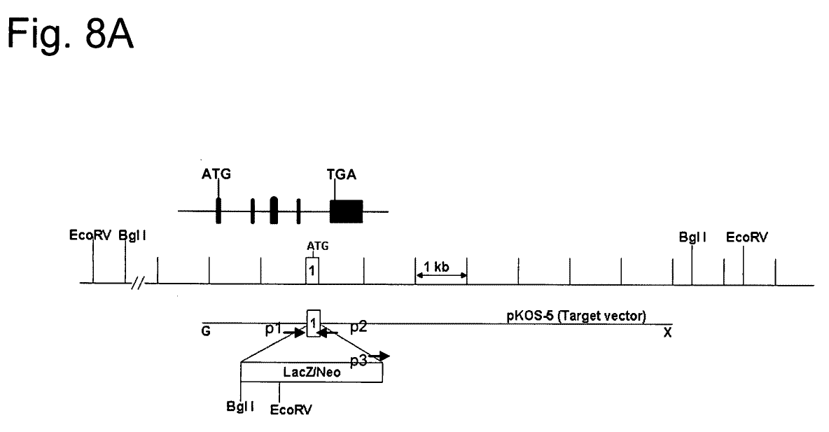

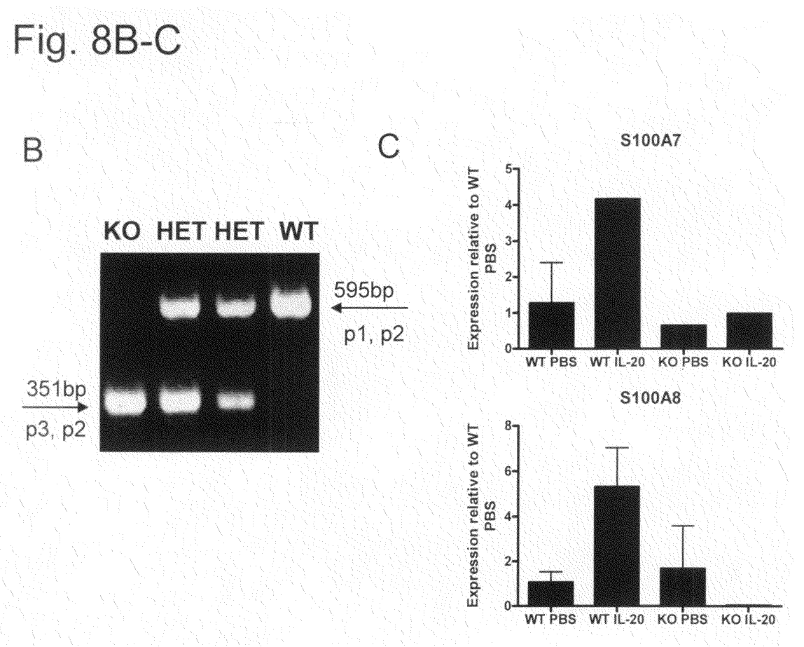

To critically address the role of these three cytokines during C. rodentium infection, IL-20Rβ^{-/-} mice were generated (FIG. 8). These mice exhibited normal development with similar lymphocyte composition and development in all major lymphoid organs when compared to wildtype mice (data not shown). The ear skin from these mice failed to upregulate S100 family proteins when treated with recombinant IL-20, indicating a defect in IL-20 signaling in vivo (FIG. 8C).

IL-20Rβ^{-/-} mice survived C. rodentium infection as well as wildtype mice did (FIG. 2F), demonstrating that IL-19, IL-20, and IL-24 are dispensable for host defense against C. rodentium.

### Example 5

### IL-22 Deficiency May Compromise Epithelial Integrity During the Early Stage of C. rodentium Infection

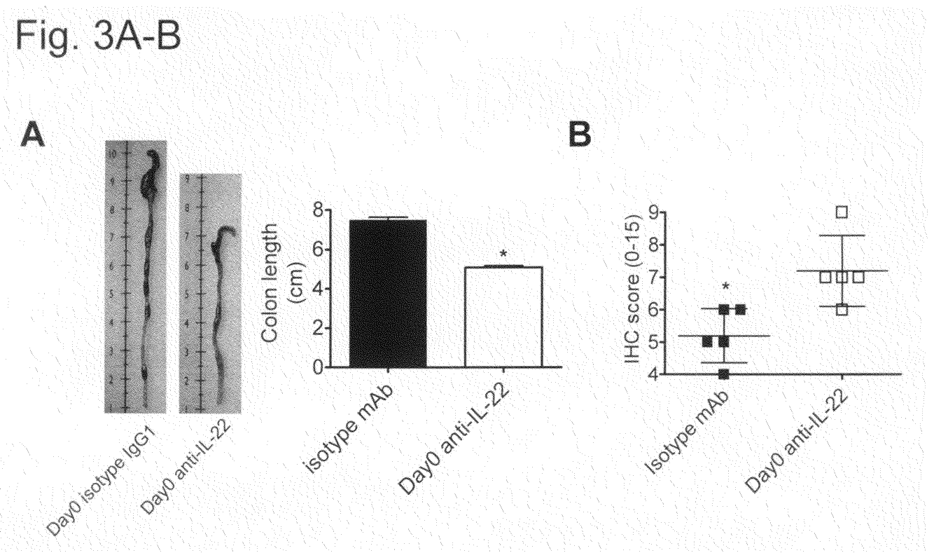

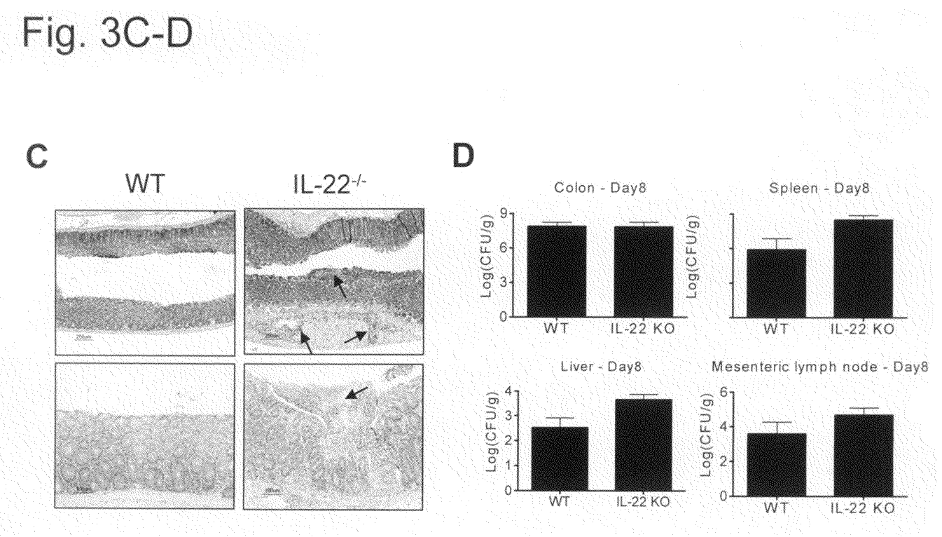

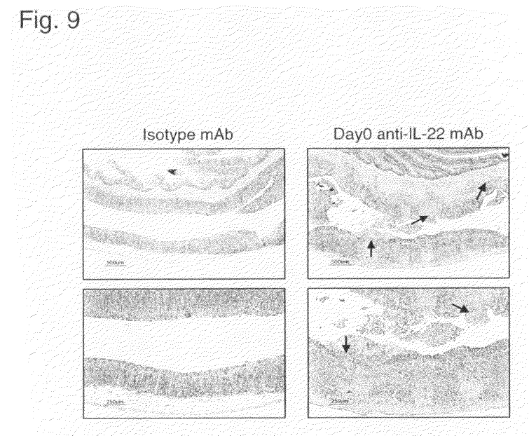

The present inventors examined the downstream mechanisms of IL-22 during C. rodentium infection. Both IL-22^{-/-} mice and wildtype mice treated with anti-IL-22 mAb on day 0 developed more severe bloody diarrhea and an increased incidence of rectal prolapse compared to control mice 8 days post inoculation of C. rodentium (data not shown). Colons from IL-22^{-/-} mice (data not shown) or day 0 anti-IL-22 mAb treated mice were thickened and shortened 10 days post inoculation (FIG. 3A), as well as having a smaller cecum, compared to control mice. Histologic analysis further revealed increased inflammation in colons lacking IL-22 signaling (FIG. 3B). There were also marked multifocal mucosal ulceration and multiple foci of transmural inflammation in both IL-22^{-/-} and anti-IL-22 mAb treated mice (FIG. 3C, and FIG. 9). Furthermore, the bacterial burdens in mesenteric lymph node, spleen, and liver of IL-22^{-/-} mice were significantly increased compared to those of wildtype mice. Interestingly, the difference in bacterial burdens in colons of wildtype mice and IL-22^{-/-} mice was negligible (FIG. 3D). Consistent with these results, there was also evidence of systemic bacterial spread, particularly in the livers of IL-22^{-/-} mice, where multifocal hepatocellular necrosis with embolic microabscessation was evident (FIG. 3E).

In conclusion, these data indicate that the epithelial integrity is compromised in IL-22^{-/-} mice during the early stage of C. rodentium infection.

### Example 6

### IL-22 Deficiency Leads to a Reduction in Anti-Bacterial IgG Titers

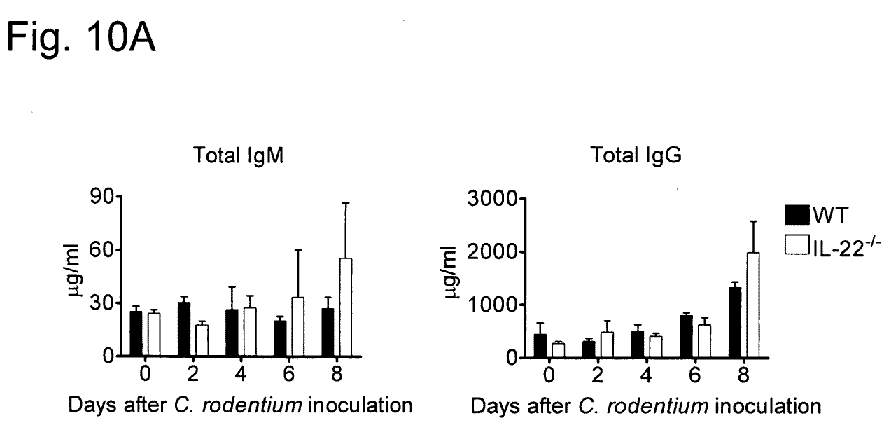

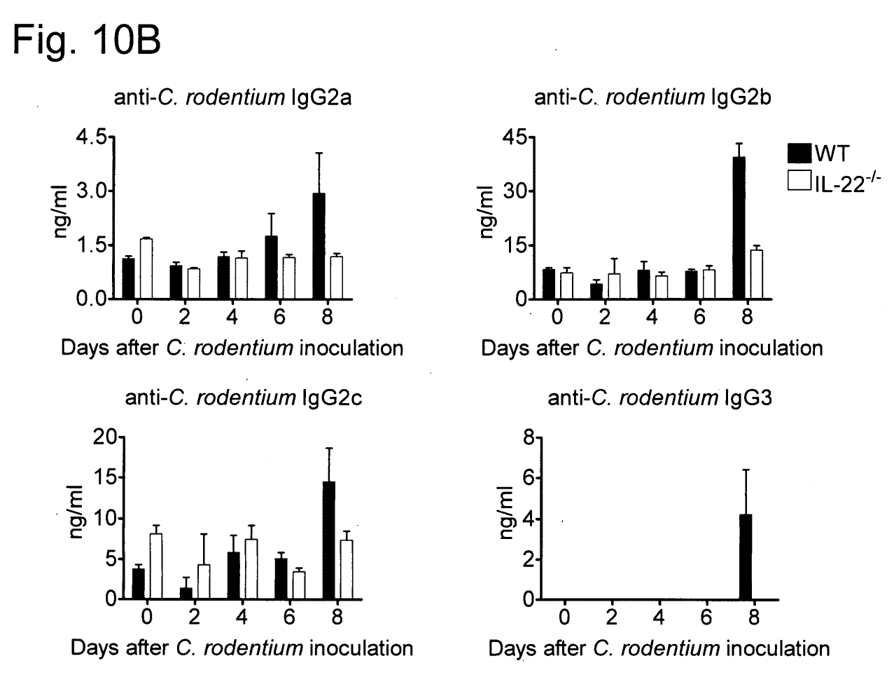

Previous studies established the essential role of anti-C. rodentium antibodies in the clearance of bacteria. Transferring serum from wildtype mice post-infection fully rescued CD4^{-/-} mice from death following C. rodentium challenge (10). In our studies, IL-22 deficient mice became moribund or died starting around day 8, when antibody responses were not fully developed in wildtype mice (FIG. 3F). On day 8, anti-C. rodentium antibody titers were 50 fold less than those on day 16 in wildtype mice. However, when we compared the titers of anti-C. rodentium antibodies on day 8 from wildtype and IL-22^{-/-} mice, there was an unexpected significant reduction in the anti-C. rodentium IgG titer in IL-22^{-/-} mice compared to that in wildtype mice (FIG. 3G). In contrast, there was no decrease in total IgG, IgM, IgA or anti-C. rodentium IgM and IgA titers in IL-22−/− mice (FIG. 10A and data not shown). Further IgG subtype analysis revealed that while there was no anti-C. rodentium IgG1 in either wildtype or IL-22^{-/-} mice 8 days post inoculation, other anti-C. rodentium IgG subtypes, including IgG2a, IgG2b, IgG2c and IgG3, were all significantly reduced in IL-22^{-/-} mice (FIG. 10B). It was unlikely that differences in anti-bacterial specific IgG contributed to clearance of C. rodentium from the colon at this time point, especially since IgG is not targeted to the colonic mucosal lumen, and colonic bacterial burdens in both wildtype and IL-22^{-/-} mice were similar (FIG. 3D). It is possible, though that circulating anti-C. rodentium IgG may be important in controlling penetration of C. rodentium through the intestinal epithelial barrier, and preventing systemic spread, since a recent study demonstrated that circulating IgG, but not secretory IgA or IgM, was required for systemic clearance of C. rodentium (C. Maaser et al., Infect. Immun. 72, 3315 (Jun. 1, 2004)). How IL-22 deficiency leads to a reduction in anti-bacterial IgG titers is unclear. It is unlikely that IL-22 directly acts on B cells, since the expression of IL-22R is not detectable on B cells (S. Lecart et al., Int. Immunol. 14, 1351 (Nov. 1, 2002)). Nonetheless, reduced anti-C. rodentium IgG might be one of the factors that contribute to the defective host defense response in IL-22^{-/-} mice during C. rodentium infection.

### Example 7

### IL-22 was Indispensable for the Induction of Anti-Microbial Lectins, such as RegIIIβ and RegIIIγ, from Colonic Epithelial Cells During Bacterial Infection

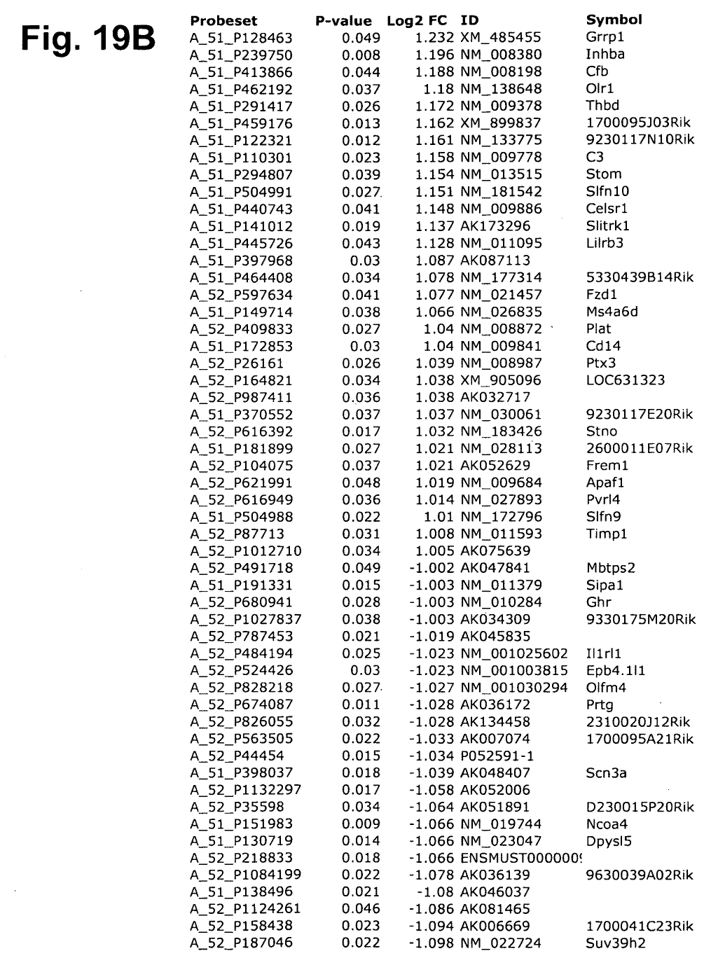

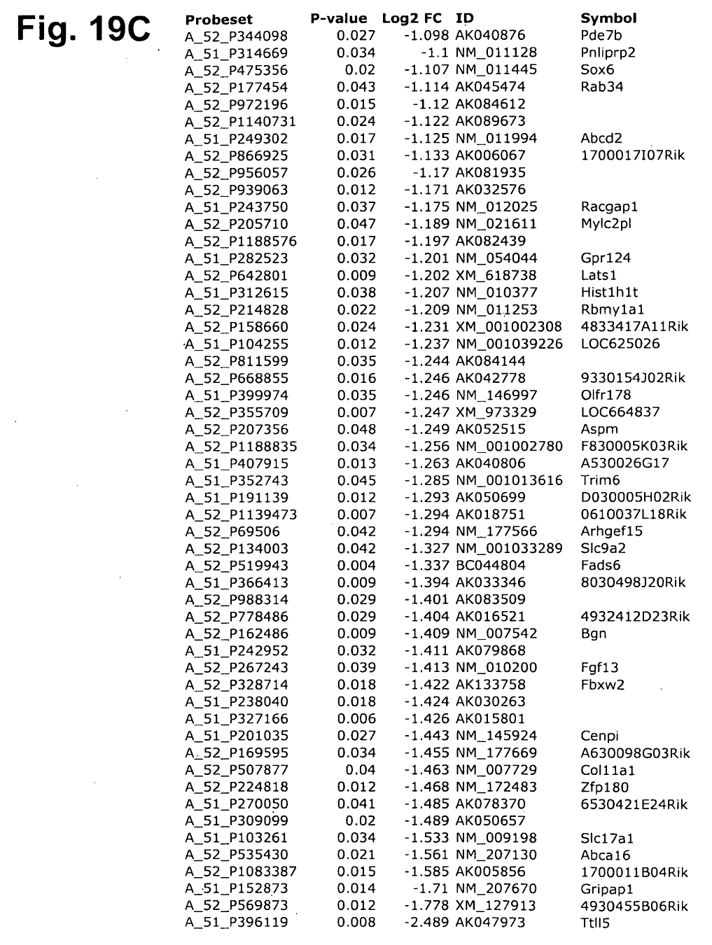

IL-22 treatment of colon tissues from uninfected wildtype mice ex vivo upregulated many anti-microbial proteins, including S100A8, S100A9, RegIIIβ, RegIIIγ, haptoglobin, SAA3, and lactotransferrin by microarray analysis (FIGS. 4A, 19, and 20). The induction of these proteins was confirmed by real-time RT-PCR (FIG. 4B and data not shown). During C. rodentium infection, however, only S100A8, S100A9, RegIIIβ and RegIIIγ were differentially expressed in IL-22^{-/-} mice compared to wildtype mice (FIG. 4C). All other genes were either not induced or were not different in colons of wildtype vs. IL-22^{-/-} mice (data not shown). Expression of both S100A8 and S100A9 was slightly higher in the colons of IL-22^{-/-} mice than it was in wildtype colon on day 4 and day 6, suggesting that differential expression of these proteins was most likely not responsible for the increased mortality observed in IL-22^{-/-} mice during C. rodentium infection. Differences were not found in the expression of defensins, proteins that are important in host defense of infected epithelium (T. Ganz, Science 286, 420 (Oct. 15, 1999)), between wildtype and IL-22^{-/-} mice (data not shown). Interestingly, the upregulation of RegIIIβ and RegIIIγ observed in wild type mice was completely abolished in IL-22−/− mice post C. rodentium inoculation (FIG. 4C), indicating that these two proteins had potential functions in controlling C. rodentium infection. Both RegIIIβ and RegIIIγ belong to a family of secreted C-type lectin proteins (H. L. Cash, C. V. Whitham, L. V. Hooper, Protein Expression and Purification 48, 151 (2006)). RegIIIβ and RegIIIγ expression levels increase dramatically in response to bacterial colonization as well as following other inflammatory stimuli in mice (S. A. Keilbaugh et al., Gut 54, 623 (May 1, 2005) H. Ogawa et al., Inflammatory Bowel Diseases 9, 162 (2003) H. Ogawa, K. Fukushima, I. Sasaki, S. Matsuno, Am J Physiol Gastrointest Liver Physiol 279, G492 (September, 2000)).

RegIIIβ or RegIIIγ may prevent the invasion of C. rodentium deep into the colonic crypts, as we saw no differences in bacterial burdens from the colons of IL-22^{-/-} vs. wildtype mice (FIG. 3D). Alternatively, RegIIIβ or RegIIIγ proteins may act as autocrine growth factors that play a role in epithelial repair and/or protection in the setting of intestinal inflammation (H. Ogawa et al., Inflammatory Bowel Diseases 9, 162 (2003), S. L. Pull, J. M. Doherty, J. C. Mills, J. I. Gordon, T. S. Stappenbeck, PNAS 102, 99 (Jan. 4, 2005); V. Moucadel et al., Eur J Cell Biol 80, 156 (February, 2001)).

### Example 8

### Adaptive Immunity is not Essential for IL-22 Mediated Early Host Defense Against C. rodentium Infection

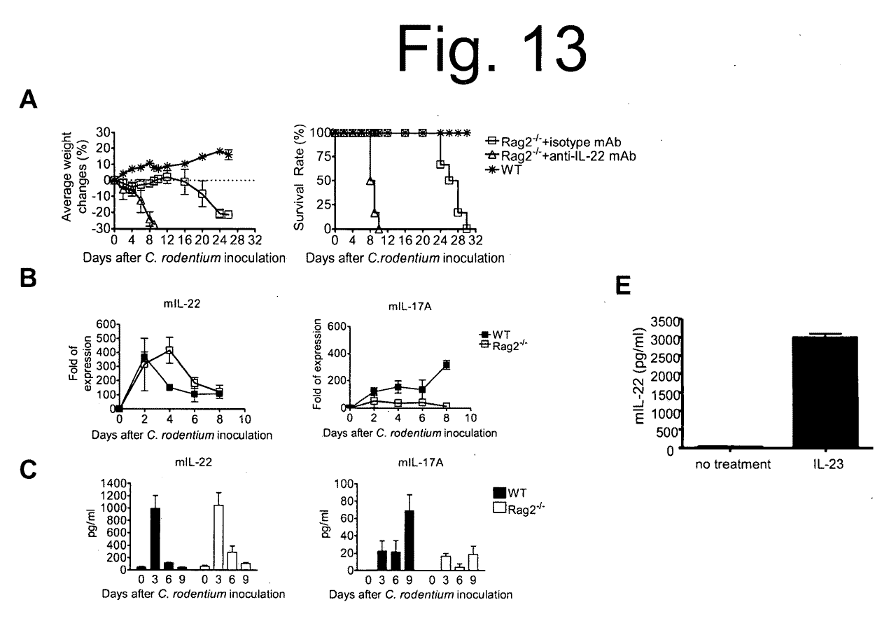

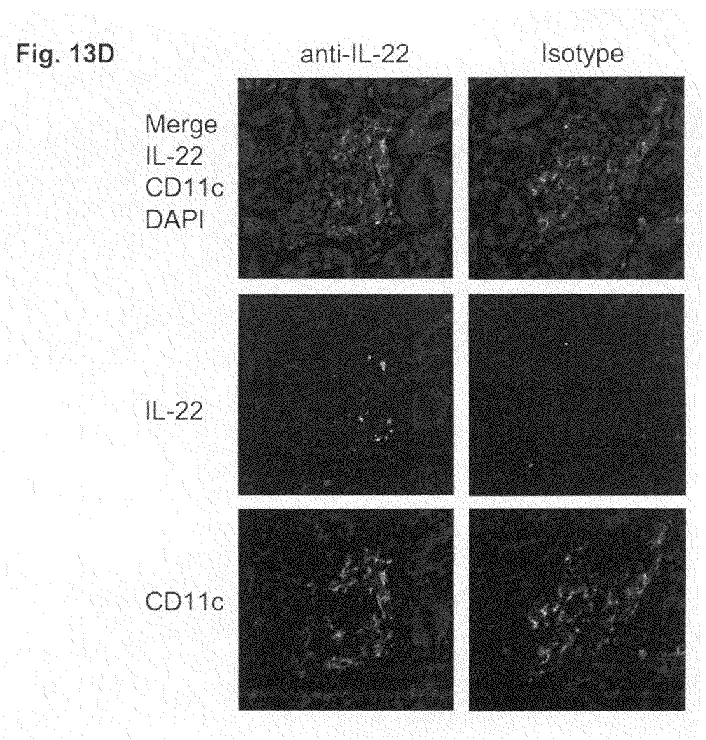

The above data suggested roles of IL-22 in both innate immunity and adaptive immunity. Therefore, we used recombination activating gene 2 deficient (Rag2^{-/-}) mice to critically examine the function of IL-22 in innate vs. adaptive immunity during C. rodentium infection. Rag2^{-/-} mice gradually lost weight and eventually became moribund or died around day 30, due to their lack of B and T cells, and their consequent inability to mount anti-C. rodentium antibody responses (FIG. 13A). In contrast to p19^{-/-} or IL-22^{-/-} mice, none of the Rag2^{-/-} mice lost more than 10% of their body weight or died during the first two weeks of infection. Furthermore, Rag2^{-/-} mice treated with anti-IL-22 mAb lost weight very rapidly (FIG. 13A), similar to WT mice treated with anti-IL-22 mAb (FIG. 2E). All Rag2^{-/-} mice treated with anti-IL-22 mAb became moribund or died around day 10 (FIG. 13). These data suggest that the IL-22 pathway is still active in Rag2^{-/-} mice, and that IL-22 is essential to protect mice from death during the early phase of C. rodentium infection in the absence of adaptive immunity. These data also indicate that reduction in anti-C. rodentium IgG titers was insufficient cause for the morbidity and mortality observed in IL-22^{-/-} mice following C. rodentium infection, as lack of antibody production in Rag2^{-/-} mice alone did not cause rapid weight loss and early death following infection.

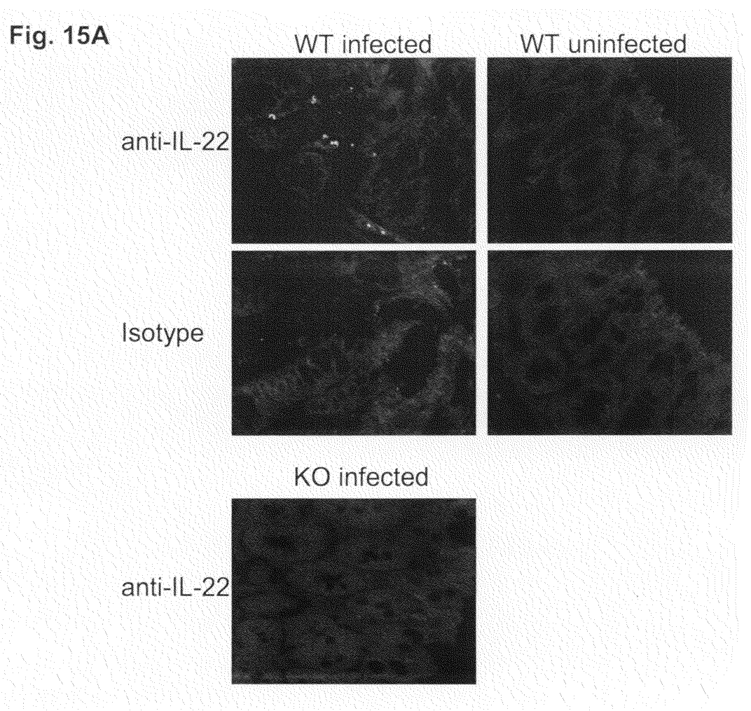

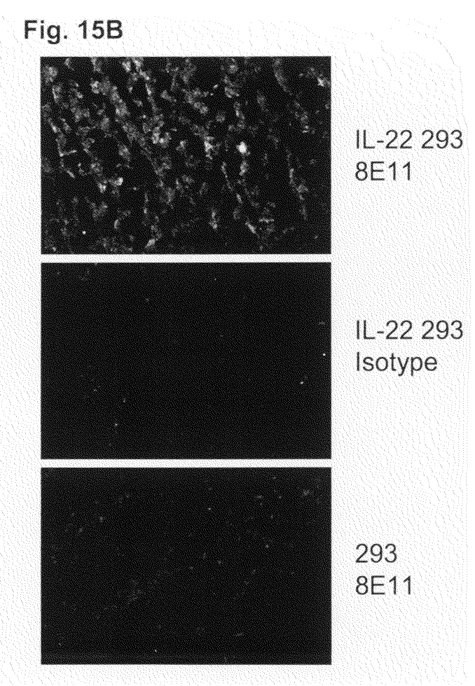

IL-22 production in Rag2^{-/-} mice was comparable with that of WT mice following C. rodentium infection (FIG. 13B). In contrast, induction of IL-17A was significantly reduced in Rag2^{-/-} mice (FIGS. 13B and C). T cells and B cells, therefore, were not the sources of IL-22 in this model. Immunohistochemical staining with an anti-IL-22 mAb (FIG. 15) detected IL-22 positive cells in the colon of WT mice infected with C. rodentium, but not in uninfected colon or colon from infected IL-22^{-/-} mice. IL-22 positive cells primarily co-localized with CD11c+ cell clusters in the colon of Rag2^{-/-} mice (FIG. 13D), but not with F4/80, Gr-1, or DX5 positive cells (data not shown). In addition, IL-23 induced IL-22 production directly from CD11c⁺ DCs in vitro (FIG. 13E). Taken together, our data demonstrate that DCs are one of the major sources of IL-22 production during C. rodentium infection, and that IL-23 can directly promote IL-22 production from DCs.

### Example 9

### RegIII Plays an Important Role During Bacterial Infection

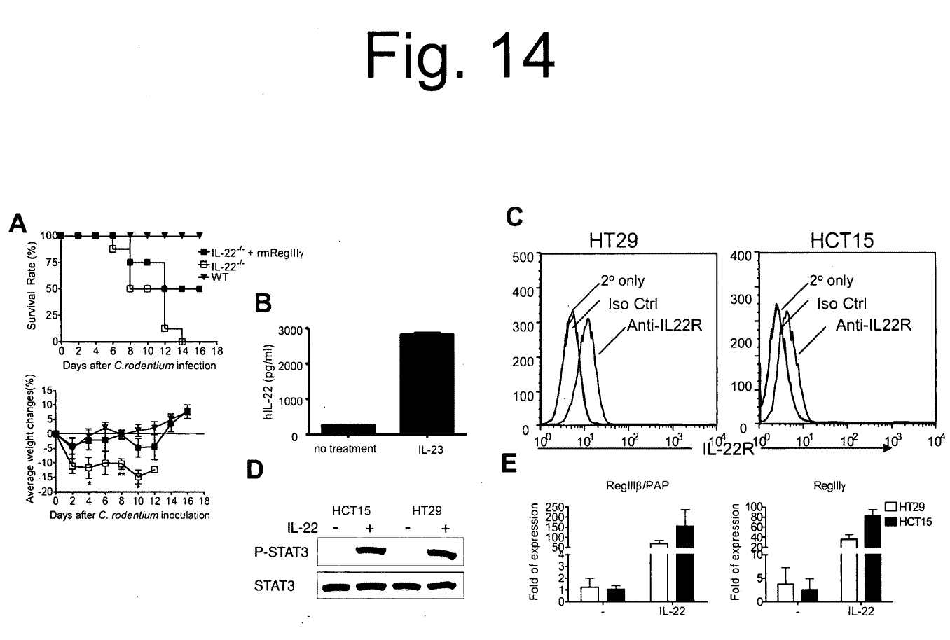

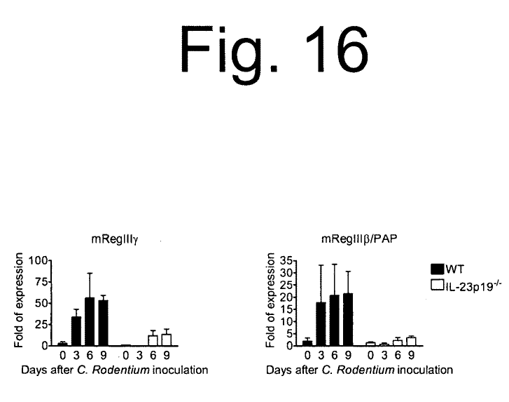

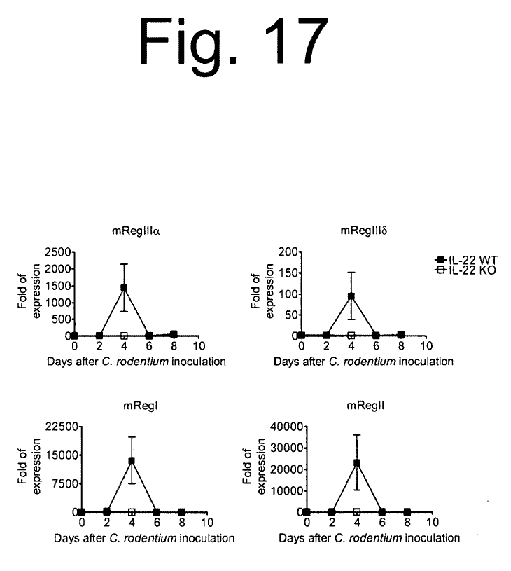

Interestingly, the upregulation of RegIIIβ and RegIIIγ observed in wild type mice was completely abolished in IL-22^{-/-} mice (FIG. 4C), as well as in p19^{-/-} mice, (FIG. 16) post C. rodentium inoculation. RegIIIβ and RegIIIγ belong to a family of secreted C-type lectin proteins. We found that other family members, including RegI, RegII, RegIIIα, and RegIIIδ (FIG. 17), but not RegIV (data not shown), were also upregulated in C. rodentium infected colons, and that their induction was completely abolished in IL-22^{-/-} mice. Exogenous mouse RegIIIγ fusion protein (rmRegIIIγ) significantly protected IL-22^{-/-} mice from the weight loss induced by the C. rodentium infection, and approximately 50% of rmRegIIIγ fusion protein treated animals survived the infection, whereas 100% of control treated IL-22^{-/-} mice became moribund or died (FIG. 14A). These data support the hypothesis that Reg family proteins, such as RegIIIγ, mediate essential functions in controlling C. rodentium infection downstream of IL-22.

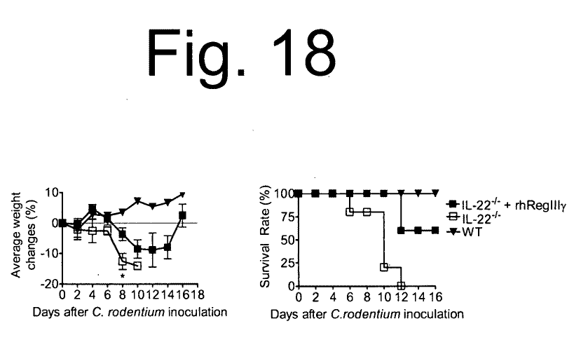

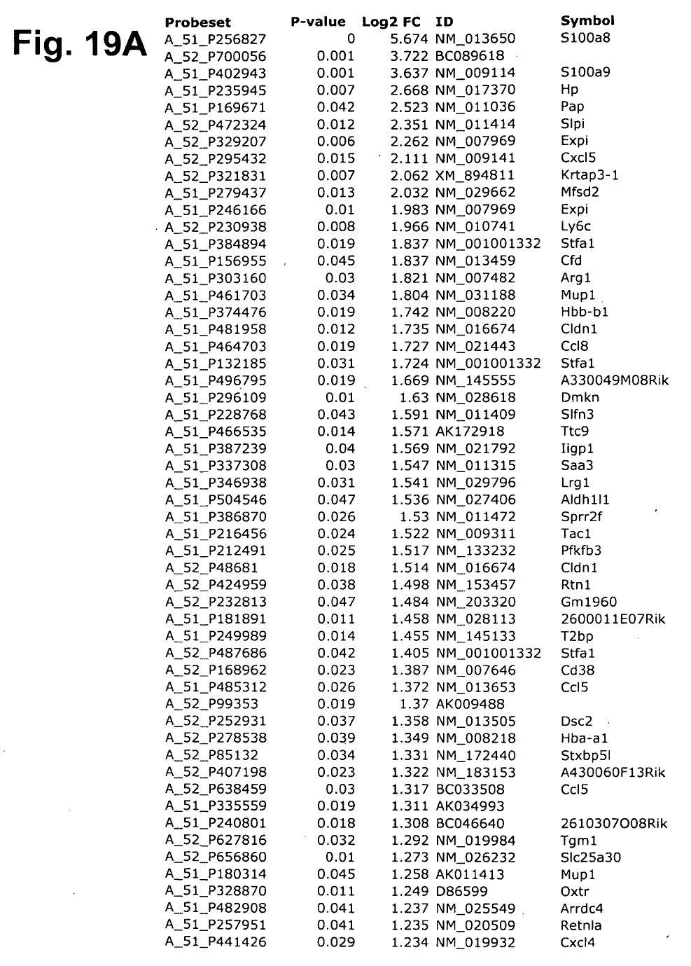

Finally, the presence of the IL-23/IL-22/Reg axis was also validated in a human system. Human IL-23 induces hIL-22 production from human DCs (FIG. 14B). Primary human colonic epithelial cells (FIG. 12B) and the human colonic epithelial cell lines, HT29 and HCT15, express IL-22R (FIG. 14C). In vitro, primary human colonic epithelial cells grew slowly, and gradually lost their expression of IL-22R during expansion (data not shown). Therefore, we used colonic epithelial cell lines to test their response to human IL-22. IL-22 induced STAT3 activation in these colonic epithelial cell lines (FIG. 14D), and both RegIIIβ and RegIIIγ were significantly induced by IL-22 (FIG. 14E). Importantly, human RegIIIγ fusion protein (rhRegIIIγ), like nmRegIIIγ fusion protein, also reduced the mortality of IL-22^{-/-} mice, to 40% following C. rodentium infection, versus 100% mortality in control treated IL-22^{-/-} mice (FIG. 18). In conclusion, our data imply that the IL-22 pathway may play an essential role in controlling bacterial infections, particularly A/E bacterial infections, in the human GI tract.

### SUMMARY

The present inventors demonstrate herein that IL-22 plays an indispensable role in early host defense against attaching and effacing (A/E) bacterial pathogens.

The data herein indicate that IL-22 protects the integrity of the intestinal epithelial barrier and prevents bacterial invasion with systemic spread through two mechanisms. First, IL-22 is involved in the elicitation of the early anti-bacterial IgG responses. Second, IL-22 is indispensable for the induction of anti-microbial lectins, such as RegIIIβ and RegIIIγ, from colonic epithelial cells during bacterial infection. The lack of either or both of these mechanisms may contribute to the compromised host defense response with increased systemic spread and mortality in IL-22^{-/-} mice during C. rodentium infection.

While adaptive immune responses are essential for clearance of these pathogens (L. Bry, M. B. Brenner, J Immunol 172, 433 (Jan. 1, 2004)), cytokines such as IL-22 that are produced by immune cells during the early stages of infection are also necessary for intestinal epithelial cells to elicit a full anti-microbial response and wound healing response in order to prevent systemic invasion of pathogenic bacteria into the host. As shown herein, the induction of RegIIIβ and RegIIIγ also indicates that IL-22 may have broader functions in controlling various bacterial infections. The data further supports the role of Th17 cells and their effector cytokines in infectious diseases and autoimmune diseases. Finally, the present studies indicate that IL-22 and its downstream products, such as RegIIIβ and RegIIIγ, may be beneficial for the treatment of certain infectious diseases.

Materials and Methods

Mice

C57Bl/6, IL-12p40^{-/-}, and IL-6^{-/-} mice were purchased from the Jackson Laboratory. IL-22^{-/-} mice and IL-12p19^{-/-} were generated as described before (11, FIG. 5). IL-17RC^{-/-} and IL-20Rβ^{-/-} mice were generated by Lexicon Pharmaceuticals (The Woodlands, Tex.) by using strategies as described (FIG. 5 and FIG. 8). Briefly, knockout mice were made by standard homologous recombination using depicted targeting vectors. Targeting vectors are electroporated into 129 strain ES cells and targeted clones are identified. Targeted clones are microinjected into host blastocysts to produce chimeras. Chimeras are bred with C57Bl/6 animals to produce F1 heterozygotes. Heterozygotes are intercrossed to produce F2 wild type, heterozygote and homozygote cohorts. Mice used in these studies were genotyped by tail DNA via PCR using a pool of three primers. The primers used for wild-type allele amplification of IL-20Rβ^{-/-} mice were 5′-GTG GAA GCT ACT TGA TGA GTA GGG-3′ (p1) and 5′-AGA TGC GAA AAT GGA GAT TAA AAG-3′ (p2), which yielded a 595 bp product. The primers used for mutant allele amplification of IL-20Rβ^{-/-} mice were 5′-CTA CCC GTG ATA TTG CTG AAG AG-3′ (p3) and p2, which yielded a 351 bp product. The primers used for wild-type allele amplification of IL-17RC^{-/-} mice were 5′-GAG CCT GAA GAA GCT GGA AA-3′ (P3) and 5′-CAA GTG TTG GCA GAG ATG GA-3′ (P2), which yielded a 534 bp product. The primers used for mutant allele amplification of IL-17RC^{-/-} mice were 5′-TCG CCT TCT TGA CGA GTT CT-3′ (P1) and P2, which yielded a 404 bp product.

Bacteria Strain and Infection of Mice

6-8 weeks old mice were fasted for 8 h before oral inoculation with 2×10⁹ C. rodentium strain DBS100 (ATCC 51459; American Type Culture Collection) in a total volume of 200 μl per mouse. While fasting, animals had access to water. Inoculation and all subsequent manipulations were conducted in BL-2 biosafety cabinets. Animals were allowed access to food after inoculation. Bacteria were prepared by incubation with shaking at 37° C. overnight in LB broth. The relative concentration of bacteria was assessed by measuring absorbance at OD600 and each inoculation culture was serially diluted and plated to confirm CFU administered.

Tissue Collection, Histology and CFU Counts

Control or infected mice were inoculated as described. Samples of whole blood, spleen, liver, mesenteric lymph node, and colon were removed under aseptic conditions. The colon was dissected to the anal canal, and the terminal 0.5-cm piece was used for CFU analysis. Proximal segments were fixed in 10% neutral buffered formalin. Sections were stained with H&E to evaluate tissue pathology. Spleen, liver, mesenteric lymph node, and colon were weighed and homogenized. Homogenates were serially diluted and plated in triplicates to MacConkey agar (Remel). C. rodentium colonies were identified as pink colonies. Colonies were counted after 24 h of incubation at 37° C. to determine the log₁₀ CFU per gram of tissues.

RNA Isolation and Real-Time RT-PCR

Cell and tissue RNA were isolated by RNeasy Mini Kit (Qiagen) according to the manufacture's directions. Real-time RT-PCR was conducted on an ABI 7500 Real-Time PCR system (Applied Biosystems) with primers and probes using TaqMan one-step RT-PCR master mix reagents (Applied Biosystems). The sequences for primers and probes were as follows: mIL-22, forward, 5′-TCC GAG GAG TCA GTG CTA AA-3′, reverse, 5′-AGA ACG TCT TCC AGG GTG AA-3′, and probe, 5′-TGA GCA CCT GCT TCA TCA GGT AGC A-3′ (FAM, TAMRA); mIL-17A, forward, 5′-GCT CCA GAA GGC CCT CAG A-3′, reverse, 5′-CTI TCC CTC CGC ATT GAC A-3′, and probe, 5′-ACC TCA ACC GTT CCA CGT CAC-3′ (FAM, TAMRA); mouse ribosomal housekeeping gene RPL-19, forward, 5′-GCA TCC TCA TGG AGC ACA T-3′, reverse, 5′-CTG GTC AGC CAG GAG CTT-3′, and probe, 5′-CTT GCG GGC CTT GTC TGC CTT-3′ (FAM, TAMRA); mIL-19, forward, 5′-AGC CTG GAT TGA CAG GAA TC-3′, reverse, 5′-GAT AAT CAG ACG AGG CGT TTC-3′, and probe, 5′-TCT GGA AAC TCC TGC AGC CTG ACA C-3′ (FAM, TAMRA); mIL-20, forward, 5′-TTT GGG AGA ACT AGG CAT TCT T-3′, reverse, 5′-TCT TGG ACA GGA GTG TTC TCA-3′, and probe, 5′-CAG CCT CTC CAC TTT CAT CTA TAG CAT CTC C-3′ (FAM, TAMRA); mIL-24, forward, 5′-GCT CTC CAT GCC ATT TCA A-3′, reverse, 5′-TGG CCA AGG GTC TGA AGT-3′, and probe, 5′-TGT ACA TCC CTG CTG TCC TCA AGG C-3′ (FAM, TAMRA); mIL-6, forward, 5′-TCC AAT GCT CTC CTA ACA GAT AAG-3′, reverse, 5′-CAA GAT GAA TTG GAT GGT CTT G-3′, and probe, 5′-TCC TTA GCC ACT CCT TCT GTG ACT CCA-3′ (FAM, TAMRA); mS100A8, forward, 5′-TGT CCT CAG TTT GTG CAG AAT ATA AA-3′, reverse, 5′-TCA CCA TCG CAA GGA ACT CC-3′, and probe 5′-CGA AAA CTT GTT CAG AGA ATT GGA CAT CAA TAG TGA-3′ (FAM, TAMRA); mS100A9, forward, 5′-GGT GGA AGC ACA GTT GGC A-3′, reverse, 5′-GTG TCC AGG TCC TCC ATG ATG-3′, and probe, 5′-TGA AGA AAG AGA AGA GAA ATG AAG CCC TCA TAA ATG-3′ (FAM, TAMRA); mRegIIIγ, forward, 5′-ATG GCT CCT ATT GCT ATG CC-3′, reverse, 5′-GAT GTC CTG AGG GCC TCT T-3′, and probe, 5′-TGG CAG GCC ATA TCT GCA TCA TAC C-3′ (FAM, TAMRA); mPAP/HIP/RegIIIβ, forward, 5′-ATG GCT CCT ACT GCT ATG CC-3′, reverse, 5′-GTG TCC TCC AGG CCT CTT T-3′, and probe, 5′-TGA TGC AGA ACT GGC CTG CCA-3′ (FAM, TAMRA); mIL-12p40, forward, 5′-ACA TCT ACC GAA GTC CAA TGC A-3′, reverse, 5′-GGA ATT GTA ATA GCG ATC CTG AGC-3′, and probe, 5′-TGC ACG CAG ACA TTC CCG CCT-3′ (FAM, TAMRA); mIL-23p 19, forward, 5′-GGT GGC TCA GGG AAA TGT-3′, reverse, 5′-GAC AGA GCA GGC AGG TAC AG-3′, and probe, 5′-CAG ATG CAC AGT ACT CCA CAC AGC AGC-3′ (FAM, TAMRA); mIL-20Rβ, forward, 5′-CAG GTG CTT CCA GTC CGT CT-3′, reverse, 5′-CTC TCC TGG AAT CCC CAA AGT-3′, and probe, 5′-CAG CAC AGA TGC CAA CGG CCT CAT-3′ (FAM, TAMRA); mIL-20Rα, forward, 5′-CTG GCC GCT TCG GGA CGC-3′, reverse, 5′-AAC CAC AGA ACA CAC AAG GAA CTG-3′, and probe, 5′-TCT GCT GCT GGC CGC TTC GG-3′ (FAM, TAMRA); mIL-22R, forward, 5′-GCT GGA CTC CCT TGT GTG T-3′, reverse, 5′-CAC ATG GCC TCA GTC TCA A-3′, and probe, 5′-CGC GGG ACC CTC ATC CTT TG-3′ (FAM, TAMRA); mIL-10Rβ, forward, 5′-TCC ACA GCA CCT GAA GGA GTT-3′, reverse, 5′-GGA GGG AAG GAG AAC AGC AGA-3′, and probe, 5′-TGG GCC ACC CCC ATC ACA GC-3′ (FAM, TAMRA). Reactions were run in duplicates and samples were normalized to the control housekeeping gene lPL-19 and reported according to the [symbol] Ct method: [symbol] Ct= [symbol] Ctₛₐₘₚₗₑ− [symbol] Ct_{reference}.

Ig ELISA

Analyses were performed on serum from collected whole blood as previously described (10). Briefly, ELISA plates (Nunc) were coated with heat-killed C. rodentium or with a goat anti-mouse Ig capture Ab diluted 1/1000 in PBS (SouthernBiotech). Coated plates were washed in PBS plus 0.05% Tween 20, blocked for 1 h with 300 μl of blocking buffer (PBS+0.5% BSA+10 PPM Proclin), and washed before addition of serially diluted standards (mouse monoclonal IgA, IgG, IgG3, and IgM from SouthernBiotech; IgG1, IgG2a, and IgG2b isotypes from Sigma-Aldrich; mouse IgG2c obtained from Bethyl Laboratories) or unknowns. Samples were incubated for 4 hours at room temperature. Plates were washed five times and the Ig isotypes were detected with goat anti-mouse IgA, IgM, IgG, IgG1, IgG2a, IgG2b, IgG2c, and IgG3 (SouthernBiotech) conjugated to horseradish peroxidase (HRP), diluted 1/4,000 in assay diluent (PBS+0.5% BSA+0.05% Tween 20+10 PPM Proclin, pH 7.4), and incubated for 1 hour at room temperature. After washing, TMB peroxidase substrate was added to each well and allowed to develop for 15 minutes, then stop solution (1 M Phosphoric acid) were added to each well. Absorbance was read at 450 nm in a Molecular Devices (Sunnyvale, Calif.) plate reader at OD₄₅₀.

In Vitro Colon Culture

Colons were removed from C57Bl/6 mice. After cleaning with cold PBS, colons were cut longitudinally. Colons were placed in a 100 mm Petri dish with 10 ml HBSS (Mediatech) buffer containing 2.5 μg/ml of Fungizone-Amphotericin B, 10 μg/ml Gentamicin, 100 U/ml Penicillin and 100 μg/ml Streptomycin (all from GIBCO, Invitrogen). Colons were gently scraped to remove mucus at the edge of the Petri dish and were transferred to a new Petri dish with fresh HBSS buffer. Colons were cut into 1-2 mm pieces and transferred to a 24-well plate with 50 mg colons/1 ml/well in RPMI buffer containing 10% heat inactivated FCS (HyClone), 2.5 μg/ml of Fungizone-Amphotericin B, 10 μg/ml Gentamicin, 2 mM L-Glutamine, 100 U/ml Penicillin and 100 μg/ml Streptomycin. 10 μg/ml of IL-22 (R & D systems) were added to the culture and incubated in 37° C. for 24 hours.

Microarray Analysis

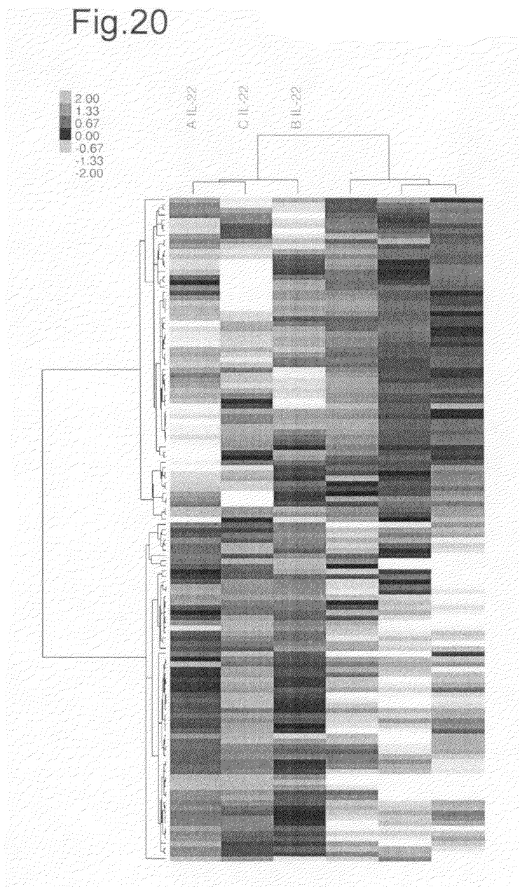

Quantity and quality of total RNA samples was determined using an ND-1000 spectrophotometer (Nanodrop Technologies) and Bioanalyzer 2100 (Agilent Technologies), respectively. The method for preparation of Cy-dye labeled cRNA and array hybridization was provided by Agilent Technologies. Briefly, total RNA sample was converted to double-stranded cDNA and then to Cy-dye labeled cRNA using Agilent's Low RNA Input Fluorescent Linear Amplification Kit. The labeled cRNA was purified using RNeasy mini kit (Qiagen). cRNA yield and Cy-dye incorporation was determined using ND-1000 spectrophotometer. 750 ng of the labeled cRNA was fragmented and hybridized to the Agilent's Whole Mouse Genome array as described in manufacturer's In situ Hybridization kit-plus. All samples were labeled with Cy5 and hybridized against Cy3 labeled universal mouse reference (Stratagene). Following hybridization, the arrays were washed, dried and scanned on Agilent's DNA microarray scanner. Agilent's Feature Extraction software 8.5 was used to analyze acquired array images. For microarray data clustering (FIG. 20), expression data was processed to Agilent log-ratio data by standard methods. Selected genes were clustered by iterative agglomeration of vectors most highly linked by Pearson correlation coefficient, with data for agglomerated vectors summarized by average linkage.

In Vitro Mouse Tail Tip Fibroblast Culture and Stimulation

To establish tail tip fibroblasts (TTFs), the tails from IL-17RC^{-/-} adult mice and wild type littermates were peeled, minced into 1 cm pieces, placed on culture dishes, and incubated in high glucose DMEM (containing 10% FCS, 2 nm glutamine, 100 U/ml Penicillin and 100 μg/ml Streptomycin) for 5 days. Cells that migrated out of the graft pieces were transferred to new plates (passage 2) and maintained in the same media. We used TTFs at passage 3 for stimulation experiments. TTFs were seeded into 24-well plate at a density of 1.2×10⁵ per well. Twenty four hours after seeding, recombinant murine IL-17A and IL-17F (R&D Systems) were added to the culture medium at various concentrations. Cell culture supernatant was harvested 24 hours after addition of cytokines and levels of murine IL-6 was measured by enzyme linked immunosorbent assay (ELISA) by mouse IL-6 ELISA set (BD Biosciences) following manufacturer's instructions.

Blockade of Murine IL-22 In Vivo

Blocking anti-mouse IL-22 (Clone 8E11, isotype mouse IgG1) mAb (11) was intraperitoneally injected before (Day 0) or 8 days after (Day 8) C. rodentium infection at a dose of 150 μg/mouse every other day. Certain control group also received isotype control IgG1 mAb.

Statistics

Statistical significance was calculated by one-way or two-way ANOVA using Prism software (GraphPad). All p values≦0.05 are considered significant, and are indicated in the text. Unless otherwise specified, all studies for which data are presented are representative of at least two independent experiments.

Although the foregoing invention has been described in some detail by way of illustration and example for purposes of clarity of understanding, the descriptions and examples should not be construed as limiting the scope of the invention. The disclosures of all patent and scientific literatures cited herein are expressly incorporated in their entirety by reference.

### Example 10

### The LT Pathway is Mediated by IL-22 During Citrobacer rodentium Infection

To help determine if IL-22 is important for the mortality caused by LT blockade, we performed a rescue experiment in which we expressed IL-22 in the mouse at the same time of LTbR-Fc treatment. The method we used for IL-22 expression was hydrodynamic tail vein delivery of plasmid DNA encoding mouse IL-22. Human LTbR-Ig was constructed as follows: human LTbR encompassing the extracellular domain (position 1 through position 224; SEQ ID NO:57) was cloned into a modified pRK5 expression vector encoding the human IgG1 Fc region (SEQ ID NO:58) downstream of the LTbR sequence. Proteins were overexpressed in CHO cells and purified by protein A affinity chromatography. Murine LTbR.Ig was constructed as follows: murine LTbR encompassing the extracellular domain (position 1 through position 222; SEQ ID NO:59) was cloned into a modified pRK5 expression vector encoding the murine IgG2a Fc region (SEQ ID NO:60) downstream of the LTbR sequence.

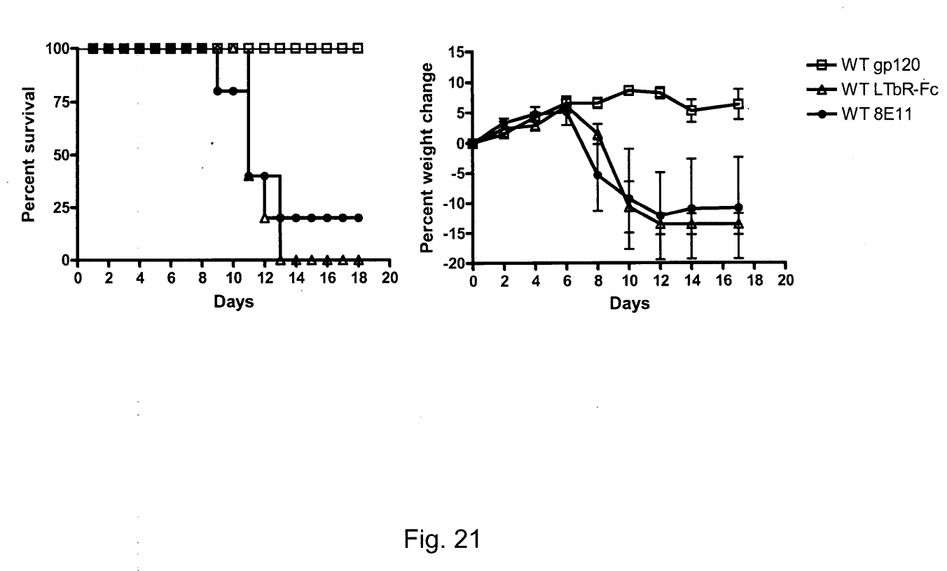

In FIG. 21, we find that LTbR-Fc produces a similar weight loss curve (FIG. 21 right panel) and death curve. (FIG. 21 left panel) to IL-22 blockade which led us to examine the relationship between LT and IL-22. C. rodetium infection leads to early expression of IL-22 in the colon.

FIG. 21 shows the percent survival of mice inoculated with Citrobacter rodentium. 6-8 week old Balb/c mice were fasted for 8 h before oral inoculation with 2×109 C. rodentium strain DBS100 (ATCC 51459; American Type Culture Collection) in a total volume of 200 μl per mouse. While fasting, animals had access to water. Inoculation and all subsequent manipulations were conducted in BL-2 biosafety cabinets. Animals were allowed access to food after inoculation. Bacteria were prepared by incubation with shaking at 37° C. overnight in LB broth. The relative concentration of bacteria was assessed by measuring absorbance at OD600 and each inoculation culture was serially diluted and plated to confirm CFU administered. On the day of inoculation, mice were also injected with 150 ug of anti-gp120 mAb, anti-IL-22 8E11 mAb, or LTbR-Fc 3 times per week.

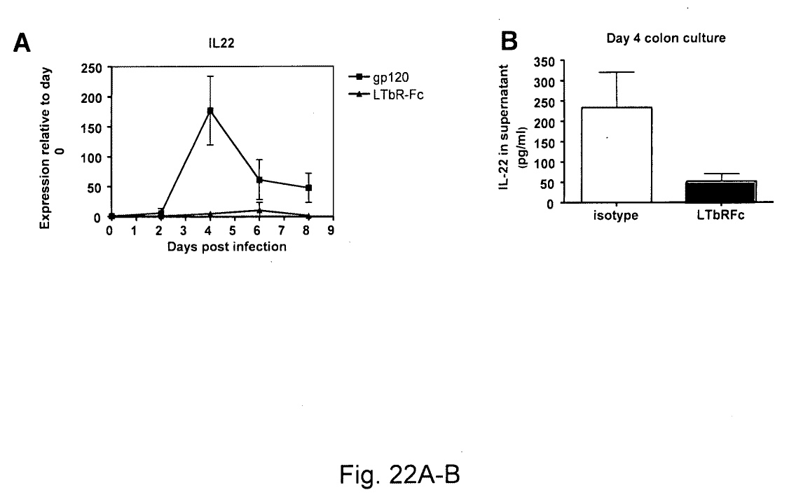

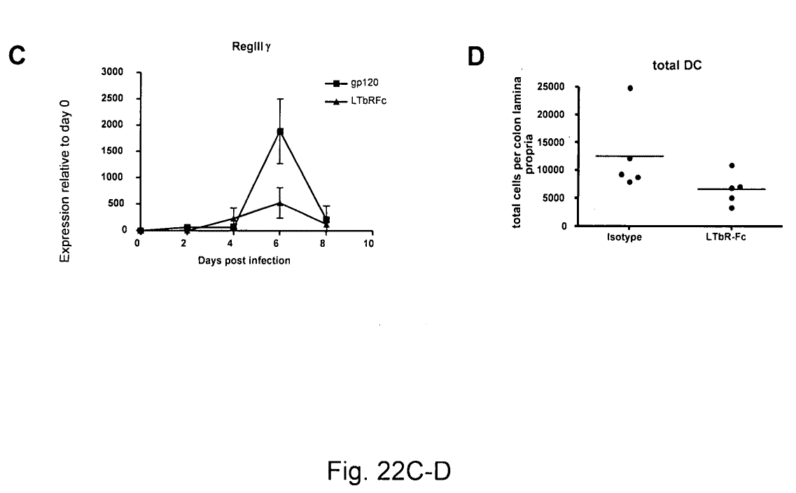

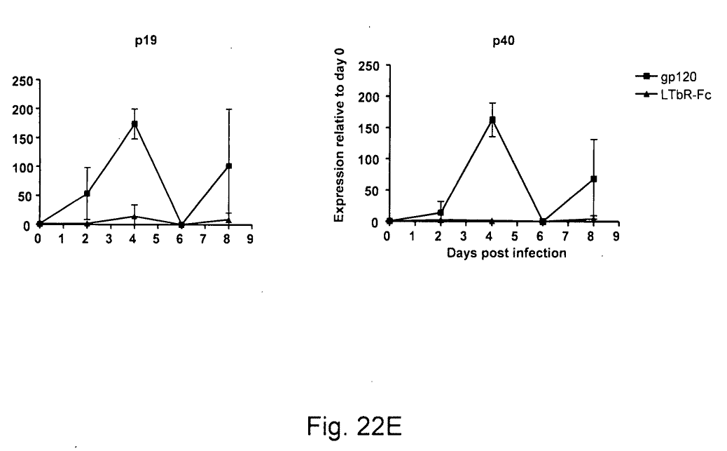

LT pathway regulate multiple upstream aspects that important for IL-22 production. FIG. 22 provides data on the LT pathway after infection with C. rodentium. A, C, E. Colons were harvested at different timepoints after infection with C. rodentium. Mice were injected with 150 ug anti-gp120 or LTbR-Fc every other day. RNA was extracted using Qiagen RNeasy Kit. Taqman analysis was performed to determine expression of IL-22, RegIIg, p19, or p40 relative to the day 0 timepoint. B. On day 4 after infection, colons were collected. After cleaning with cold PBS, colons were cut longitudinally. Colons were placed in a 100 mm Petri dish with 10 ml HBSS (Mediatech) buffer containing 2.5 μg/ml of Fungizone-Amphotericin B, 10 μg/ml Gentamicin, 100 U/ml Penicillin and 100 μg/ml Streptomycin (all from GIBCO, Invitrogen). Colons were gently scraped to remove mucus at the edge of the Petri dish and were transferred to a new Petri dish with fresh HBSS buffer. Colons were cut into 1-2 mm pieces and transferred to a 24-well plate with 50 mg colons/1 ml/well in RPMI buffer containing 10% heat inactivated FCS (HyClone), 2.5 μg/ml of Fungizone-Amphotericin B, 10 μg/ml Gentamicin, 2 mM L-Glutamine, 100 U/ml Penicillin and 100 μg/ml Streptomycin. 10 ng/ml of rmIL-22 (R & D systems) were added to the culture and incubated in 37° C. for 24 hours. Supernatants were collected for an IL-22 ELISA. D. Day 6 colon lamina propria cells. DC determined by CD11c+ and MHC II+. Colons were harvested and flushed with HBSS without calcium and magnesium (Invitrogen) with 2% FBS and 10 mM HEPES. Colons were cut longitudinally, and then sectioned into 2-4 cm pieces, and the pieces were transferred to a 10 cm dish with HBSS without calcium and magnesium, 2% FBS, 1 mM EDTA, 10 mM HEPES, and 1 mM DTT (Sigma-Aldrich). IEL fractions were collected and discarded after a 45 minute incubation at 37° C. while shaking at 200 rpm. For LPMCs isolation, the remaining epithelial layer was peeled off and the colon pieces were diced and placed into RPMI containing 10% FCS, 20 mM HEPES, and 0.5 mg/ml collagenase/dispase (Roche Diagnostics). Colon pieces were incubated for one hour at 37° C. while shaking. Isolated epithelial cells were washed and used for FACS analysis.

In FIG. 22, we find that LTbR-Fc blocked the induction of IL-22 as well as RegIIIg which has been shown to be induced by IL-22 (FIG. 22A-C). Dendritic cells have previously been shown to produce IL-22 and we find a slight reduction of DC numbers in the lamina propria of the colon 6 days after infection (FIG. 22D). The decrease in IL-22 caused by LTbR-Fc is most likely due the loss of IL-23, since both p19 and p40 expression is inhibited after LTbR-Fc treatment during infection (FIG. 22E).

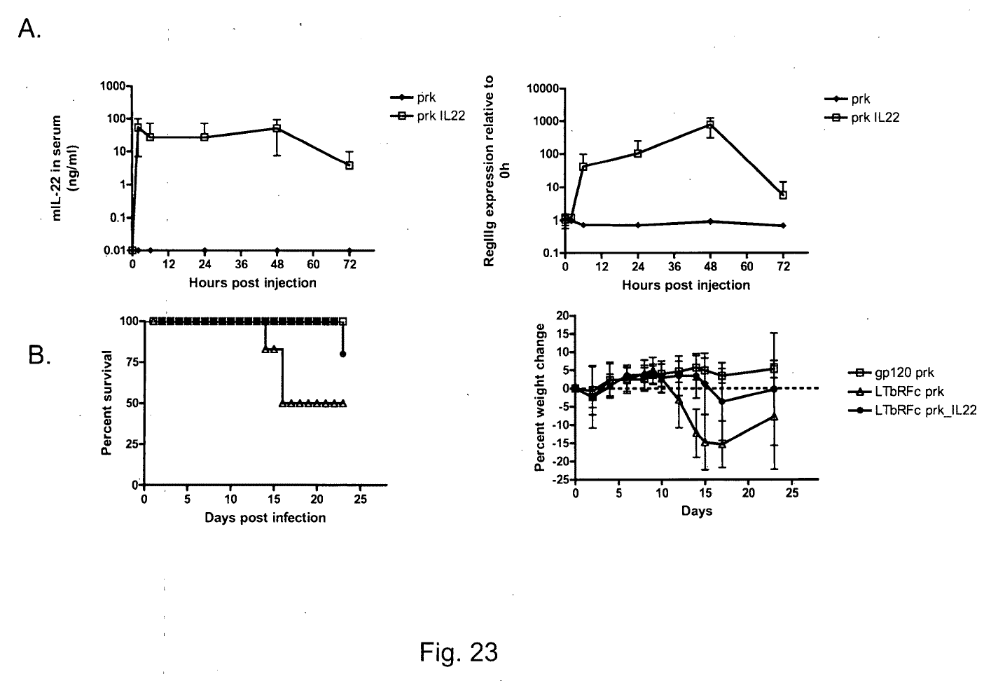

IL-22 partially rescues the defects seen in LTbR treated mice. FIG. 23 provides data concerning the effect of IL-22 on LTbR treated mice. A. Test of expression of IL-22 in serum and RegIIIg in colon after tail vein injection of IL-22 plasmid. B. Rescue of LTbRFc effects with IL-22 plasmid.

On day-1, animals were weighed and grouped, extra mice were euthanized. After weighing, all animals were fasted 14 h. The next morning (day 0), all mice were orally inoculated with 2-4×10e9 CFU of C. rodentium in 200 ul PBS. 150 ug control mAb or Fc fusion protein was injected i.p. in 200 ul PBS three times per week for two weeks starting on the same day as bacteria inoculation. Food was replaced back by investigators after inoculation. Six hours later plasmid DNA was injected by tail vein. Tail vein injection experiments: 1) DNA construct (pRK vector or pRK-mIL-22) was diluted in Ringer's to a concentration to yield a final dose of 10 micrograms/mouse/injection. 2) Each mouse was injected intravenously in the tail vein with approximately 1.6 ml of the solution containing DNA in Ringer's. 3) Doses were administered as a bolus intravenous injection (tail vein) over a period of 4-5 seconds (8 seconds maximum) for maximum DNA uptake. Mice were restrained without anesthesia in a conical acrylic restrainer with a heating element to increase body temperature and dilate blood vessels. 4) Disposable sterile syringes were used for each animal. Animals were continuously monitored until they are clinically normal. 5) Animals were observed for any adverse clinical signs for at least 20 minutes post dose. If animals were not clinically normal by 1 hour post dose, they were euthanized or they were monitored until they were clinically normal. Moribund animals were euthanized. All manipulations were performed in BL-2 biosafety cabinets. During infection, moribund animals or those showing unalleviated distress or rectal prolapse were euthanized.

The mice were monitored for 4 weeks everyday. Between day 5 to day 17 when LTbR-Fc treated mice might become moribund, the mice were monitored twice per day including weekends. Fecal pellets were collected every week to measure CFU of C. rodentium. Mice were weighed once per week during the study. If mice exhibited a weight loss of 15% or more, they were weighed daily. If the weight loss exceeds 20%, the mice were euthanized. At the end of the study, all mice were euthanized and spleen, and colon were collected for histology, RNA or FACS analysis.

As shown in FIG. 23A, we can detect expression of IL-22 in the mouse serum beginning at 2 hours post-injection, with expression declining at 72 hours. We can also detect expression of RegIIIg in the colon, suggesting that active IL-22 can act on the colon when expressed in this manner. IL-22 could partially rescue mortality and weight loss induced by LTb-Fc treatment during infection (FIG. 23B).

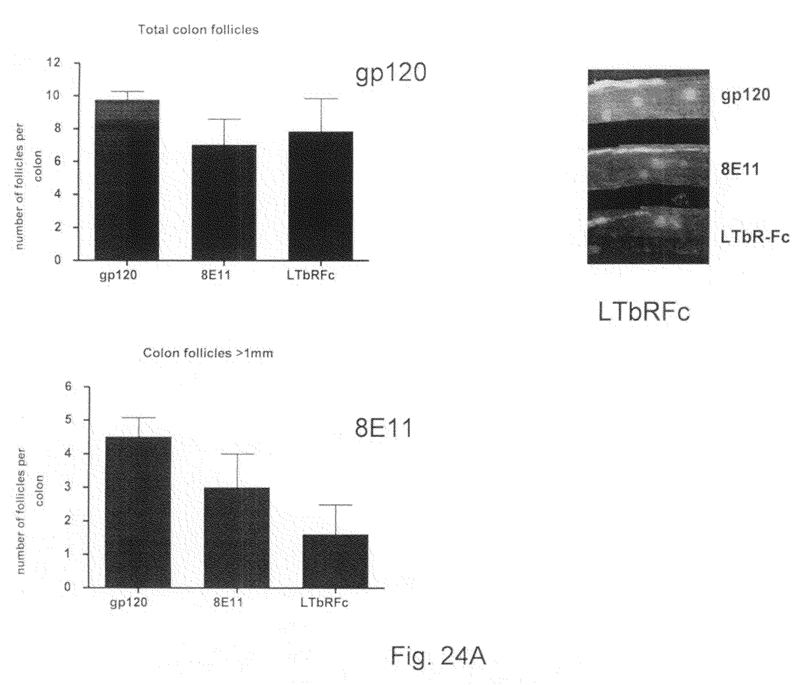

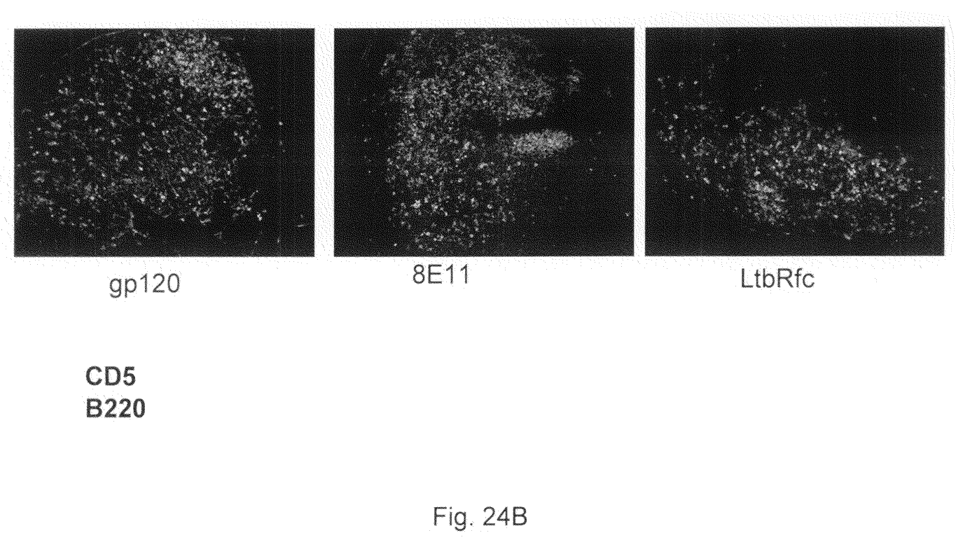

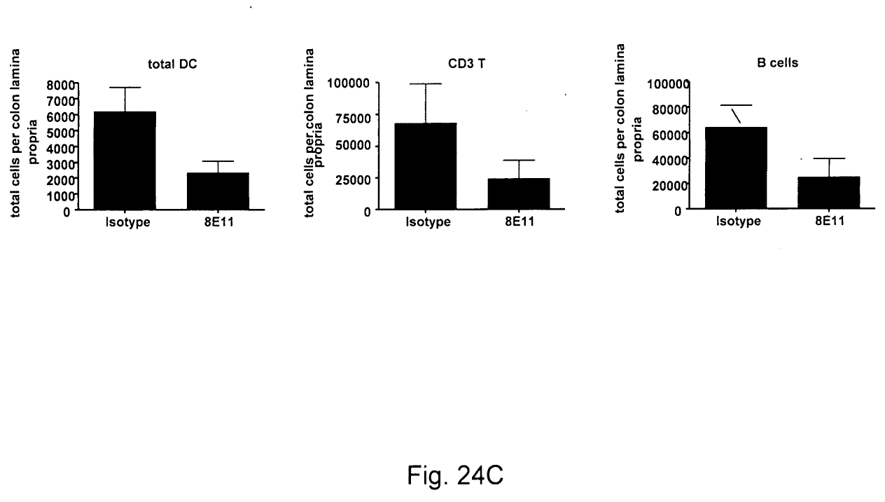

Treatment of mice with IL-22 mAb (8E11). FIG. 24 shows data demonstrating that treatment with IL-22 mAb 8SE11 leads to reduced colon follicles, compromised B/T cell organization, and reduced DC, T cell, and B cell numbers in the colon. A. Six days after infection, colons were harvested and cut longitudinally. After a 30 minute incubation in HBSS without calcium and magnesium, 2% FBS, 1 mM EDTA, 10 mM HEPES, and 1 mM DTT, colons were gently scraped to remove epithelial cells. Follicles were identified as white, round masses. There were five mice per group and each colon was counted and plotted as total follicles found or total follicles greater than 1 mm found. B. Six days after infection, colons were flushed with cold PBS and quick frozen in OCT. Six micron sections were cut, dried, then fixed in acetone. Sections were blocked with 10% serum, the incubated with anti-CD5 FITC and anti-B220 APC at 10 ug/ml each. Images were capture on a NIKON BX61 microscope. C. Six days after infection colon lamina propria cells were isolated at described above. FAC analysis was performed to determine the number of dendritic cells, CD3 T cells, and B cells after 8E11 treatment.

As shown in FIG. 24, we treated mice with either IL-22 blocking antibody or LTbR-Fc and counted lymphoid follicles in the colon after six days post infection in order to determine if IL-22 could have a role in formation of colon lymphoid structures. We found a decrease in follicles greater than 1 mm, suggesting IL-22 and LT could be important for the increase in follicle size after infection (FIG. 24A). Histological analysis shows that blocking IL-22 disrupted the T and B cell zones of the follicle while LT blockade had a similar effect (FIG. 24B). We next determined whether blockade of IL-22 leads to a change in cell numbers in the colon lamina propria. We found that IL-22 blockade led to decreases in DC, T cell, and B cell numbers during infection. In conclusion, IL-22 appears to be important for lymphoid follicle formation and may be an important downstream component of the lymphotoxin pathway in the colon.

What is claimed is:

1. A method of treating an infection by a microbial pathogen, in a subject, by modulating an anti-microbial immune response in said subject, comprising administering to said subject an effective amount of an anti-microbial polypeptide (AMP), wherein said AMP is IL-22.

2. A method of treating an infection by a microbial pathogen, in a subject, by modulating an anti-microbial immune response in said subject, comprising administering to said subject an effective amount of an anti-microbial polypeptide (AMP) or modulator thereof, wherein said AMP is selected from a group consisting of: IL-6, IL-18, IL-23, REG Iα, REG Iβ, HIP/PAP, REG III, REG IV, Reg-related sequence (RS) and LT.

3. A method of modulating the activity of an anti-microbial polypeptide (AMP) in cells of a subject infected with a microbial pathogen, comprising contacting said cells with an isolated AMP, wherein said AMP is IL-22.

4. A method of modulating the activity of an anti-microbial polypeptide (AMP) in cells of a subject infected with a microbial pathogen, comprising contacting said cells with an isolated AMP, wherein said AMP is selected from a group consisting of: IL-6, IL-18, IL-23, REG Iα, REG Iβ, HIP/PAP, REG III, REG IV, Reg-related sequence (RS) and LT.

5. The method of claim 1, wherein said infection is a microbial disorder.

6. The method of claim 5, wherein said microbial disorder is Inflammatory Bowel Disease (IBD).

7. The method of claim 5, wherein said microbial disorder is Crohn's or ulcerative colitis (UC).

8. The method of claim 1, wherein said microbial pathogen is a bacteria.

9. The method of claim 8, wherein said bacteria is gram negative.

10. The method of claim 8, wherein said bacteria is gram positive.

11. The method of claim 8, wherein said bacteria is an attaching or effacing (A/E) bacteria.

12. The method of claim 11, wherein said attaching or effacing (A/E) bacteria is an enterohemorrhagic Escherichia coli (EHEC) and enteropathogenic E. Coli (EPEC).

13. The method of claim 12, wherein said enteropathogenic E. coli (EHEC) is E. coli 0157:H7 or E. coli 055:H7.

14. The method of claim 2, wherein said anti-microbial polypeptide (AMP) is RegIIIβ and RegIIIγ.

15. The method of claim 1, wherein said microbial pathogen is a virus.

16. A method of treating an infection by a microbial pathogen, in a subject, by modulating an anti-microbial immune response in said subject, comprising administering to said subject an effective amount of an anti-microbial polypeptide (AMP) modulator, wherein said AMP modulator is an IL-22 agonist.

17. The method of claim 16, wherein said agonist increases expression and/or activity of said IL-22.

18. The method of claim 16, wherein said agonist is a polypeptide or nucleic acid molecule.

19. The method of claim 16, wherein said agonist is a fusion polypeptide.

20. The method of claim 16, wherein said agonist is an Fc fusion polypeptide.

21. The method of claim 16, wherein said agonist is an antibody or biologically active fragment thereof.

22. The method of claim 16, wherein said agonist is a monoclonal antibody.

23. The method of claim 16, wherein said agonist is a humanized antibody.

24. The method of claim 1 or 3 wherein the amino acid sequence of said IL-22 comprises a sequence shown as SEQ ID NO:8.

25. The method of claim 2 or 4 wherein the amino acid sequence of said AMP comprises a sequence selected from a group of amino acid sequences consisting of SEQ ID NO: 2, SEQ ID NO: 4, SEQ ID NO: 6, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 50, and SEQ ID NO: 52.

26. The method of claim 1 or 3, wherein the nucleic acid sequence encoding said IL-22 is a sequence shown as SEQ ID NO:7.

27. The method of claim 1 or 3, wherein the nucleic acid sequence encoding said AMP comprises a sequence selected from a group of nucleic acid sequences consisting of SEQ ID NO: 1, SEQ ID NO: 3, SEQ ID NO: 5, SEQ ID NO: 9, SEQ ID NO:11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 49, and SEQ ID NO: 51.

28. A kit comprising a pharmaceutical composition for treatment of a microbial disorder, wherein said pharmaceutical composition comprises an anti-microbial polypeptide (AMP), wherein said AMP is IL-22.

29. A kit comprising one or more pharmaceutical compositions for treatment of a microbial disorder, wherein said pharmaceutical compositions each comprise a different anti-microbial polypeptide (AMP) or modulator thereof, and wherein said AMP is selected from a group consisting of: IL-6, IL-18, IL-23, REG Iα, REG Iβ, HIP/PAP, REG III, REG IV, Reg-related sequence (RS), and LT.


SEQUENCE LISTING (118 sequences)

SEQ ID NO: 1 - DNA, 1131 bp, Homo sapiens
cattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagca

SEQ ID NO: 2 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - DNA, 2347 bp, Homo sapiens
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatc

SEQ ID NO: 4 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - DNA, 1145 bp, Homo sapiens
attctctccccagcttgctgagccctttgctcccctggcgactgcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatacaaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtg

SEQ ID NO: 6 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 7 - DNA, 1147 bp, Homo sapiens
cgaccaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgacctcaataaacacttggatatcc

SEQ ID NO: 8 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 9 - DNA, 1049 bp, Homo sapiens
aaaacaacaggaagcagcttacaaactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggctaaaaaaaa

SEQ ID NO: 10 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 11 - DNA, 808 bp, Homo sapiens
gatataaagctcctacagctacctggcctgagaagccaactcagactcagccaacagagattgttgatttgcctcttaagcaagagattcattgcagctcagcatggctcagaccagctcatacttcatgctgatctcctgcctgatgtttctgtctcagagccaaggccaagaggcccagacagagttgccccaggcccggatcagctgcccagaaggcaccaatgcctatcgctcctactgctactactttaatgaagaccgtgagacctgggttgatgcagatctctattgccagaacatgaattcgggcaacctggtgtctgtgctcacccaggccgagggtgcctttgtggcctcactgattaaggagagtggcactgatgacttcaatgtctggattggcctccatgaccccaaaaagaaccgccgctggcactggagcagtgggtccctggtctcctacaagtcctggggcattggagccccaagcagtgttaatcctggctactgtgtgagcctgacctcaagcacaggattccagaaatggaaggatgtgccttgtgaagacaagttctcctttgtctgcaagttcaaaaactagaggcaactggaaaatacatgtctagaactgatccagcaattacaacggagtcaaaaattaaaccggaccatctctccaactcaactcaacctggacactctcttctctgctgagtttgccttgttaatcttcaatagttttacctaccccagtctttggaaccctaaataataaaaataaacatgtttccactattgtgctgtc

SEQ ID NO: 12 - protein, 166 aa, Homo sapiens
MAQTSSYFMLISCLMFLSQSQGQEAQTELPQARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFQKWKDVPCEDKFSFVCKFKN

SEQ ID NO: 13 - DNA, 773 bp, Homo sapiens
aagccacctcaagtggacaaggcacttaccaacagagattgctgatttgctccttaagcaagagattcactgccgctaagcatggctcagaccaactcgttcttcatgctgatctcctccctgatgttcctgtctctgagccaaggccaggagtcccagacagagctgcctaatccccgaatcagctgcccagaaggcaccaatgcctatcgctcctactgctactactttaatgaagaccctgagacctgggttgatgcagatctctattgccagaacatgaattcaggcaacctggtgtctgtgctcacccaggcggagggtgccttcgtggcctcactgattaaggagagtagcactgatgacagcaatgtctggattggcctccatgacccaaaaaagaaccgccgctggcactggagtagtgggtccctggtctcctacaagtcctgggacactggatccccgagcagtgctaatgctggctactgtgcaagcctgacttcatgctcaggattcaagaaatggaaggatgaatcttgtgagaagaagttctcctttgtttgcaagttcaaaaactagaggaagctgaaaaatggatgtctagaactggtcctgcaattactatgaagtcaaaaattaaactagactatgtctccaactcagttcagaccatctcctccctaatgagtttgcatcgctgatcttcagtaccttcacctgtctcagtctctagagccctgaaaaataaaaacaaacttatttttatccag

SEQ ID NO: 14 - protein, 166 aa, Homo sapiens
MAQTNSFFMLISSLMFLSLSQGQESQTELPNPRISCPEGTNAYRSYCYYFNEDPETWVDADLYCQNMNSGNLVSVLTQAEGAFVASLIKESSTDDSNVWIGLHDPKKNRRWHWSSGSLVSYKSWDTGSPSSANAGYCASLTSCSGFKKWKDESCEKKFSFVCKFKN

SEQ ID NO: 15 - DNA, 807 bp, Homo sapiens
aaaccataccatatcccaccagagagtgactcctgattgcctcctcaagtcgcagacactatgctgcctcccatggccctgcccagtgtatcttggatgctgctttcctgcctcatgctgctgtctcaggttcaaggtgaagaaccccagagggaactgccctctgcacggatccgctgtcccaaaggctccaaggcctatggctcccactgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtcatacggcctaaggctttaaagagcaataaaatttttagtctgc

SEQ ID NO: 16 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 17 - DNA, 784 bp, Homo sapiens
aaaccataccatatcccaccagagagtcgcagacactatgctgcctcccatggccctgcccagtgtatcttggatgctgctttcctgcctcatgctgctgtctcaggttcaaggtgaagaaccccagagggaactgccctctgcacggatccgctgtcccaaaggctccaaggcctatggctcccactgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtcatacggcctaaggctttaaagagcaataaaatttttagtctgc

SEQ ID NO: 18 - protein, 175 aa, Homo sapiens; identical to SEQ ID NO: 16

SEQ ID NO: 19 - DNA, 1002 bp, Homo sapiens
gggagggtcccttcctcagggagcacaggaactctgagactcagcaagggtgtcctgggagggctcggggatgggagagtacacagattcacaactcattcagaactgtagaagatgatggatgtgaccaagatcactttagtcctaggggactagagaaggaaaatgacatgaggcagtggggtatctgtgtgttctcccactgaccacgctttctttagtgactcctgattgcctcctcaagtcgcagacactatgctgcctcccatggccctgcccagtgtatcttggatgctgctttcctgcctcatgctgctgtctcaggttcaaggtgaagaaccccagagggaactgccctctgcacggatccgctgtcccaaaggctccaaggcctatggctcccactgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtcatacggcctaaggctttaaagagcaataaaatttttagtctgc

SEQ ID NO: 20 - protein, 175 aa, Homo sapiens; identical to SEQ ID NO: 16

SEQ ID NO: 21 - DNA, 847 bp, Homo sapiens
ccatccctgagatctttttataaaaaacccagtctttgctgaccagacaaagcataccagatctcaccagagagtcgcagacactatgctgcctcccatggccctgcccagtgtgtcctggatgctgctttcctgcctcattctcctgtgtcaggttcaaggtgaagaaacccagaaggaactgccctctccacggatcagctgtcccaaaggctccaaggcctatggctccccctgctatgccttgtttttgtcaccaaaatcctggatggatgcagatctggcttgccagaagcggccctctggaaaactggtgtctgtgctcagtggggctgagggatccttcgtgtcctccctggtgaggagcattagtaacagctattcatacatctggattgggctccatgaccccacacagggctctgagcctgatggagatggatgggagtggagtagcactgatgtgatgaattactttgcatgggagaaaaatccctccaccatcttaaaccctggccactgtgggagcctgtcaagaagcacaggatttctgaagtggaaagattataactgtgatgcaaagttaccctatgtctgcaagttcaaggactagggcaggtgggaagtcagcagcctgagcttggcgtgcagctcatcatggacatgagaccagtgtgaagactcaccctggaagagaatattctccccaaactgccctacctgactaccttgtcatgatcctccttctttttcctttttcttcaccttcatttcaggcttttctctgtcttccatgtcttgagatctcagagaataataataaaaatgttactttatacttaaaaaa

SEQ ID NO: 22 - protein, 175 aa, Homo sapiens
MLPPMALPSVSWMLLSCLILLCQVQGEETQKELPSPRISCPKGSKAYGSPCYALFLSPKSWMDADLACQKRPSGKLVSVLSGAEGSFVSSLVRSISNSYSYIWIGLHDPTQGSEPDGDGWEWSSTDVMNYFAWEKNPSTILNPGHCGSLSRSTGFLKWKDYNCDAKLPYVCKFKD

SEQ ID NO: 23 - DNA, 947 bp, Homo sapiens
ccatccctgagatctttttataaaaaacccagtctttgctgaccagacaaagcataccagatctcaccagagagtcctaggggactacagaaggaaaaagacaagaggcagtaggatatctgtgtgtcctcccgctgaccacacttcctttagtgacccgattgcctcctcaagtcgcagacactatgctgcctcccatggccctgcccagtgtgtcctggatgctgctttcctgcctcattctcctgtgtcaggttcaaggtgaagaaacccagaaggaactgccctctccacggatcagctgtcccaaaggctccaaggcctatggctccccctgctatgccttgtttttgtcaccaaaatcctggatggatgcagatctggcttgccagaagcggccctctggaaaactggtgtctgtgctcagtggggctgagggatccttcgtgtcctccctggtgaggagcattagtaacagctattcatacatctggattgggctccatgaccccacacagggctctgagcctgatggagatggatgggagtggagtagcactgatgtgatgaattactttgcatgggagaaaaatccctccaccatcttaaaccctggccactgtgggagcctgtcaagaagcacaggatttctgaagtggaaagattataactgtgatgcaaagttaccctatgtctgcaagttcaaggactagggcaggtgggaagtcagcagcctgagcttggcgtgcagctcatcatggacatgagaccagtgtgaagactcaccctggaagagaatattctccccaaactgccctacctgactaccttgtcatgatcctccttctttttcctttttcttcaccttcatttcaggcttttctctgtcttccatgtcttgagatctcagagaataataataaaaatgttactttatacgtaaaaaa

SEQ ID NO: 24 - protein, 175 aa, Homo sapiens; identical to SEQ ID NO: 22

SEQ ID NO: 25 - DNA, 1285 bp, Homo sapiens
ataagacttttatggatggattgtttttctcaaataatattatcgctttgtgactaaagtaaagattattaattcctgaggcaagaagatataaaagctccagaaacgttgactgggaccactggagacactgaagaaggcaggggcccttagagtcttggttgccaaacagatttgcagatcaaggagaacccaggagtttcaaagaagcgctagtaaggtctctgagatccttgcactagctacatcctcagggtaggaggaagatggcttccagaagcatgcggctgctcctattgctgagctgcctggccaaaacaggagtcctgggtgatatcatcatgagacccagctgtgctcctggatggttttaccacaagtccaattgctatggttacttcaggaagctgaggaactggtctgatgccgagctcgagtgtcagtcttacggaaacggagcccacctggcatctatcctgagtttaaaggaagccagcaccatagcagagtacataagtggctatcagagaagccagccgatatggattggcctgcacgacccacagaagaggcagcagtggcagtggattgatggggccatgtatctgtacagatcctggtctggcaagtccatgggtgggaacaagcactgtgctgagatgagctccaataacaactttttaacttggagcagcaacgaatgcaacaagcgccaacacttcctgtgcaagtaccgaccatagagcaagaatcaagattctgctaactcctgcacagccccgtcctcttcctttctgctagcctggctaaatctgctcattatttcagaggggaaacctagcaaactaagagtgataagggccctactacactggcttttttaggcttagagacagaaactttagcattggcccagtagtggcttctagctctaaatgtttgccccgccatccctttccacagtatccttcttccctcctcccctgtctctggctgtctcgagcagtctagaagagtgcatctccagcctatgaaacagctgggtctttggccataagaagtaaagatttgaagacagaaggaagaaactcaggagtaagcttctagaccccttcagcttctacacccttctgccctctctccattgcctgcaccccaccccagccactcaactcctgcttgtttttcctttggccataggaaggtttaccagtagaatccttgctaggttgatgtgggccatacattcctttaataaaccattgtgtacataagaaaaaaaaaa

SEQ ID NO: 26 - protein, 158 aa, Homo sapiens
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 27 - DNA, 1087 bp, Mus musculus
ccaagaacgatagtcaattccagaaaccgctatgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctagtgcgttatgcctaagcatatcagtttgtggacattcctcactgtggtcagaaaatatatcctgttgtcaggtatctgacttatgttgttctctacgaagaactgacaatatgaatgttgggacactattttaattatttttaatttattgataatttaaataagtaaactttaagttaatttatgattgatatttattatttttatgaagtgtcacttgaaatgttatatgttatagttttgaaatgataacctaaaaatctatttgatataaatattctgttacctagccagatggtttcttggaatgtataagtttacctcaatgaattgctaatttaaatatgtttttaaagaaatctttgtgatgtatttttataatgtttagactgtcttcaaacaaataaattatattatattt

SEQ ID NO: 28 - protein, 211 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 29 - DNA, 1951 bp, Mus musculus
agaaggaacagtgggtgtccaggcacatcagaccaggcagctcgcagcaaagcaaggtaagttctctcctcttccctgtcgctaactccctgcatctagaggctgtccagattcagactccaggggacaggctacccctgaaccaggcagcgtgggagtgggatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctaggatgcaacgttggaaaggaaagaaaagtggaagacattaaggaagaaaaatttaaactcaggatggaagagtcccccaaaagctgtcttctgcttggttggctttttccagttttcctaagttcatcatgacacctttgctgatttctacatgtaaatgttaaatgcccgcagagccagggagctaatgtatgcatagatattctagcattccacttggccttatgctgttgaaatatttaagtaatttatgtatttattaatttatttctgcatttcacatttgtataccaagatgtattgaatatttcatgtgctcgtggcctgatccactgggaccaggccctattatgcaaattgtgagcttgttatcttcttcaacagctcttcaatcagggctgcgtaggtacattagcttttgtgacaaccaataagaacataatattctgacacaagcagtgttacatatttgtgaccagtaaagacataggtggtatttggagacatgaagaagctgtaaagttgactctgaagagtttagcactagtttcaacaccaagaaagactttttagaagtgatattgataagaaaccagggccttctttagaagggtacctaaatttaaaagaattttgaaaggctgggtatcggtggtatatgcttttaattccagcactcaggagaccaaggcaggcagatctctgtgagtttgaggacagcctggtgtacagagggagttccagcacagccagtgccacacagaaattctgtctcaaaaaca

SEQ ID NO: 30 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 31 - DNA, 866 bp, Mus musculus
ggcacagctggacctggtgggggttctctgtggttccatgctttctggactcctgcctgctggctggagctgctgacaggcctgacatcttctgcaacctccagcatcaggacaaagaaagccgcctcaaaccttccaaatcacttcctcttggcccaggaacaatggctgccatgtcagaagactcttgcgtcaacttcaaggaaatgatgtttattgacaacacgctttactttatacctgaagaaaatggagacctggaatcagacaactttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacatgtacaaagacagtgaagtaagaggactggctgtgaccctctctgtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagttaggtggggagggtttgtgttccagaaagatgattagcacacatgcgccttgtgatgacctcgcctgtatttccataacagaatacccgaggctgcatgatttatagagtaaacacgtttatttgt

SEQ ID NO: 32 - protein, 192 aa, Mus musculus
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 33 - DNA, 1121 bp, Mus musculus
cctaaacaggctctcctctcacttatcaactgttgacacttgtgcgatctctgatggctgtcctgcagaaatctatgagtttttcccttatggggactttggccgccagctgcctgcttctcattgccctgtgggcccaggaggcaaatgcgctgcccgtcaacacccggtgcaagcttgaggtgtccaacttccagcagccgtacatcgtcaaccgcacctttatgctggccaaggaggccagccttgcagataacaacacagacgtccggctcatcggggagaaactgttccgaggagtcagtgctaaagatcagtgctacctgatgaagcaggtgctcaacttcaccctggaagacgttctgctcccccagtcagacaggttccagccctacatgcaggaggtggtacctttcctgaccaaactcagcaatcagctcagctcctgtcacatcagcggtgacgaccagaacatccagaagaatgtcagaaggctgaaggagacagtgaaaaagcttggagagagtggagagatcaaggcgattggggaactggacctgctgtttatgtctctgagaaatgcttgcgtctgagcgagaagaagctagaaaacgaagaactgctccttcctgccttctaaaaagaacaataagatccctgaatggacttttttactaaaggaaagtgagaagctaacgtccatcatcattagaagatttcacatgaaacctggctcagttgaaaaagaaaatagtgtcaagttgtccatgagaccagaggtagacttgataaccacaaagattcattgacaatattttattgtcactgatgatacaacagaaaaataatgtactttaaaaaattgtttgaaaggaggttacctctcattcctttagaaaaaaagcttatgtaacttcatttccatatccaatattttatatatgtaagtttatttattataagtatacattttatttatgtcagtttattaatatggatttatttatagaaacattatctgctattgatatttagtataaggcaaataatatttatgacaataactatggaaacaagatatcttaggctttaataaacacatggatatcataaaaaaaaaa

SEQ ID NO: 34 - protein, 179 aa, Mus musculus
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 35 - DNA, 1359 bp, Mus musculus
cgcttagaagtcggactacagagttagactcagaaccaaaggaggtggatagggggtccacaggcctggtgcagatcacagagccagccagatctgagaagcagggaacaagatgctggattgcagagcagtaataatgctatggctgttgccctgggtcactcagggcctggctgtgcctaggagtagcagtcctgactgggctcagtgccagcagctctctcggaatctctgcatgctagcctggaacgcacatgcaccagcgggacatatgaatctactaagagaagaagaggatgaagagactaaaaataatgtgccccgtatccagtgtgaagatggttgtgacccacaaggactcaaggacaacagccagttctgcttgcaaaggatccgccaaggtctggctttttataagcacctgcttgactctgacatcttcaaaggggagcctgctctactccctgatagccccatggagcaacttcacacctccctactaggactcagccaactcctccagccagaggatcacccccgggagacccaacagatgcccagcctgagttctagtcagcagtggcagcgcccccttctccgttccaagatccttcgaagcctccaggcctttttggccatagctgcccgggtctttgcccacggagcagcaactctgactgagcccttagtgccaacagcttaaggatgcccaggttcccatggctaccatgataagactaatctatcagcccagacatctaccagttaattaacccattaggacttgtgctgttcttgttttgtttgttttgcgtgaagggcaaggacaccattattaaagagaaaagaaacaaaccccagagcaggcagctggctagagaaaggagctggagaagaagaataaagtctcgagcccttggccttggaagcgggcaagcagctgcgtggcctgaggggaagggggcggtggcatcgagaaactgtgagaaaacccagagcatcagaaaaagtgagcccaggctttggccattatctgtaagaaaaacaagaaaaggggaacattatactttcctgggtggctcagggaaatgtgcagatgcacagtactccagacagcagctctgtacctgcctgctctgtccctcagttctaacagaatctagtcactaagaactaacaggactaccaatacgaactgacaaatactaccactatgacctgtgacaaagctgcatatttattaagtgggaagggaacttttgatattatttatccttgtaacagtatagatgatggttatttattctatttataaggaattatgtattttttttttcaataaagatttatttatgtggc

SEQ ID NO: 36 - protein, 196 aa, Mus musculus
MLDCRAVIMLWLLPWVTQGLAVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 37 - DNA, 759 bp, Mus musculus
acaccatccagatctctggaagacagacaagatgctgcctccaacagcctgctccgtcatgtcctggatgctgctctcctgcctgatgctcttatctcaggttcaaggtgaagactccctgaagaatataccctccgcacgcattagttgccccaagggctcccaggcttatggctcctactgctatgccttgtttcagataccacagacctggtttgatgcagaactggcctgccaaaagaggcctggaggacacctcgtatctgtgctcaatagcgctgaggcttcattcttgtcctccatggtgaagagaacaggaaacagctaccaatacacttggattgggctccatgaccccactctgggtgcagaacccaatggaggtggatgggaatggagtaacaatgacgtgatgaattactttaactgggagaggaacccatctactgccttagaccgtgctttctgtggcagcttgtcaagagcttctggatttctaaaatggagagatatgacatgtgaggtgaagttgccctatgtctgcaaatttactggttaaacttatcagacagcaaacatcccgaatttgtcttgaagagcatcatggacaagggacaaaatgtgaagactcacctagaaaaagcattttctatctacagtccacattagagccttaatctgctctttccatatctgtctttagtccttttggtataagtttgggctcaattctaaaataaaaataagctttctgtcaca

SEQ ID NO: 38 - protein, 175 aa, Mus musculus
MLPPTACSVMSWMLLSCLMLLSQVQGEDSLKNIPSARISCPKGSQAYGSYCYALFQIPQTWFDAELACQKRPGGHLVSVLNSAEASFLSSMVKRTGNSYQYTWIGLHDPTLGAEPNGGGWEWSNNDVMNYFNWERNPSTALDRAFCGSLSRASGFLKWRDMTCEVKLPYVCKFTG

SEQ ID NO: 39 - DNA, 769 bp, Mus musculus
aagacaccttggtctcagcctgcagagatcgttgagttgcatcctaagcaaaagactgtctgctgctcagcatggctaggaacgcctacttcatcctgctctcatgcctgatcgtcctgtctccaagccaaggccaggaagctgaagaagacctgccatctgccaggatcagttgcccagaaggttccaatgcctacagctcctattgttactacttcactgaagaccgtttaacttgggctgatgcagatcttttttgccagaacatgaattcaggctacctggtgtcagttctcagtcaggctgagggcaactttgtggcctctctgattaaggagagtggcactacagacgccaatgtctggactggactccatgatcccaaaaggaatcgtcgctggcactggagcagtgggtctctgtttctctacaaatcctgggcaactgggtctcctaacagttccaatcgtggctactgtgtatctctgacttcaaacacaggatacaagaaatggaaggatgacaactgtgatgcccaatactcatttgtctgcaagttcaaaggctgaagtcacctgaaaaaaaatagtcatataaagcaaaattgaaattactatagtgtcagaaattaaattggaccatctatcaaaagcaaattagatcctgtcttcctggagagacattcttgcttcactgtcctatggtacctgtatctccattattttctggaaatttgcacaactgaaataaaaacacctttacaatgttg

SEQ ID NO: 40 - protein, 165 aa, Mus musculus
MARNAYFILLSCLIVLSPSQGQEAEEDLPSARISCPEGSNAYSSYCYYFTEDRLTWADADLFCQNMNSGYLVSVLSQAEGNFVASLIKESGTTDANVWTGLHDPKRNRRWHWSSGSLFLYKSWATGSPNSSNRGYCVSLTSNTGYKKWKDDNCDAQYSFVCKFKG

SEQ ID NO: 41 - DNA, 692 bp, Mus musculus
agtattcattattcctagctgacagaaattattgatttagaatttaaatcgaagactgtctgctgatcagcatggctcagaacaatgtataccttatcctgttcttatgcctgatgttcctgtcatacagccaaggccaggtagctgaagaagacttccccttggctgaaaaagaccttccttctgccaaaatcaactgcccagagggtgccaacgcctatggttcctactgttattatctaattgaagaccgtttgacctggggggaggctgatctcttttgccagaacatgaatgcaggtcacctggtgtcaatactcagccaggctgagagcaactttgtggcctcgctggttaaggagagtggtactacagcttccaatgtctggactggacttcatgaccctaaaagtaaccgtcgttggcactggagcagtggctccctatttctcttcaagtcatgggccactggagctccaagcactgccaaccgtggttattgtgtatcgctgacttcaaacacagcatacaaaaaatggaaggacgaaaactgtgaggcacagtactcctttgtctgcaagttcagagcctaaagtcacctgaagaacagatgtgcagaactctgttactatatattataaatgaaagaagaccatctatctgcaaaataaatactctttcctgtaataact

SEQ ID NO: 42 - protein, 173 aa, Mus musculus
MAQNNVYLILFLCLMFLSYSQGQVAEEDFPLAEKDLPSAKINCPEGANAYGSYCYYLIEDRLTWGEADLFCQNMNAGHLVSILSQAESNFVASLVKESGTTASNVWTGLHDPKSNRRWHWSSGSLFLFKSWATGAPSTANRGYCVSLTSNTAYKKWKDENCEAQYSFVCKFRA

SEQ ID NO: 43 - DNA, 837 bp, Mus musculus
gcccttccttcaaatcctatcataaagcagtcacctttgtcctgacaaaccatctcagatctctacaagagagacaagatgctgcctcacctggtcctcaacagtatttcctggatgctgctctcctgcctgttgtttgtatttcaggtacaaggtgaagacttccagaaggaagtgccctctccacgtaccagctgccccatgggttacaaggcttatcgctcccactgctatgccttagttatgacacctaaatcctggtttcaagcagatctggtctgccagaagagaccctcaggacatctcgtgtctattcttagtggaggtgaggcttcctttgtgtcctccttggtgaacggcagagtggacaactaccaagacatctggattgggctccatgatccaacaatgggtcaacaacccaatggaggtggatgggagtggagtaactccgatgtgctgaattatcttaactgggatggggatccttcctctactgtcaaccgtggtcactgtgggagtctgacagcaagttcagggtttctgaagtggggagactattactgcgatgggacattaccatttgtctgcaagttcaagcagtagacaagcagcatccagcatttatcatgaagctccccatgacaagggatgaaatacaagaattcacccggcaaggctgtacttgctttacagttgtgcatcagacttattctggttttctgtcctctttcatccatctccttccccttacttcaggcttttcaatatagttcctgctttgcaatcttgcagataaataataaatacaacattttggttttacttttgtgtttt

SEQ ID NO: 44 - protein, 175 aa, Mus musculus
MLPHLVLNSISWMLLSCLLFVFQVQGEDFQKEVPSPRTSCPMGYKAYRSHCYALVMTPKSWFQADLVCQKRPSGHLVSILSGGEASFVSSLVNGRVDNYQDIWIGLHDPTMGQQPNGGGWEWSNSDVLNYLNWDGDPSSTVNRGHCGSLTASSGFLKWGDYYCDGTLPFVCKFKQ

SEQ ID NO: 45 - DNA, 788 bp, Mus musculus
cccggggcgaaatcacctctgagctgtcaaagcattgcagacctctgtatagacagatataccatggtgtctcacaagacccttcatagcatgtcctggatgctactgtgttgcctgatgtccctttcttgggtacaaggggaacaatcccagaaaaaactgtcttctccacgcatcagctgtccccaagaagcccaagcttatggctcctattgctatttactgattctggaaccacagacctgggctaatgcagagatccactgccagaagcatttctcaggacacctggcatttctgctcacttatggggaaattatctttgtgtcctctctggtgaaaaacagtttgaccacattcccatacatctggattggactccatgatctgtcacttgggagtttgcccaatgaaaatggatggaagtggagcagctctgaccctctgaccttctataactgggagatcccaccctccatgtctgcacaccacggttactgcgcagctttgtctcaggcctcaggttatcagaagtggagagattattattgtgacccaacatttccctatgtctgcaaattcaagggttaggccagttctgatttcaactgcctgaaagtatcctgaagatcacatagacaaaggagcgagcatgatggctcaccaagaaagtccttctcacaccccgacaccgaattcctcatctcatctctgctgttcttccataagtgtattctctggggactctggcctaaggattcggagaactataataaaatttagtcaat

SEQ ID NO: 46 - protein, 175 aa, Mus musculus
MVSHKTLHSMSWMLLCCLMSLSWVQGEQSQKKLSSPRISCPQEAQAYGSYCYLLILEPQTWANAEIHCQKHFSGHLAFLLTYGEIIFVSSLVKNSLTTFPYIWIGLHDLSLGSLPNENGWKWSSSDPLTFYNWEIPPSMSAHHGYCAALSQASGYQKWRDYYCDPTFPYVCKFKG

SEQ ID NO: 47 - DNA, 1024 bp, Mus musculus
ccgactcaagctgaaaaaggcagggttctcggagtgttgcttccaatcagattgcaggtctaggagaatccacaaaaaagagaagaaaagaaaagaaaaaaactggaacgggctctgagggccttgaaatcaaagcattgaagtatcatctccatcgaaagaggaagatggcttacaaaggcgtgcggctactcttactgctgagctgggtagctggccccgaagtcctgagcgatatcttgagacccagctgtgccccaggatggttttactataggtcccactgctatggatacttccggaagctaagaaactggtctcatgctgagctggagtgtcagtcatatggaaatggatcccatctggcatctgtcttgaatcaaaaggaagccagtgtcatatcaaagtacataactggctatcagagaaacctgcctgtgtggattggcctgcatgacccacaaaagaagcaattatggcagtggactgatgggtctacaaacctgtacagacgctggaatcccaggacaaagagtgaagccaggcattgcgctgagatgaaccccaaggataaattcttaacttggaacaaaaatggatgtgccaaccgccaacacttcctgtgcaagtataagacatagagcaaaaatcaagcgtctaccagccttgcacaaactcttcccacttccctctcacctggtggctgatctaatcattatcccagagtaaacactgtagcaaacattgaggaggcctccagggcactggctatcaagccctgcttagcatggtgggacagtggcttccggtctcagagtttagcatggtgggacggtgacttccggtctcagagattagcatggtgggacaagggcttctggtctccgtgttcactctacaatcctttctggtactccccttccctctcattgtcttaaacagcaatgcttaacaagctagaaatgtgctttcttgactactgcgtctctgtcaaaccagtaaagttttggagccaagaaacagc

SEQ ID NO: 48 - protein, 157 aa, Mus musculus
MAYKGVRLLLLLSWVAGPEVLSDILRPSCAPGWFYYRSHCYGYFRKLRNWSHAELECQSYGNGSHLASVLNQKEASVISKYITGYQRNLPVWIGLHDPQKKQLWQWTDGSTNLYRRWNPRTKSEARHCAEMNPKDKFLTWNKNGCANRQHFLCKYKT

SEQ ID NO: 49 - DNA, 618 bp, Homo sapiens
atgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtag

SEQ ID NO: 50 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 51 - DNA, 735 bp, Homo sapiens
atgggggcactggggctggagggcaggggtgggaggctccaggggaggggttccctcctgctagctgtggcaggagccacttctctggtgaccttgttgctggcggtgcctatcactgtcctggctgtgctggccttagtgccccaggatcagggaggactggtaacggagacggccgaccccggggcacaggcccagcaaggactggggtttcagaagctgccagaggaggagccagaaacagatctcagccccgggctcccagctgcccacctcataggcgctccgctgaaggggcaggggctaggctgggagacgacgaaggaacaggcgtttctgacgagcgggacgcagttctcggacgccgaggggctggcgctcccgcaggacggcctctattacctctactgtctcgtcggctaccggggccgggcgccccctggcggcggggacccccagggccgctcggtcacgctgcgcagctctctgtaccgggcggggggcgcctacgggccgggcactcccgagctgctgctcgagggcgccgagacggtgactccagtgctggacccggccaggagacaagggtacgggcctctctggtacacgagcgtggggttcggcggcctggtgcagctccggaggggcgagagggtgtacgtcaacatcagtcaccccgatatggtggacttcgcgagagggaagaccttctttggggccgtgatggtggggtga

SEQ ID NO: 52 - protein, 240 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFRKTFFGAVMVG

SEQ ID NO: 53 - DNA, 609 bp, Mus musculus
atgacactgctcggccgtctccacctcttgagggtgcttggcacccctcctgtcttcctcctggggctgctgctggccctgcctctaggggcccagggactctctggtgtccgcttctccgctgccaggacagcccatccactccctcagaagcacttgacccatggcatcctgaaacctgctgctcaccttgttgggtaccccagcaagcagaactcactgctctggagagcaagcacggatcgtgcctttctccgacatggcttctctttgagcaacaactccctcctgatccccaccagtggcctctactttgtctactcccaggtggttttctctggagaaagctgctcccccagggccattcccactcccatctacctggcacacgaggtccagctcttttcctcccaataccccttccatgtgcctctcctcagtgcgcagaagtctgtgtatccgggacttcaaggaccgtgggtgcgctcaatgtaccagggggctgtgttcctgctcagtaagggagaccagctgtccacccacaccgacggcatctcccatctacacttcagccccagcagtgtattctttggagcctttgcactgtag

SEQ ID NO: 54 - protein, 200 aa, Mus musculus
MTLLGRLHLLRVLGTPPVFLLGLLLALPLGAQGLSGVRFSAARTAHPLPQKHLTHGILKPAAHLVGYPSKQNSLLWRASTDRAFLRHGFSLSNNSLLIPTSGFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFAL

SEQ ID NO: 55 - DNA, 609 bp, Mus musculus; identical to SEQ ID NO: 53

SEQ ID NO: 56 - protein, 306 aa, Mus musculus
MGTRGLQGLGGRPQGRGCLLLAVAGATSLVTLLLAVPITVLAVLALVPQDQGRRVEKIIGSGAQAQKRLDDSKPSCILPSPSSLSETPDPRLHPQRSNASRNLASTSQGPVAQSSREASAWMTILSPAADSTPDPGVQQLPKGEPETDLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 57 - DNA, 672 bp, Homo sapiens
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggcagggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaggg

SEQ ID NO: 58 - DNA, 706 bp, Homo sapiens
cgcgcccaggtcaccgacaaagctgcgcactatactctgtgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaat

SEQ ID NO: 59 - DNA, 640 bp, Mus musculus
atgcgcctgccccgggcctcctctccctgcggcctggcctgggggccactcctgctgggactcagcgggcttctggtggcctctcagccccagctggtgcccccttatcgcatagaaaaccagacttgctgggaccaggacaaggaatactacgagcccatgcacgacgtctgctgctcccgctgtcccccaggcgagtttgtctttgcggtatgcagccgcagccaagacacggtttgcaagacttgcccccataattcctataatgaacactggaaccatctctccacctgccagctgtgccgcccctgtgacattgtgctgggctttgaggaggttgccccttgcaccagcgatcggaaagccgagtgccgctgtcagccggggatgtcctgtgtgtatctggacaatgagtgtgtgcactgtgaggaggagcggcttgtactctgccagcctggcacagaagccgaggtcacagatgaaattatggatactgacgtcaactgtgtcccctgtaagccgggacacttccagaacacttcctcccctcgagcccgctgtcaaccccataccagatgtgagatccagggcctggtggaggcagctccaggtacctcctactcggataccatctgtaaaa

SEQ ID NO: 60 - DNA, 718 bp, Mus musculus
ctcgaggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacgacagtactctacgcgtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgaaagcttggccgccatggccc

SEQ ID NO: 61 - protein, 178 aa, Homo sapiens
MKHQHQHQHQHQHQMHQAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 62 - DNA, 24 bp, Artificial Sequence
  source: /note=“Description of Artificial Sequence Synthetic primer”
gtggaagctacttgatgagtaggg

SEQ ID NO: 63 - DNA, 24 bp, Artificial Sequence
  source: /note=“Description of Artificial Sequence Synthetic primer”
agatgcgaaaatggagattaaaag

SEQ ID NO: 64 - DNA, 23 bp, Artificial Sequence
  source: /note=“Description of Artificial Sequence Synthetic primer”
ctacccgtgatattgctgaagag

SEQ ID NO: 65 - DNA, 20 bp, Artificial Sequence
  source: /note=“Description of Artificial Sequence Synthetic primer”
gagcctgaagaagctggaaa

SEQ ID NO: 66 - DNA, 20 bp, Artificial Sequence
  source: /note=“Description of Artificial Sequence Synthetic primer”
caagtgttggcagagatgga

SEQ ID NO: 67 - DNA, 20 bp, Artificial Sequence
  source: /note=“Description of Artificial Sequence Synthetic primer”
tcgccttcttgacgagttct

SEQ ID NO: 68 - DNA, 20 bp, Artificial Sequence
  source: /note=“Description of Artificial Sequence Synthetic primer”
tccgaggagtcagtgctaaa

SEQ ID NO: 69 - DNA, 20 bp, Artificial Sequence
  source: /note=“Description of Artificial Sequence Synthetic primer”
agaacgtcttccagggtgaa

SEQ ID NO: 70 - DNA, 25 bp, Artificial Sequence
  source: /note=“Description of Artificial Sequence Synthetic probe”
tgagcacctgcttcatcaggtagca

SEQ ID NO: 71 - DNA, 19 bp, Artificial Sequence
  source: /note=“Description of Artificial Sequence Synthetic primer”
gctccagaaggccctcaga

SEQ ID NO: 72 - DNA, 19 bp, Artificial Sequence
  source: /note=“Description of Artificial Sequence Synthetic primer”
ctttccctccgcattgaca

SEQ ID NO: 73 - DNA, 21 bp, Artificial Sequence
  source: /note=“Description of Artificial Sequence Synthetic probe”
acctcaaccgttccacgtcac

SEQ ID NO: 74 - DNA, 19 bp, Artificial Sequence
  source: /note=“Description of Artificial Sequence Synthetic primer”
gcatcctcatggagcacat

SEQ ID NO: 75 - DNA, 18 bp, Artificial Sequence
  source: /note=“Description of Artificial Sequence Synthetic primer”
ctggtcagccaggagctt

SEQ ID NO: 76 - DNA, 21 bp, Artificial Sequence
  source: /note=“Description of Artificial Sequence Synthetic probe”
cttgcgggccttgtctgcctt

SEQ ID NO: 77 - DNA, 20 bp, Artificial Sequence
  source: /note=“Description of Artificial Sequence Synthetic primer”
agcctggattgacaggaatc

SEQ ID NO: 78 - DNA, 21 bp, Artificial Sequence
  source: /note=“Description of Artificial Sequence Synthetic primer”
gataatcagacgaggcgtttc

SEQ ID NO: 79 - DNA, 25 bp, Artificial Sequence
  source: /note=“Description of Artificial Sequence Synthetic probe”
tctggaaactcctgcagcctgacac

SEQ ID NO: 80 - DNA, 22 bp, Artificial Sequence
  source: /note=“Description of Artificial Sequence Synthetic primer”
tttgggagaactaggcattctt

SEQ ID NO: 81 - DNA, 21 bp, Artificial Sequence
  source: /note=“Description of Artificial Sequence Synthetic primer”
tcttggacaggagtgttctca

SEQ ID NO: 82 - DNA, 31 bp, Artificial Sequence
  source: /note=“Description of Artificial Sequence Synthetic probe”
cagcctctccactttcatctatagcatctcc

SEQ ID NO: 83 - DNA, 19 bp, Artificial Sequence
  source: /note=“Description of Artificial Sequence Synthetic primer”
gctctccatgccatttcaa

SEQ ID NO: 84 - DNA, 18 bp, Artificial Sequence
  source: /note=“Description of Artificial Sequence Synthetic primer”
tggccaagggtctgaagt

SEQ ID NO: 85 - DNA, 25 bp, Artificial Sequence
  source: /note=“Description of Artificial Sequence Synthetic probe”
tgtacatccctgctgtcctcaaggc

SEQ ID NO: 86 - DNA, 24 bp, Artificial Sequence
  source: /note=“Description of Artificial Sequence Synthetic primer”
tccaatgctctcctaacagataag

SEQ ID NO: 87 - DNA, 22 bp, Artificial Sequence
  source: /note=“Description of Artificial Sequence Synthetic primer”
caagatgaattggatggtcttg

SEQ ID NO: 88 - DNA, 27 bp, Artificial Sequence
  source: /note=“Description of Artificial Sequence Synthetic probe”
tccttagccactccttctgtgactcca

SEQ ID NO: 89 - DNA, 26 bp, Artificial Sequence
  source: /note=“Description of Artificial Sequence Synthetic primer”
tgtcctcagtttgtgcagaatataaa

SEQ ID NO: 90 - DNA, 20 bp, Artificial Sequence
  source: /note=“Description of Artificial Sequence Synthetic primer”
tcaccatcgcaaggaactcc

SEQ ID NO: 91 - DNA, 36 bp, Artificial Sequence
  source: /note=“Description of Artificial Sequence Synthetic probe”
cgaaaacttgttcagagaattggacatcaatagtga

SEQ ID NO: 92 - DNA, 19 bp, Artificial Sequence
  source: /note=“Description of Artificial Sequence Synthetic primer”
ggtggaagcacagttggca

SEQ ID NO: 93 - DNA, 21 bp, Artificial Sequence
  source: /note=“Description of Artificial Sequence Synthetic primer”
gtgtccaggtcctccatgatg

SEQ ID NO: 94 - DNA, 36 bp, Artificial Sequence
  source: /note=“Description of Artificial Sequence Synthetic probe”
tgaagaaagagaagagaaatgaagccctcataaatg

SEQ ID NO: 95 - DNA, 20 bp, Artificial Sequence
  source: /note=“Description of Artificial Sequence Synthetic primer”
atggctcctattgctatgcc

SEQ ID NO: 96 - DNA, 19 bp, Artificial Sequence
  source: /note=“Description of Artificial Sequence Synthetic primer”
gatgtcctgagggcctctt

SEQ ID NO: 97 - DNA, 25 bp, Artificial Sequence
  source: /note=“Description of Artificial Sequence Synthetic probe”
tggcaggccatatctgcatcatacc

SEQ ID NO: 98 - DNA, 20 bp, Artificial Sequence
  source: /note=“Description of Artificial Sequence Synthetic primer”
atggctcctactgctatgcc

SEQ ID NO: 99 - DNA, 19 bp, Artificial Sequence
  source: /note=“Description of Artificial Sequence Synthetic primer”
gtgtcctccaggcctcttt

SEQ ID NO: 100 - DNA, 21 bp, Artificial Sequence
  source: /note=“Description of Artificial Sequence Synthetic probe”
tgatgcagaactggcctgcca

SEQ ID NO: 101 - DNA, 22 bp, Artificial Sequence
  source: /note=“Description of Artificial Sequence Synthetic primer”
acatctaccgaagtccaatgca

SEQ ID NO: 102 - DNA, 24 bp, Artificial Sequence
  source: /note=“Description of Artificial Sequence Synthetic primer”
ggaattgtaatagcgatcctgagc

SEQ ID NO: 103 - DNA, 21 bp, Artificial Sequence
  source: /note=“Description of Artificial Sequence Synthetic probe”
tgcacgcagacattcccgcct

SEQ ID NO: 104 - DNA, 18 bp, Artificial Sequence
  source: /note=“Description of Artificial Sequence Synthetic primer”
ggtggctcagggaaatgt

SEQ ID NO: 105 - DNA, 20 bp, Artificial Sequence
  source: /note=“Description of Artificial Sequence Synthetic primer”
gacagagcaggcaggtacag

SEQ ID NO: 106 - DNA, 27 bp, Artificial Sequence
  source: /note=“Description of Artificial Sequence Synthetic probe”
cagatgcacagtactccagacagcagc

SEQ ID NO: 107 - DNA, 20 bp, Artificial Sequence
  source: /note=“Description of Artificial Sequence Synthetic primer”
caggtgcttccagtccgtct

SEQ ID NO: 108 - DNA, 21 bp, Artificial Sequence
  source: /note=“Description of Artificial Sequence Synthetic primer”
ctctcctggaatccccaaagt

SEQ ID NO: 109 - DNA, 24 bp, Artificial Sequence
  source: /note=“Description of Artificial Sequence Synthetic probe”
cagcacagatgccaacggcctcat

SEQ ID NO: 110 - DNA, 18 bp, Artificial Sequence
  source: /note=“Description of Artificial Sequence Synthetic primer”
ctggccgcttcgggacgc

SEQ ID NO: 111 - DNA, 24 bp, Artificial Sequence
  source: /note=“Description of Artificial Sequence Synthetic primer”
aaccacagaagacacaaggaactg

SEQ ID NO: 112 - DNA, 20 bp, Artificial Sequence
  source: /note=“Description of Artificial Sequence Synthetic probe”
tctgctgctggccgcttcgg

SEQ ID NO: 113 - DNA, 19 bp, Artificial Sequence
  source: /note=“Description of Artificial Sequence Synthetic primer”
gctggactcccttgtgtgt

SEQ ID NO: 114 - DNA, 19 bp, Artificial Sequence
  source: /note=“Description of Artificial Sequence Synthetic primer”
cacatggcctcagtctcaa

SEQ ID NO: 115 - DNA, 20 bp, Artificial Sequence
  source: /note=“Description of Artificial Sequence Synthetic probe”
cgcgggaccctcatcctttg

SEQ ID NO: 116 - DNA, 21 bp, Artificial Sequence
  source: /note=“Description of Artificial Sequence Synthetic primer”
tccacagcacctgaaggagtt

SEQ ID NO: 117 - DNA, 21 bp, Artificial Sequence
  source: /note=“Description of Artificial Sequence Synthetic primer”
ggagggaaggagaacagcaga

SEQ ID NO: 118 - DNA, 20 bp, Artificial Sequence
  source: /note=“Description of Artificial Sequence Synthetic probe”
tgggccacccccatcacagc